SEQUENCE LISTING (192 sequences)

SEQ ID NO: 1 - protein, 354 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 355 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 486 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 528 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 353 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDFWVLVVVGGVLACYSLLVTVAFIIFWVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 354 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 485 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 527 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 529 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 660 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 528 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 659 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 485 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 487 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 659 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 661 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 484 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDFWVLVVVGGVLACYSLLVTVAFIIFWVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 486 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 658 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 660 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 431 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 335 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNLCARPRRSPAQEDGKVYINMPGRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 363 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNYFLGRLVPRGRGAAEAATRKQRITETESPYQELQGQRSDVYSDLNTQRPYYKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 353 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 291 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 26 - protein, 294 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELYFLGRLVPRGRGAAEAATRKQRITETESPYQELQGQRSDVYSDLNTQRPYYK

SEQ ID NO: 27 - protein, 293 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNALYLLRRDQRLPPDAHKPPGGGSFRTPIQEEQADAHSTLAKIYFLGRLVPRGRGAAEAATRKQRITETESPYQELQGQRSDVYSDLNTQRPYYK

SEQ ID NO: 28 - protein, 281 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRLKIQVRKAAITSYEKSDGVYTGLSTRNQETYETLKHEKPPQ

SEQ ID NO: 29 - protein, 311 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 351 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 373 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 432 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 336 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNLCARPRRSPAQEDGKVYINMPGRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 364 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNYFLGRLVPRGRGAAEAATRKQRITETESPYQELQGQRSDVYSDLNTQRPYYKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 354 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 292 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 37 - protein, 295 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELYFLGRLVPRGRGAAEAATRKQRITETESPYQELQGQRSDVYSDLNTQRPYYK

SEQ ID NO: 38 - protein, 294 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNALYLLRRDQRLPPDAHKPPGGGSFRTPIQEEQADAHSTLAKIYFLGRLVPRGRGAAEAATRKQRITETESPYQELQGQRSDVYSDLNTQRPYYK

SEQ ID NO: 39 - protein, 282 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRLKIQVRKAAITSYEKSDGVYTGLSTRNQETYETLKHEKPPQ

SEQ ID NO: 40 - protein, 312 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 352 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 374 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 563 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 467 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNLCARPRRSPAQEDGKVYINMPGRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 495 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNYFLGRLVPRGRGAAEAATRKQRITETESPYQELQGQRSDVYSDLNTQRPYYKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 485 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 423 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 48 - protein, 426 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELYFLGRLVPRGRGAAEAATRKQRITETESPYQELQGQRSDVYSDLNTQRPYYK

SEQ ID NO: 49 - protein, 425 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNALYLLRRDQRLPPDAHKPPGGGSFRTPIQEEQADAHSTLAKIYFLGRLVPRGRGAAEAATRKQRITETESPYQELQGQRSDVYSDLNTQRPYYK

SEQ ID NO: 50 - protein, 413 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRLKIQVRKAAITSYEKSDGVYTGLSTRNQETYETLKHEKPPQ

SEQ ID NO: 51 - protein, 443 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 483 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 505 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 562 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 466 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNLCARPRRSPAQEDGKVYINMPGRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 494 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNYFLGRLVPRGRGAAEAATRKQRITETESPYQELQGQRSDVYSDLNTQRPYYKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 484 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 422 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 59 - protein, 425 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELYFLGRLVPRGRGAAEAATRKQRITETESPYQELQGQRSDVYSDLNTQRPYYK

SEQ ID NO: 60 - protein, 424 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNALYLLRRDQRLPPDAHKPPGGGSFRTPIQEEQADAHSTLAKIYFLGRLVPRGRGAAEAATRKQRITETESPYQELQGQRSDVYSDLNTQRPYYK

SEQ ID NO: 61 - protein, 412 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRLKIQVRKAAITSYEKSDGVYTGLSTRNQETYETLKHEKPPQ

SEQ ID NO: 62 - protein, 442 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 482 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 504 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 564 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 468 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNLCARPRRSPAQEDGKVYINMPGRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 496 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNYFLGRLVPRGRGAAEAATRKQRITETESPYQELQGQRSDVYSDLNTQRPYYKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 486 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 424 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 70 - protein, 427 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELYFLGRLVPRGRGAAEAATRKQRITETESPYQELQGQRSDVYSDLNTQRPYYK

SEQ ID NO: 71 - protein, 426 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNALYLLRRDQRLPPDAHKPPGGGSFRTPIQEEQADAHSTLAKIYFLGRLVPRGRGAAEAATRKQRITETESPYQELQGQRSDVYSDLNTQRPYYK

SEQ ID NO: 72 - protein, 414 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRLKIQVRKAAITSYEKSDGVYTGLSTRNQETYETLKHEKPPQ

SEQ ID NO: 73 - protein, 444 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 484 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 506 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSFVPVFLPAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 485 aa, synthetic construct; identical to SEQ ID NO: 13

SEQ ID NO: 77 - protein, 659 aa, synthetic construct; identical to SEQ ID NO: 15

SEQ ID NO: 78 - protein, 484 aa, synthetic construct; identical to SEQ ID NO: 17

SEQ ID NO: 79 - protein, 658 aa, synthetic construct; identical to SEQ ID NO: 19

SEQ ID NO: 80 - protein, 562 aa, synthetic construct; identical to SEQ ID NO: 54

SEQ ID NO: 81 - protein, 466 aa, synthetic construct; identical to SEQ ID NO: 55

SEQ ID NO: 82 - protein, 494 aa, synthetic construct; identical to SEQ ID NO: 56

SEQ ID NO: 83 - protein, 484 aa, synthetic construct; identical to SEQ ID NO: 57

SEQ ID NO: 84 - protein, 422 aa, synthetic construct; identical to SEQ ID NO: 58

SEQ ID NO: 85 - protein, 425 aa, synthetic construct; identical to SEQ ID NO: 59

SEQ ID NO: 86 - protein, 424 aa, synthetic construct; identical to SEQ ID NO: 60

SEQ ID NO: 87 - protein, 412 aa, synthetic construct; identical to SEQ ID NO: 61

SEQ ID NO: 88 - protein, 442 aa, synthetic construct; identical to SEQ ID NO: 62

SEQ ID NO: 89 - protein, 482 aa, synthetic construct; identical to SEQ ID NO: 63

SEQ ID NO: 90 - protein, 504 aa, synthetic construct; identical to SEQ ID NO: 64

SEQ ID NO: 91 - protein, 8 aa, synthetic construct
GIDLSLYR

SEQ ID NO: 92 - protein, 7 aa, synthetic construct
ITDDGTS

SEQ ID NO: 93 - protein, 11 aa, synthetic construct
NAETPLSPVNY

SEQ ID NO: 94 - protein, 8 aa, synthetic construct
GSIFGIRT

SEQ ID NO: 95 - protein, 7 aa, synthetic construct
ITMDGRV

SEQ ID NO: 96 - protein, 11 aa, synthetic construct
RYSGLTSREDY

SEQ ID NO: 97 - protein, 116 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVS

SEQ ID NO: 98 - protein, 117 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 99 - protein, 248 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 100 - protein, 247 aa, synthetic construct
QVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVSGGGGSGGGGSGGGGSQVQLVQSGGGLVHPGGSLRLSCAASGIDLSLYRMRWYRQAPGKERDLVALITDDGTSYYEDSVKGRFTITRDNPSNKVFLQMNSLKPEDTAVYYCNAETPLSPVNYWGQGTQVTVS

SEQ ID NO: 101 - protein, 249 aa, synthetic construct
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSGGGGSGGGGSGGGGSQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 102 - protein, 247 aa, synthetic construct; identical to SEQ ID NO: 100

SEQ ID NO: 103 - protein, 116 aa, synthetic construct
QVQLVESGGGLVQPGGSLRLSCAASGIDLSLYRLGWYRQAPGQGLDLVALITDDGTSYYADSVKGRFTISRDNSKNTLYLQMNSLRAEDTAVYYCNAETPLSPVNYWGQGTLVTVS

SEQ ID NO: 104 - protein, 80 aa, synthetic construct
QVQLVESGGGLVQPGGSLRLSCAASGSIFGIRTLGWYRQAPGQGLELVARITMDGRVYYADSVKGRFTISRDNSKNTLYL

SEQ ID NO: 105 - protein, 45 aa, Homo sapiens
TTTPAPRPPTPAPTIASQPLSLRPEACRPAAGGAVHTRGLDFACD

SEQ ID NO: 106 - protein, 55 aa, Homo sapiens
FVPVFLPAKPTTTPAPRPPTPAPTIASQPLSLRPEACRPAAGGAVHTRGLDFACD

SEQ ID NO: 107 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 232 aa, Homo sapiens; identical to SEQ ID NO: 107

SEQ ID NO: 109 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 16 aa, Homo sapiens
GLAVSTISSFFPPGYQ

SEQ ID NO: 111 - protein, 39 aa, Homo sapiens
IEVMYPPPYLDNEKSNGTIIHVKGKHLCPSPLFPGPSKP

SEQ ID NO: 112 - protein, 19 aa, Homo sapiens
AEPKSPDKTHTCPPCPKDP

SEQ ID NO: 113 - protein, 12 aa, Homo sapiens
ESKYGPPCPSCP

SEQ ID NO: 114 - protein, 119 aa, Homo sapiens
ESKYGPPCPSCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 229 aa, Homo sapiens
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 229 aa, Homo sapiens
ESKYGPPCPSCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 21 aa, Homo sapiens
IYIWAPLAGTCGVLLLSLVIT

SEQ ID NO: 119 - protein, 28 aa, Homo sapiens
IYIWAPLAGTCGVLLLSLVITLYCNHRN

SEQ ID NO: 120 - protein, 27 aa, Homo sapiens
FWVLVVVGGVLACYSLLVTVAFIIFWV

SEQ ID NO: 121 - protein, 23 aa, Homo sapiens
VVRVLAIALAIRFTLNTLMWLAI

SEQ ID NO: 122 - protein, 30 aa, Homo sapiens
PFFFCCFIAVAMGIRFIIMVAIWSAVFLNS

SEQ ID NO: 123 - protein, 21 aa, Homo sapiens
VSFCLVMVLLFAVDTGLYFSV

SEQ ID NO: 124 - protein, 21 aa, Homo sapiens
LVPVFCGLLVAKSLVLSALLV

SEQ ID NO: 125 - protein, 21 aa, Homo sapiens
MGLAFLVLVALVWFLVEDWLS

SEQ ID NO: 126 - protein, 20 aa, Homo sapiens
ILVIFSGMFLVFTLAGALFL

SEQ ID NO: 127 - protein, 21 aa, Homo sapiens
ILVIFSGMFLVFTLAGALFLH

SEQ ID NO: 128 - protein, 21 aa, Homo sapiens
GVLAGIVMGDLVLTVLIALAV

SEQ ID NO: 129 - protein, 21 aa, Homo sapiens
LLAGLVAADAVASLLIVGAVF

SEQ ID NO: 130 - protein, 4 aa, synthetic construct
GGGS

SEQ ID NO: 131 - protein, 5 aa, synthetic construct
GGGGS

SEQ ID NO: 132 - protein, 20 aa, synthetic construct
GGGGSGGGGSGGGGSGGGGS

SEQ ID NO: 133 - protein, 15 aa, synthetic construct
GGGGSGGGGSGGGGS

SEQ ID NO: 134 - protein, 41 aa, Homo sapiens
RSKRSRGGHSDYMNMTPRRPGPTRKHYQPYAPPRDFAAYRS

SEQ ID NO: 135 - protein, 41 aa, Homo sapiens
RSKRSRLLHSDYMNMTPRRPGPTRKHYQPYAPPRDFAAYRS

SEQ ID NO: 136 - protein, 42 aa, Homo sapiens
KRGRKKLLYIFKQPFMRPVQTTQEEDGCSCRFPEEEEGGCEL

SEQ ID NO: 137 - protein, 43 aa, Homo sapiens
RKRGRKKLLYIFKQPFMRPVQTTQEEDGCSCRFPEEEEGGCEL

SEQ ID NO: 138 - protein, 24 aa, Homo sapiens
LCARPRRSPAQEDGKVYINMPGRG

SEQ ID NO: 139 - protein, 23 aa, Homo sapiens
CARPRRSPAQEDGKVYINMPGRG

SEQ ID NO: 140 - protein, 52 aa, Homo sapiens
YFLGRLVPRGRGAAEAATRKQRITETESPYQELQGQRSDVYSDLNTQRPYYK

SEQ ID NO: 141 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 142 - protein, 42 aa, Homo sapiens
ALYLLRRDQRLPPDAHKPPGGGSFRTPIQEEQADAHSTLAKI

SEQ ID NO: 143 - protein, 49 aa, Homo sapiens
HQRRKYRSNKGESPVEPAEPCHYSCPREEEGSTIPIQEDYRKPEPACSP

SEQ ID NO: 144 - protein, 48 aa, Homo sapiens
QRRKYRSNKGESPVEPAEPCHYSCPREEEGSTIPIQEDYRKPEPACSP

SEQ ID NO: 145 - protein, 22 aa, Homo sapiens
ERVQPLEENVGNAARPRFERNK

SEQ ID NO: 146 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 42 aa, Homo sapiens
RLKIQVRKAAITSYEKSDGVYTGLSTRNQETYETLKHEKPPQ

SEQ ID NO: 149 - protein, 77 aa, Homo sapiens
CRKKRISANSTDPVKAAQFEPPGRQMIAIRKRQLEETNNDYETADGGYMTLNPRAPTDDDKNIYLTLPPNDHVNSNN

SEQ ID NO: 150 - protein, 21 aa, Homo sapiens
MALPVTALLLPLALLLHAARP

SEQ ID NO: 151 - protein, 19 aa, Homo sapiens
MARPHPWWLCVLGTLVGLS

SEQ ID NO: 152 - protein, 21 aa, Homo sapiens
MEFGLSWLFLVAILKGVQCSR

SEQ ID NO: 153 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 154 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 155 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 156 - protein, 237 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 157 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 158 - protein, 65 aa, synthetic construct
TTCPPPVSIEHADIRVKNYSVNSRERYVCNSGFKRKAGTSTLIECVINKNTNVAHWTTPSLKCIR

SEQ ID NO: 159 - protein, 201 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 160 - protein, 183 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 161 - protein, 402 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGGSGGGGSGGGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 162 - protein, 384 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGGSGGGGSGGGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 163 - protein, 224 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSGSGSGSGSGSGSGSGSGSGSGSGSGSVAISTSTVLLCGLSAVSLLACYLKSRQTPPLASVEMEAMEALPVTWGTSSRDEDLENCSHHL

SEQ ID NO: 164 - protein, 224 aa, synthetic construct; identical to SEQ ID NO: 163

SEQ ID NO: 165 - protein, 5 aa, synthetic construct
GGGGS

SEQ ID NO: 166 - protein, 15 aa, synthetic construct
GGGGSGGGGSGGGGS

SEQ ID NO: 167 - protein, 25 aa, synthetic construct
GGSGGSGGYPYDVPDYAGGGSGGGS

SEQ ID NO: 168 - protein, 24 aa, synthetic construct
GGSGGSGGGGGSGGGSGGGSGGGS

SEQ ID NO: 169 - protein, 26 aa, synthetic construct
GGSGGSGGGPEDEPGSGSGGGSGGGS

SEQ ID NO: 170 - protein, 42 aa, synthetic construct
GGSGGSGGGGGSGGGSGGGSGGGSGSGSGSGSEDGSGSGSGS

SEQ ID NO: 171 - protein, 28 aa, synthetic construct
GSGSGSGSGSEDEDEDEDGSGSGSGSGS

SEQ ID NO: 172 - protein, 20 aa, synthetic construct
GGGGSGGGGSGGGGSGGGGS

SEQ ID NO: 173 - protein, 18 aa, synthetic construct
GSGSGSGSEDGSGSGSGS

SEQ ID NO: 174 - protein, 18 aa, synthetic construct
GSGSGSGSGSGSGSGSGS

SEQ ID NO: 175 - protein, 15 aa, synthetic construct
GCGGSGGGGSGGGGS

SEQ ID NO: 176 - protein, 24 aa, synthetic construct
GGSGGSGGGGGSGGGSGGGSGGGS

SEQ ID NO: 177 - protein, 26 aa, synthetic construct
SGRGGGGSGGGGSGGGGSGGGGSSPA

SEQ ID NO: 178 - protein, 24 aa, synthetic construct
GGGGSGGGGSGGGGSGGGGSGGGG

SEQ ID NO: 179 - protein, 58 aa, synthetic construct
SGRGASSGSSGSGSQKKPRYEIRWKVVVISAILALVVLTVISLIILIMLWGSGMQSPA

SEQ ID NO: 180 - protein, 21 aa, synthetic construct
GSGEGRGSLLTCGDVEENPGP

SEQ ID NO: 181 - protein, 22 aa, synthetic construct
GSGATNFSLLKQAGDVEENPGP

SEQ ID NO: 182 - protein, 23 aa, synthetic construct
GSGQCTNYALLKLAGDVESNPGP

SEQ ID NO: 183 - protein, 25 aa, synthetic construct
GSGVKQTLNFDLLKLAGDVESNPGP

SEQ ID NO: 184 - protein, 165 aa, synthetic construct
GRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 185 - protein, 144 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 186 - protein, 199 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 187 - protein, 177 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 188 - protein, 205 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVCYRVNRQQKLSS

SEQ ID NO: 189 - protein, 183 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWVLVVVGGVLACYSLLVTVAFIIFWVCYRVNRQQKLSS

SEQ ID NO: 190 - protein, 93 aa, synthetic construct
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 191 - protein, 21 aa, Homo sapiens
AAVIAGPVCFVCISLMLMVYI

SEQ ID NO: 192 - protein, 21 aa, Homo sapiens
VTGISLLPPLGVAISVIIIFY

(19) United States
(12) Patent Application Publication
(10) Pub. No.: US 2024/0156867 A1
(43) Pub. Date: May 16, 2024

(54) ENGINEERED IMMUNE CELL THERAPEUTICS AND METHODS OF USE

(71) Applicant: Catamaran Bio, Inc., Cambridge, MA (US)

(72) Inventors: Jared Allan Sewell, Somerville, MA (US); Celeste Jeanne Richardson, Brookline, MA (US)

(21) Appl. No.: 18/506,708

(22) Filed: Nov. 10, 2023

Related U.S. Application Data
(60) Provisional application No. 63/424,700, filed on Nov. 11, 2022.

(51) Int. Cl. A61K 35/17; A61K 39/00; A61K 45/06; C07K 14/495; C07K 14/54; C12N 5/0783

(52) U.S. Cl. CPC A61K 35/17; A61K 39/4611; A61K 39/4613; A61K 39/4631; A61K 39/464434; A61K 39/46444; A61K 45/06; C07K 14/495; C07K 14/5443; C12N 5/0636; C12N 5/0646; A61K 2239/21

(57) ABSTRACT

This disclosure describes chimeric antigen receptors (CARs) engineered immune cells including the CARs, pharmaceutical compositions that include engineered immune cells that include the CARs, and methods involving such engineered immune cells.

### CROSS-REFERENCE TO RELATED APPLICATIONS

This application claims priority to U.S. Provisional Patent Application Ser. No. 63/424,700, filed Nov. 11, 2022; the entire contents of which are herein incorporated by reference.

### SEQUENCE LISTING

This application contains a Sequence Listing that has been submitted electronically as an XML file named 52526-0023001_SL_ST26.xml. The XML file, created on Nov. 10, 2023, is 214,532 bytes in size. The material in the XML file is hereby incorporated by reference in its entirety.

### FIELD

The present invention relates generally to the fields of molecular biology, immunology, oncology, and medicine. More particularly, it concerns immune cells expressing chimeric antigen receptors that bind to a target protein.

### BACKGROUND

In recent years, adoptive cellular therapy using autologous T cells expressing chimeric antigen receptors (CARs) has proven to be a very powerful approach for the treatment of cancer, including B cell leukemia and lymphoma. Natural killer (NK) cells mediate effective cytotoxicity against tumor cells and unlike T cells, lack the potential to cause graft versus host disease (GVHD) in the allogeneic setting. Thus, NK cells expressing CARs are a promising candidate for off-the-shelf cellular therapies. In addition, NK cells can also be obtained from various sources, including peripheral blood, cord blood, and the differentiation of inducible pluripotent stem cells (iPSCs) or CD34⁺ hematopoietic stem cells (HSCs). The potent activity of CAR-expressing NK cells has been demonstrated in the preclinical setting with different tumor models, and clinical trials of allogeneic CAR-transduced NK cells are currently underway.

### SUMMARY

Provided herein are chimeric antigen receptor including a sequence that is at least 85% identical to any one of SEQ ID NOs: 1-90.

In some embodiments, the chimeric antigen receptor includes a sequence that is at least 90% identical to any one of SEQ ID NOs: 1-90. In some embodiments, the chimeric antigen receptor includes a sequence that is at least 95% identical to any one of SEQ ID NOs: 1-90. In some embodiments, the chimeric antigen receptor includes a sequence of any one of SEQ ID NOs: 1-90.

Also provided herein are nucleic acids encoding any of the chimeric antigen receptor described herein.

Also provided herein are vectors including any of the nucleic acid described herein.

Also provided herein are engineered immune cells including any of the chimeric antigen receptors described herein.

In some embodiments of any of the engineered immune cells described herein, the engineered immune cell is an engineered NK cell, T cell, or natural killer T (NKT) cell. In some embodiments of any of the engineered immune cells described herein, the engineered immune cell includes one or more exogenous polypeptides.

In some embodiments of any of the engineered immune cells described herein, the one or more exogenous polypeptides is selected from the group consisting of interleukin-15 or a functional fragment thereof, interleukin-15 receptor alpha or a functional fragment thereof, or a transmembrane protein including IL-15 or a functional fragment thereof. In some embodiments of any of the engineered immune cells described herein, the one or more exogenous polypeptides includes the transmembrane protein. In some embodiments of any of the engineered immune cells described herein, the transmembrane protein includes a sushi domain of IL-15 receptor alpha.

In some embodiments of any of the engineered immune cells described herein, the one or more exogenous polypeptides includes interleukin-15 or a functional fragment thereof.

In some embodiments of any of the engineered immune cells described herein, the one or more exogenous polypeptides includes interleukin-15 receptor alpha or a functional fragment thereof. In some embodiments of any of the engineered immune cells described herein, the engineered immune cell includes a first exogenous polypeptide including interleukin-15 or a functional fragment thereof, and a second exogenous polypeptide including interleukin-15 receptor alpha or a functional fragment thereof.

In some embodiments of any of the engineered immune cells described herein, the one or more exogenous polypeptides includes a transforming growth factor (TGF)-β dominant negative receptor. In some embodiments of any of the engineered immune cells described herein, the TGF-β dominant negative receptor includes the extracellular domain of TGF-β type II receptor. In some embodiments of any of the engineered immune cells described herein, the TGF-β dominant negative receptor includes the extracellular domain of TGF-β type I receptor. In some embodiments of any of the engineered immune cells described herein, the TGF-β dominant negative receptor includes the transmembrane domain of TGF-β type I receptor, TGF-β type II receptor, CD28, or CD8α.

Also provided herein are pharmaceutical compositions including any of the engineered immune cells described herein and a pharmaceutically acceptable carrier.

Also provided herein are kits including any of the engineered immune cells and/or pharmaceutical compositions described herein.

Also provided herein are methods of treating a subject having a mesothelin-associated cancer, where the method includes administering to the subject any of the engineered immune cells described herein or any of the pharmaceutical compositions described herein. In some embodiments of any of the methods described herein, the administering is intravenous administration. In some embodiments of any of the methods described herein, the mesothelin-associated cancer is selected from the group consisting of: mesothelioma, ovarian cancer, pancreatic cancer, brain cancer, lung cancer, bladder cancer, breast cancer, cervical cancer, colorectal cancer, head and neck cancer, liver cancer, kidney cancer, lymphoma, leukemia, skin cancer, neuroblastoma, ovarian cancer, thyroid cancer, sarcoma, gastric cancer, pleural cancer, glioblastoma, esophageal cancer, gastric cancer, urothelial cancer, ureter cancer, endometrial cancer, penile cancer, stomach cancer, squamous cell carcinoma, cholangiocarcinoma, and any combination thereof. In some embodiments of any of the methods described herein, the method further comprises administering an additional therapeutic agent to the subject. In some embodiments of any of the methods described herein, then the additional therapeutic agent is a TNF-α converting enzyme (TACE) inhibitor (e.g., any one of ilomastat, batimastat, marimastat, KB-R7785, prinomastat, tanomastat, Ro 32-7315, cipemastat, CGS27023A, TMI-1, and DPC-333).

Also provided herein are methods of making any of the engineered immune cells described herein, the method including: introducing into an immune cell any of the nucleic acids described herein or any of the vectors described herein.

In some embodiments of any of the methods described herein, the immune cell is an NK cell, T cell, or natural killer T (NKT) cell (e.g., an invariant NKT (iNKT) cell).

In some embodiments of any of the methods described herein, the method includes introducing into the immune cell nucleic acid encoding one or more exogenous polypeptides. In some embodiments of any of the methods described herein, the one or more exogenous polypeptides is selected from the group consisting of interleukin-15 or a functional fragment thereof, interleukin-15 receptor alpha or a functional fragment thereof, or a transmembrane protein comprising IL-15 or a functional fragment thereof. In some embodiments of any of the methods described herein, the one or more exogenous polypeptides comprises the transmembrane protein. In some embodiments of any of the methods described herein, the transmembrane protein further comprises a sushi domain of IL-15 receptor alpha.

In some embodiments of any of the methods described herein, the one or more exogenous polypeptides comprises interleukin-15 or a functional fragment thereof. In some embodiments of any of the methods described herein, the one or more exogenous polypeptides comprises interleukin-15 receptor alpha or a functional fragment thereof. In some embodiments of any of the methods described herein, the engineered immune cell comprises a first exogenous polypeptide comprising interleukin-15 or a functional fragment thereof, and a second exogenous polypeptide comprising interleukin-15 receptor alpha or a functional fragment thereof.

In some embodiments of any of the methods described herein, the one or more exogenous polypeptides comprises a transforming growth factor (TGF)-β dominant negative receptor. In some embodiments of any of the methods described herein, the TGF-β dominant negative receptor comprises the extracellular domain of TGF-β type II receptor. In some embodiments of any of the methods described herein, the TGF-β dominant negative receptor comprises the extracellular domain of TGF-β type I receptor. In some embodiments of any of the methods described herein, the TGF-β dominant negative receptor further comprises the transmembrane domain of TGF-β type I receptor, TGF-β type II receptor, CD28, or CD8α.

### DETAILED DESCRIPTION

The present disclosure describes various chimeric antigen receptors (CARs) that target mesothelin, engineered immune cells (e.g., T cells and NK cells) including (e.g., expressing) these CARs, and methods of treating mesothelin-associated cancers by administering these immune cells (or pharmaceutical compositions including the immune cells).

### Definitions

As used herein the specification, “a” or “an” refers to one or to more than one (i.e., to at least one) of the grammatical object of the article.

The term “about” when referring to a measurable value (e.g., amount or duration) is meant to encompass variations of in some instances ±20%, or in some instances ±10%, or in some instances ±5%, or in some instances ±1%, or in some instances ±0.5%, or in some instances ±0.1% from the specified value.

The term “exogenous,” when used in relation to a polypeptide or nucleic acid in a cell or organism refers to a polypeptide or nucleic acid that has been introduced into the cell or organism by artificial or natural mean.

As used herein, the term “mesothelin” refers to the 40 kDa form of mesothelin protein which results from the processing of a mesothelin precursor protein in a mammalian cell (e.g., a human cell). The human mesothelin gene encodes a 71 kDa precursor protein that is proteolytically cleaved by furin into two products, a secreted 31 kDa N-terminal fragment (referred to as megakaryocyte potentiating factor (MPF)), and a 40 kDa protein component (see, e.g., Einama et al. (2016) World J. Gastrointest. Pathophysiol. 7(2): 218-22). The 40 kDa mesothelin component generally remains attached to cell membranes via a glycosylphosphatidylinisotol (GPI) linkage but can also be proteolytically cleaved and shed. Generally, the 40 kDa mesothelin protein component is a cell surface glycoprotein that normally has restricted expression in the mesothelia (e.g., peritoneum, pericardium, and pleura). The term “mesothelin” also includes variants of mesothelin, e.g., splice variants or allelic variants.

The term “mesothelin-associated cancer” refers to a cancer associated with the overexpression (e.g., as compared to a non-cancer cell of the same type) of mesothelin. Examples of cancers that overexpress mesothelin include mesothelioma, ovarian cancer, pancreatic cancer, brain cancer, lung cancer, bladder cancer, breast cancer, cervical cancer, colorectal cancer, head and neck cancer, liver cancer, kidney cancer, lymphoma, leukemia, skin cancer, neuroblastoma, ovarian cancer, thyroid cancer, sarcoma, gastric cancer, pleural cancer, glioblastoma, esophageal cancer, gastric cancer, urothelial cancer, ureter cancer, endometrial cancer, penile cancer, stomach cancer, squamous cell carcinoma, cholangiocarcinoma, and any combination thereof. In some embodiments, a mesothelin-associated cancer is a combination of any of the cancers described herein.

The term “antibody” as used herein refers to a protein, or polypeptide sequence derived from an immunoglobulin molecule, which specifically binds to an antigen. Antibodies can be monoclonal or polyclonal, multiple or single chain, and/or intact immunoglobulins, and may be derived from recombinant or natural sources. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

The term “antibody fragment” refers to at least one portion of an antibody that retains the ability to specifically interact with (e.g., by binding, steric hinderance, stabilizing, destabilizing, and/or spatial hinderance) an epitope of an antigen. Examples of antibody fragments include, but are not limited to, Fab, F(ab′)₂. Fv fragments, single chain Fv (scFv) antibody fragments, disulfide-linked Fvs, a FD fragment consisting of the VH and CH1 domains, linear antibodies, single domain antibodies (e.g., sdAb (either VH or VL), camelid VHH domains, isolated CDRs, or other epitope-binding fragments of an antibody. Antibody fragments can be incorporated into single domain antibodies, maxibodies, diabodies, minibodies, intrabodies, diabodies, triabodies, tetrabodies, v-NAR, and bis-scFvs, or grafted into polypeptide scaffolds (e.g. fibronectin type III).

The term “chimeric antigen receptor” or “CAR” refers to fusion polypeptide which when present in an immune cell (e.g., a T cell or an NK cell) provides the cell with specificity for a target cell (e.g., a cancer cell) and with intracellular signal generation. In some embodiments, a CAR comprises at least one extracellular domain (e.g., an extracellular antigen-binding domain), a transmembrane domain and a cytoplasmic domain (also referred to herein as an intracellular signaling domain). In some embodiments, the cytoplasmic domain comprises an intracellular signaling domain derived from a stimulatory polypeptide and/or a co-stimulatory polypeptide. In some embodiments, the intracellular signaling domain is derived from the zeta chain associated with the T cell receptor complex (i.e., CD3z), common FcR gamma (FCER1G), 4-1BB, 2B4, DNAX-activating protein 10 (DAP10), DNAX-activating protein 12 (DAP12), OX40, or OX40L. In some embodiments, the CAR comprises an extracellular antigen-binding domain, a transmembrane domain, and a cytoplasmic domain comprising one intracellular signaling domain. In some embodiments, the CAR comprises an extracellular antigen binding domain, a transmembrane domain, and a cytoplasmic domain comprising two intracellular signaling domains. In some embodiments, the CAR comprises a signal peptide (also referred to herein as a leader sequence) at the amino terminus (N-terminus). In some embodiments, the CAR comprises a leader sequence at the N-terminus of an extracellular antigen binding domain. Leader sequences are generally cleaved from the CAR during cellular processing and localization of the CAR to the cellular membrane (e.g., plasma membrane) of the immune cell. In some embodiments, the extracellular antigen-binding domain of the CAR comprises an antibody fragment. In some embodiments, the extracellular antigen-binding domain of a CAR comprises an antibody fragment that is an scFv. In some embodiments, the extracellular antigen-binding domain of a CAR comprises an antibody fragment that is a VHH domain.

The term “signaling domain” as used herein refers to the functional portion of a protein which acts by transmitting information within a cell to regulate a cellular activity (e.g., an effector function) via a signaling pathway (e.g., by generating second messengers) or functioning as an effector (e.g., by responding to a second messenger). A signaling domain can be a co-stimulatory domain and/or an activation domain.

The terms “peptide,” “polypeptide” and “protein” are used interchangeably herein to refer to a compound comprised of amino acid residues covalently linked by peptide bonds.

The term “functional fragment” means a portion of an amino acid sequence (e.g., of a polypeptide) that is substantially identical to, but shorter in length than, a reference polypeptide that retains at least 80%, at least 90%, at least 95%, at least 96%, at least 97%, at least 98%, or at least 99% of one activity (e.g., cognate binding partner (e.g., receptor) binding activity) of the reference polypeptide. A fragment can include an N-terminal truncation, a C-terminal truncation, or both N-terminal and C-terminal truncations relative to a reference polypeptide.

The term “subject” refers to any mammal. In some embodiments, the “subject or “subject in need” of a treatment can be a primate (e.g., a human, a simian (e.g., a monkey (e.g., marmoset or baboon), or an ape (e.g., a gorilla, chimpanzee, orangutan, or gibbon)), a rodent (e.g., a mouse, a guinea pig, a hamster, or a rat), a rabbit, a dog, a cat, a horse, a sheep, a cow, a pig, or a goat. In some embodiments, the subject or “subject suitable for treatment” may be a non-human mammal, especially mammals that are conventionally used as models for demonstrating therapeutic efficacy in humans (e.g., a mouse, a pig, a rat, or a non-human primate) may be employed. In some examples, a subject can be previously diagnosed or identified as being in need of treatment by a medical professional (e.g., a physician, a laboratory technician, a physician's assistant, a nurse, or a clinical laboratory technician).

As used herein, “treating” means a reduction in the number, severity, frequency, and/or duration of one or more symptoms of a medical disease or condition in a subject (e.g., any of the exemplary subjects described herein).

As used herein, “pharmaceutically acceptable carrier” includes any and all aqueous solvents (e.g., saline solutions, phosphate buffered saline, parenteral vehicles, such as sodium chloride, Ringer's dextrose, etc.), antioxidants, preservatives (e.g., antibacterial or antifungal agents, anti-oxidants, chelating agents, and inert gases), isotonic agents, and such like materials and combinations thereof, as would be known to one of ordinary skill in the art. The pH and exact concentration of the various components in a pharmaceutical composition are adjusted according to well-known parameters.

Provided herein are compositions of matter including chimeric antigen receptors (CARs), nucleic acids encoding the CARs, engineered immune cells, pharmaceutical compositions, and kits; methods of treating subjects having a mesothelin-associated cancer; and methods of making engineered immune cells.

### Immune Cells

In one aspect, the disclosure provides a number of CARs comprising an extracellular domain (e.g., an extracellular antigen binding domain) that specifically binds to the protein mesothelin and immune cells that include at least one of the CARs provided herein. In one aspect, the disclosure provides an immune cell (e.g., a T cell or an NK cell) exhibiting an anti-cancer property. The immune cells may be T cells (e.g., regulatory T cells, CD4⁺ T cells, CD8⁺ T cells, or gamma-delta T cells), NK cells, or NKT cells (e.g., invariant NKT cells). The immune cells can be a human, non-human, mammalian, rat, mouse, or dog cell.

In some embodiments, the immune cells comprising a CAR described herein are T cells (e.g., alpha beta T cells and gamma delta T cells). In some embodiments, the T cells are one or more of CD3⁺, CD28⁺, CD4⁺, CD8⁺, CD45RA⁺, CD25⁺ and CD45RO⁺. In some embodiments, the T cells are isolated tumor infiltrating lymphocytes (TIL). In some embodiments, the T cells are CD4⁺ T cells. In some embodiments, the T cells are CD8⁺ T cells. In some embodiments, the T cells are regulatory T cell (e.g., a CD4⁺, CD25⁺, CD62^{hi}, GITR⁻ and FoxP3⁺ T cells). In some embodiments, the T cells are memory T cells (T_{CM}) (e.g., CD62L⁺, CCR7⁺, CD45RO⁻ and CD45RA⁻). In some embodiments, the T cells are stem cell memory T cells. In some embodiments, the T cells are naïve T cells. In some embodiments, the T cells are a mixed population of CD4⁺ T cells, CD8⁺ T cells, stem cell memory T cells and naïve T cells.

In some embodiments, the immune cells comprising a protein described herein (e.g., a CAR) are natural killer T (NKT) cells. NKT cells recognize glycolipid antigen presented by a molecule called CD1d.

In some embodiments, the immune cells comprising a protein described herein (e.g., a CAR) are NK cells. NK cells constitute about 10% of the lymphocytes in human peripheral blood. Human NK cells generally differentiate and mature in the bone marrow, lymph nodes, spleen, tonsils, and thymus. In some embodiments, NK cells are derived from human peripheral blood mononuclear cells (PBMC), unstimulated leukapheresis products (PBSC), human embryonic stem cells (hESCs), induced pluripotent stem cells (iPSCs), mesenchymal stem cells (MSCs), hematopoietic stem cells (HSCs), bone marrow, CD34⁺ cells or umbilical cord blood (CB) by methods well known in the art. In some embodiments, NK cells are isolated from PBMCs. In some embodiments, the NK cells are derived from umbilical CB. The NK cells may be NK cell lines, such as, but not limited to, the NK-92, NK101, KHYG-1, YT, NK-YS, YTS, HANK-1, NKL, and NK3.3 cell lines.

Immune cells provided herein may be expanding using methods known in the art (see e.g., Gregory et al. Methods Mol. Biol. 380:83-105, 2007; Tricket and Kwan, J. Immunol. Methods 275(1-2):251-5, 2003; Schluck et al. Front Immunol. 10:931; Peters et al. Methods Enzymol. 631:223-37, 2020; Andrews et al. Cytotherapy 22(5):276-90; Exley et al. Curr. Protoc. Immunol. 119:14.11.1-14.11.20, 2017; and Becker et al. Cancer Immunol Immunother. 65(4): 477-84). For example, T cells can be expanding by contacting them with a surface having attached thereto an agent that stimulates a CD3/TCR complex-associated signal and a ligand that stimulates a costimulatory molecule on the surface of the T cells, including but not limited to an anti-CD3 antibody or antigen-binding fragment thereof, an anti-CD2 antibody immobilized on a surface, a protein kinase C activator (e.g., bryostatin) in conjunction with a calcium ionophore. In addition, the T cells may also be contacted with a ligand that binds to an accessory molecule on the surface of the T cells (e.g., an anti-CD3 antibody and an anti-CD28 antibody under conditions suitable for the stimulation and proliferation of the T cells.

NK cells can be expanded using methods known in the art. In some instances, NK cells can be expanded or enriched from large volumes of peripheral blood, such as an apheresis products (e.g., mobilized PBSCs or unmobilized PBSCs). In other instances, NK cells can be expanded or enriched from smaller number of blood or stem cells. Expansion of NK cells from apharesis products are described, for example, in Lapteva et al. Crit. Rev. Oncog. 19:121-132, 2014; Miller et al. Blood 105(8):3051-7, 2005; Lapteva et al. Cytotherapy 14(9):1131-43, 2012; Spanholtz et al. PLoS One 6(6):e20740, 2011; Knorr et al. Stem Cells Transl. Med. 2(4):274-83, 2013; Pfeiffer et al. Leukemia 26(11):2435-9, 2012; Shi et al. Br. J. Haematol. 143(5):641-53, 2008; Passweg et al. Leukemia 18(11):1835-8, 2004; Koehl et al. Klin. Padiatr. 217(6):345-50, 2005; and Klingemann et al. Transfusion 53(2):412-8, 2013. NK cells in peripheral blood and apheresis products can be detected by flow cytometry as CD45⁺CD56⁺CD3⁻ cells. In some instances, NK cells can be enriched from apheresis products by one or two rounds of depletion of CD3⁺ T cells using magnetic beads (e.g., CLINIMACS magnetic beads) coated with anti-CD3 antibody (e.g., CLINIMACS CD3 reagent) with or without overnight activation using IL-2 or IL-15. Additional depletion of CD19⁺ B cells with anti-CD19 antibody-coated magnetic beads (e.g., CliniMACS CD19 reagent) can further improve the purity of the NK cells, resulting in an average of 40% CD56⁺CD3⁻ in the final product. Alternatively, NK cells can be enriched by isolating CD56⁺ cells using anti-CD56 monoclonal antibody (e.g., CLINIMACS CD56 reagent) with or without CD3⁺ T cell depletion. In some instances, NK cells can be expanded using feeder cell-based technology. Such methods are described, for example, in Berg et al. Cytotherapy 11(3):341-55, 2009; Lapteva et al. 2012, supra; Lapteva et al. Crit. Rev. Oncog. 19:121-132, 2014, and Yang et al. Mol. Therapy 18:428-445, 2020. Feeder-cell methods generally require cytokines as well as irradiated feeder cells, such as EBV-LCLs or genetically modified K562 cells. CD3-depleted, CD56-enriched PBMCs can be cultured in the presence of EBV-LCL feeders and X-VIVO 20 medium supplemented with 10% heat inactivated human AB serum, 500 U/mL IL-2, and 2 mM L-alanyl-L-glutamine over 21 days of culture (see, e.g., Berg et al. Cytotherapy, 11(3):341-55, 2009).

In some embodiments, the immune cells described herein are NK cells differentiated from stem cells. NK cells can be differentiated from stem cells by various methods known in the art. In some instances, NK cells can be differentiated from induced pluripotent stem cells (iPSCs), human embryonic stem cells (hESCs), mesenchymal stem cells (MSCs), or hematopoietic stem cells (HSCs). Protocols for the differentiation of NK cells from iPSCs and hESCs are described, for example, in Bock et al. J. Vis. Exp. (74):e50337, 2013; Knorr et al. Stem Cells Transi. Med. 2(4):274-83, 2013; Ni et al. Methods Mol. Biol. 1029:33-41, 2013; Zhu and Kaufman Methods Mol. Biol. 2048:107-19, 2019. To differentiate iPSCs to CD34⁺CD45⁺ HPCs, embryonic bodies (EB) can be generated using different approaches, such as spinning of single cell iPSCs in round-shaped wells (spin EBs), culture on murine stroma cells, or direct induction of iPSC monolayer fragments in media with cytokines inducing differentiation towards the hematopoietic lineage. HPCs can be enriched by cell sorting or cell separation of CD34⁺ and/or CD45⁺ cells, and subsequently placed on murine feeder cells (e.g., AFT024, OP9, MS-5, EL08-1D2) in medium containing IL-3 (during the first week), IL-7, IL-15, human stem cell factor (SCF), IL-2, and Flt3L. NK cells can also be differentiated without usage of xenogeneic stromal feeder cells, as described, e.g., by Knorr et al. Stem Cells Transl. Med. 2(4):274-83, 2013. CD3⁻CD56^{bright}CD16^{+/-} NK cells can be differentiated from hiPSC up to stage 4b (NKp80⁺) on OP9-DL1 stroma cells and are highly functional in terms of degranulation, cytokine production and cytotoxicity including antibody-dependent cellular cytotoxicity (ADCC). NK cell yield can be considerably increased through inactivation of feeder cells with mitomycin-C(MMC) without impacting on maturation or functional properties.

Additionally or alternatively, CD56⁺CD16⁺CD3⁻ NK cells can be differentiated from human iPSCs and NK cell development can be characterized by surface expression of NK lineage markers, as described, e.g., by Euchner et al. Front. Immunol. 12:640-672, 2021. Hematopoietic priming of human iPSCs can result in CD34⁺CD45⁺ hematopoietic progenitor cells (HPCs) that do not require enrichment for NK lymphocyte propagation. HPCs can be further differentiated into NK cells on OP9-DL1 feeder cells resulting in high purity of CD56^{bright}CD16⁻ and CD56^{bright} CD16⁺ NK cells. The output of generated NK cells can be increased by inactivating OP9-DL1 feeder cells with MMC. CD7 expression can be detected from the first week of differentiation indicating priming towards the lymphoid lineage. CD56^{bright}CD16-+NK cells expressed high levels of DNAM-1, CD69, natural killer cell receptors NKG2A and NKG2D, and natural cytotoxicity receptors NKp46, NKp44, NKp30. Differentiation of NK cells up to stage 4b can be confirmed by assessing the expression of NKp80 on NK cells, and by a perforin⁺ and granzyme B⁺ phenotype. Differentiation of NK cells can also be confirmed by assessing killer cell immunoglobulin-like receptor KIR2DL2/DL3 and KIR3DL1 on NK cells.

In some instances, CD3⁻CD56⁺ NK cells can be differentiated from CD34⁺ HPCs, as described, e.g., by Cichocki et al. Front Immunol 10: 2078, 2019. NK cell development can occur along a continuum whereby common lymphocyte progenitors (CLPs) gradually downregulate CD34 and upregulate CD56. Acquisition of CD94 marks commitment to the CD56^{bright} stage, and CD56^{bright} NK cells subsequently differentiate into CD56^{dim} NK cells that upregulate CD16 and killer immunoglobulin-like receptors (KIR). Support for this linear model comes from analyses of cell populations in secondary lymphoid tissues and in vitro studies of NK cell development from HPCs.

CD3⁻CD56⁺ NK cells with cytotoxic function can also be differentiated in vitro after long-term culture of CD34⁺ cells isolated from cord blood, bone marrow, fetal liver, thymus, or secondary lymphoid tissue with IL-2 or IL-15, as described, e.g., by Mrozek et al. Blood 87:2632-40, 1996; Jaleco et al. J. Immunol. 159:694-702, 1997; Sanchez et al. J. Ep. Med. 178:1857-66, 1993; and Freud et al. Immunity 22:295-304, 2005.

In certain aspects, the NK cells are isolated and expanded by the previously described method of ex vivo expansion of NK cells (Shah et al. PLoS One 8(10):e76781, 2013). In some embodiments, CB mononuclear cells are isolated by Ficoll density gradient centrifugation and cultured in a bioreactor with IL-2 and artificial antigen presenting cells (aAPCs). After 7 days, the cell culture is depleted of any cells expressing CD3 and re-cultured for an additional 7 days. The cells are again CD3-depleted and characterized to determine the percentage of CD56⁺/CD3⁺ cells or NK cells. In some embodiments, umbilical CB is used to derive NK cells by the isolation of CD34+ cells and differentiation into CD56⁺/CD3⁺ cells by culturing in medium containing SCF, IL-7, IL-15, and IL-2.

Other methods of NK expansion are described in Becker et al., Cancer Immunol. Immunother. 65(4): 477-84, 2016, Phan et al., Methods Mol. Biol. 1441:167-74, 2016, each of which are incorporated herein in reference in their entireties. Commercially available kits for expanding NK cells, such as CellXVivo Human NK Cell Expansion Kit (R&D Systems; Cat. No. CDK015) and NK Cell Activation/Expansion Kit, human (Miltenyi Biotec; Cat No. 130-094-483) can also be used with the methods described herein.

In some embodiments, the immune cells (e.g., T cells and NKT cells) are diaglycerol kinase (DGK)-deficient (e.g., the T cells do not express DGK RNA or protein or have reduced or inhibited DGK activity). In some embodiments, the immune cells (e.g., T cells and NK T cells) are IKAROS-deficient (e.g., the cells do not express IKAROS RNA or protein or have reduced or inhibited IKAROS activity). In some embodiments, the immune cells (e.g., T cells and NKT cells) are both DGK-deficient and IKAROS-deficient.

In some embodiments, the immune cells (e.g., T cells or NK cells) may be selected using techniques well known in the art including positive and negative selection techniques. For example, isolated T cells may be isolated using positive selection by incubation with beads (e.g., magnetic beads) conjugated with anti-CD3 and anti-CD28 antibodies (e.g., Dynabeads® Human T-Expander CD3/CD28 beads; Thermo Fisher Scientific).

In some embodiments, the immune cells (e.g., T cells or NK cells) have been selected based on their expression of one or more of IFN-γ, TNFα, IL-17A, IL-2, IL-3, IL-4, GM-CSF, IL-10, IL-13, granzyme B, and perforin.

The immune cells may be isolated from subjects, particularly human subjects. When a patient is treated with an immune cell including a CAR as described herein, the immune cells may be autologous or allogeneic. In some embodiments, the immune cells have been engineered such that they do not express one or more subunits of a TCR, or express a reduced amount of one or more subunits of a TCR (e.g., as compared to a wild-type immune cell of the same type). In some embodiments, the immune cells have been engineered such that they do not express one or more subunits of an HLA complex (e.g., HLA class I or HLA class II complexes), or express a reduced amount of one or more subunits of an HLA complex (e.g., as compared to a wild-type immune cell of the same type). In some embodiments, the immune cells have been engineered such that they lack expression of a functional TCR and/or a functional HLA complex. Engineered immune cells lacking expression and/or having reduced expression of one or more subunits of a TCR or an HLA complex can be obtained by any suitable means including a knock out or a knock down of one or more subunits of a TCR and/or of an HLA complex.

In some embodiments, the immune cells are from a subject suspected of having, predisposed for, or undergoing treatment for a particular disease or condition (e.g., a disease or condition described herein).

The immune cells may be obtained (e.g., enriched, isolated and/or purified) from any tissue including, but not limited to, blood, spleen, bone marrow, and biopsy tissue. The immune cells may be used after they are obtained (e.g., from a tissue), or can be stored for a period of time prior to use (e.g., cryopreserved). In some embodiments, the immune cells are obtained from blood (e.g., peripheral blood, cord blood (e.g., pooled cord blood). The immune cells may be obtained from a single source or from multiple sources (e.g., 2, 3, 4, 5, 6, 7, 8, 9, 10 or more) sources (e.g., donor subjects).

In some embodiments, the immune cells are genetically engineered to express a CAR provided herein. In some embodiments, the immune cells comprise one or more exogenous nucleic acids introduced via genetic engineering that encode one or more proteins (e.g., a CAR). Suitable methods for genetically engineering immune cells are known in the art (see, e.g., Sambrook et al. Molecular Cloning: A Laboratory Manual, 3rd ed., Cold Spring Harbor Press, Cold Spring Harbor, N.Y., 2001; and Ausubel et al., Current Protocols in Molecular Biology, Greene Publishing Associates and John Wiley & Sons, N Y, 1994). In some embodiments, the immune cells are engineered with a nucleic acid that has been codon-optimized for expression in a mammalian cell (e.g., a human cell).

### Chimeric Antigen Receptors

The present disclosure provides CARs, engineered immune cells (e.g., NK cells, T cells and NKT cells) including the CARs, and nucleic acids encoding the CARs. In some embodiments the engineered immune cells comprise a CAR comprising an amino acid sequence that is at least 80%, at least 85%, at least 90%, at least 95%, at least 96%, at least 97%, at least 98%, at least 99%, or 100% identical to any one of SEQ ID NOs: 1-90. In some embodiments the engineered immune cells comprise a CAR comprising an amino acid sequence that is at least 80%, at least 85%, at least 90%, at least 95%, at least 96%, at least 97%, at least 98%, at least 99%, or 100% identical to any one of the amino acid sequences provided in Table 1.

TABLE 1
Anti-mesothelin CARs

| Exemplary | | |
|---|---|---|
| CAR Name | Amino Acid Sequence | SEQ ID NO |
| AA-MSLN-108 | QVQLVQSGGGLVHPGGSLRLSCAASGIDLSLYRMRWYRQAPGKERDLVALITDD | 1 |
| | GTSYYEDSVKGRFTITRDNPSNKVFLQMNSLKPEDTAVYYCNAETPLSPVNYWG | |
| | QGTQVTVSFVPVFLPAKPTTTPAPRPPTPAPTIASQPLSLRPEACRPAAGGAVH | |
| | TRGLDFACDIYIWAPLAGTCGVLLLSLVITLYCNHRNRKRGRKKLLYIFKQPFM | |
| | RPVQTTQEEDGCSCRFPEEEEGGCELRVKESRSADAPAYQQGQNQLYNELNLGR | |
| | REEYDVLDKRRGRDPEMGGKPRRKNPQEGLYNELQKDKMAEAYSEIGMKGERRR | |
| | GKGHDGLYQGLSTATKDTYDALHMQALPPR | |
| AA-MSLN-109 | QVQLVQSGGGLVQAGGSLRLSCAPSGSIFGIRTMDWYRQAPGKERELVARITMD | 2 |
| | GRVFHADSVKGRFSGSRDGASNAVYLQMNSLKPDDTAVYYCRYSGLTSREDYWG | |
| | PGTQVTVSSFVPVFLPAKPTTTPAPRPPTPAPTIASQPLSLRPEACRPAAGGAV | |
| | HTRGLDFACDIYIWAPLAGTCGVLLLSLVITLYCNHRNRKRGRKKLLYIFKQPF | |
| | MRPVQTTQEEDGCSCRFPEEEEGGCELRVKFSRSADAPAYQQGQNQLYNELNLG | |
| | RREEYDVLDKRRGRDPEMGGKPRRKNPQEGLYNELQKDKMAEAYSEIGMKGERR | |
| | RGKGHDGLYQGLSTATKDTYDALHMQALPPR | |
| AA-MSLN-110 | QVQLVQSGGGLVHPGGSLRLSCAASGIDLSLYRMRWYRQAPGKERDLVALITDD | 3 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRFSGSRDGASNAVYLQMNSL | |
| | KPDDTAVYYCRYSGLTSREDYWGPGTQVTVSSFVPVFLPAKPTTTPAPRPPTPA | |
| | PTIASQPLSLRPEACRPAAGGAVHTRGLDFACDIYIWAPLAGTCGVLLLSLVIT | |
| | LYCNHRNRKRGRKKLLYIFKQPFMRPVQTTQEEDGCSCRFPEEEEGGCELRVKF | |
| | SRSADAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQALPPR | |
| AA-MSLN-111 | QVQLVQSGGGLVHPGGSLRLSCAASGIDLSLYRMRWYRQAPGKERDLVALITDD | 4 |
| | GTSYYEDSVKGRFTITRDNPSNKVFLQMNSLKPEDTAVYYCNAETPLSPVNYWG | |
| | QGTQVTVSESKYGPPCPSCPAPEFEGGPSVFLFPPKPKDTLMISRTPEVTCVVV | |
| | DVSQEDPEVQFNWYVDGVEVHNAKTKPREEQFQSTYRVVSVLTVLHQDWLNGKE | |
| | YKCKVSNKGLPSSIEKTISKAKGQPREPQVYTLPPSQEEMTKNQVSLTCLVKGF | |
| | YPSDIAVEWESNGQPENNYKTTPPVLDSDGSFFLYSRLTVDKSRWQEGNVESCS | |
| | VMHEALHNHYTQKSLSLSLGKIYIWAPLAGTCGVLLLSLVITLYCNHRNRKRGR | |
| | KKLLYIFKQPFMRPVQTTQEEDGCSCRFPEEEEGGCELRVKESRSADAPAYQQG | |
| | QNQLYNELNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| AA-MSLN-112 | QVQLVQSGGGLVHPGGSLRLSCAASGIDLSLYRMRWYRQAPGKERDLVALITDD | 5 |
| | GTSYYEDSVKGRFTITRDNPSNKVFLQMNSLKPEDTAVYYCNAETPLSPVNYWG | |
| | QGTQVTVSFVPVFLPAKPTTTPAPRPPTPAPTIASQPLSLRPEACRPAAGGAVH | |
| | TRGLDFACDFWVLVVVGGVLACYSLLVTVAFIIFWVRKRGRKKLLYIFKQPFMR | |
| | PVQTTQEEDGCSCRFPEEEEGGCELRVKESRSADAPAYQQGQNQLYNELNLGRR | |
| | EEYDVLDKRRGRDPEMGGKPRRKNPQEGLYNELQKDKMAEAYSEIGMKGERRRG | |
| | KGHDGLYQGLSTATKDTYDALHMQALPPR | |
| AA-MSLN-113 | QVQLVQSGGGLVQAGGSLRLSCAPSGSIFGIRTMDWYRQAPGKERELVARITMD | 6 |
| | GRVFHADSVKGRFSGSRDGASNAVYLQMNSLKPDDTAVYYCRYSGLTSREDYWG | |
| | PGTQVTVSSFVPVFLPAKPTTTPAPRPPTPAPTIASQPLSLRPEACRPAAGGAV | |
| | HTRGLDFACDFWVLVVVGGVLACYSLLVTVAFIIFWVRKRGRKKLLYIFKQPFM | |
| | RPVQTTQEEDGCSCRFPEEEEGGCELRVKFSRSADAPAYQQGQNQLYNELNLGR | |
| | REEYDVLDKRRGRDPEMGGKPRRKNPQEGLYNELQKDKMAEAYSEIGMKGERRR | |
| | GKGHDGLYQGLSTATKDTYDALHMQALPPR | |
| AA-MSLN-114 | QVQLVQSGGGLVHPGGSLRLSCAASGIDLSLYRMRWYRQAPGKERDLVALITDD | 7 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRESGSRDGASNAVYLQMNSL | |
| | KPDDTAVYYCRYSGLTSREDYWGPGTQVTVSSFVPVFLPAKPTTTPAPRPPTPA | |
| | PTIASQPLSLRPEACRPAAGGAVHTRGLDFACDFWVLVVVGGVLACYSLLVTVA | |
| | FIIFWVRKRGRKKLLYIFKQPFMRPVQTTQEEDGCSCRFPEEEEGGCELRVKES | |
| | RSADAPAYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQALPPR | |
| AA-MSLN-115 | QVQLVQSGGGLVHPGGSLRLSCAASGIDLSLYRMRWYRQAPGKERDLVALITDD | 8 |
| | GTSYYEDSVKGRFTITRDNPSNKVFLQMNSLKPEDTAVYYCNAETPLSPVNYWG | |
| | QGTQVTVSESKYGPPCPSCPAPEFEGGPSVFLFPPKPKDTLMISRTPEVTCVVV | |
| | DVSQEDPEVQFNWYVDGVEVHNAKTKPREEQFQSTYRVVSVLTVLHQDWLNGKE | |
| | YKCKVSNKGLPSSIEKTISKAKGQPREPQVYTLPPSQEEMTKNQVSLTCLVKGF | |
| | YPSDIAVEWESNGQPENNYKTTPPVLDSDGSFFLYSRLTVDKSRWQEGNVESCS | |
| | VMHEALHNHYTQKSLSLSLGKFWVLVVVGGVLACYSLLVTVAFIIFWVRKRGRK | |
| | KLLYIFKQPFMRPVQTTQEEDGCSCRFPEEEEGGCELRVKFSRSADAPAYQQGQ | |
| | NQLYNELNLGRREEYDVLDKRRGRDPEMGGKPRRKNPQEGLYNELQKDKMAEAY | |
| | SEIGMKGERRRGKGHDGLYQGLSTATKDTYDALHMQALPPR | |
| AA-MSLN-116 | QVQLVQSGGGLVQAGGSLRLSCAPSGSIFGIRTMDWYRQAPGKERELVARITMD | 9 |
| | GRVFHADSVKGRFSGSRDGASNAVYLQMNSLKPDDTAVYYCRYSGLTSREDYWG | |
| | PGTQVTVSSESKYGPPCPSCPAPEFEGGPSVFLFPPKPKDTLMISRTPEVTCVV | |
| | VDVSQEDPEVQFNWYVDGVEVHNAKTKPREEQFQSTYRVVSVLTVLHQDWLNGK | |
| | EYKCKVSNKGLPSSIEKTISKAKGQPREPQVYTLPPSQEEMTKNQVSLTCLVKG | |
| | FYPSDIAVEWESNGQPENNYKTTPPVLDSDGSFFLYSRLTVDKSRWQEGNVESC | |
| | SVMHEALHNHYTQKSLSLSLGKIYIWAPLAGTCGVLLLSLVITLYCNHRNRKRG | |
| | RKKLLYIFKQPFMRPVQTTQEEDGCCRFPEEEEGGCELRVKFSRSADAPAYQQ | |
| | GQNQLYNELNLGRREEYDVLDKRRGRDPEMGGKPRRKNPQEGLYNELQKDKMAE | |
| | AYSEIGMKGERRRGKGHDGLYQGLSTATKDTYDALHMQALPPR | |
| AA-MSLN-117 | QVQLVQSGGGLVHPGGSLRLSCAASGIDLSLYRMRWYRQAPGKERDLVALITDD | 10 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRESGSRDGASNAVYLQMNSL | |
| | KPDDTAVYYCRYSGLTSREDYWGPGTQVTVSSESKYGPPCPSCPAPEFEGGPSV | |
| | FLFPPKPKDTLMISRTPEVTCVVVDVSQEDPEVQFNWYVDGVEVHNAKTKPREE | |
| | QFQSTYRVVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGKIYIWAPLAG | |
| | TCGVLLLSLVITLYCNHRNRKRGRKKLLYIFKQPFMRPVQTTQEEDGCSCREPE | |
| | EEEGGCELRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTATKDT | |
| | YDALHMQALPPR | |
| AA-MSLN-118 | QVQLVQSGGGLVQAGGSLRLSCAPSGSIFGIRTMDWYRQAPGKERELVARITMD | 11 |
| | GRVFHADSVKGRFSGSRDGASNAVYLQMNSLKPDDTAVYYCRYSGLTSREDYWG | |
| | PGTQVTVSSESKYGPPCPSCPAPEFEGGPSVFLFPPKPKDTLMISRTPEVTCVV | |
| | VDVSQEDPEVQFNWYVDGVEVHNAKTKPREEQFQSTYRVVSVLTVLHQDWLNGK | |
| | EYKCKVSNKGLPSSIEKTISKAKGQPREPQVYTLPPSQEEMTKNQVSLTCLVKG | |
| | FYPSDIAVEWESNGQPENNYKTTPPVLDSDGSFFLYSRLTVDKSRWQEGNVFSC | |
| | SVMHEALHNHYTQKSLSLSLGKFWVLVVVGGVLACYSLLVTVAFIIFWVRKRGR | |
| | KKLLYIFKQPFMRPVQTTQEEDGCSCRFPEEEEGGCELRVKFSRSADAPAYQQG | |
| | QNQLYNELNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| AA-MSLN-119 | QVQLVQSGGGLVHPGGSLRLSCAASGIDLSLYRMRWYRQAPGKERDLVALITDD | 12 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRESGSRDGASNAVYLQMNSL | |
| | KPDDTAVYYCRYSGLTSREDYWGPGTQVTVSSESKYGPPCPSCPAPEFEGGPSV | |
| | FLFPPKPKDTLMISRTPEVTCVVVDVSQEDPEVQFNWYVDGVEVHNAKTKPREE | |
| | QFQSTYRVVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGKFWVLVVVGG | |
| | VLACYSLLVTVAFIIFWVRKRGRKKLLYIFKQPFMRPVQTTQEEDGCSCRFPEE | |
| | EEGGCELRVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLSTATKDTY | |
| | DALHMQALPPR | |
| AA-MSLN-120 | QVQLVQSGGGLVHPGGSLRLSCAASGIDLSLYRMRWYRQAPGKERDLVALITDD | 13 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNRKRGRKKLLYIFKQPFMRPVQTTQEEDGCSCRFPEEEEGGCELRVKES | |
| | RSADAPAYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQALPPR | |
| AA-MSLN-121 | QVQLVQSGGGLVQAGGSLRLSCAPSGSIFGIRTMDWYRQAPGKERELVARITMD | 14 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRFSGSRDGASNAVYLQMNS | |
| | LKPDDTAVYYCRYSGLTSREDYWGPGTQVTVSSFVPVFLPAKPTTTPAPRPPTP | |
| | APTIASQPLSLRPEACRPAAGGAVHTRGLDFACDIYIWAPLAGTCGVLLLSLVI | |
| | TLYCNHRNRKRGRKKLLYIFKQPFMRPVQTTQEEDGCSCRFPEEEEGGCELRVK | |
| | FSRSADAPAYQQGQNQLYNELNLGRREEYDVLDKRRGRDPEMGGKPRRKNPQEG | |
| | LYNELQKDKMAEAYSEIGMKGERRRGKGHDGLYQGLSTATKDTYDALHMQALPP | |
| | R | |
| AA-MSLN-122 | QVQLVQSGGGLVHPGGSLRLSCAASGIDLSLYRMRWYRQAPGKERDLVALITDD | 15 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSESKYGPPCPSCPAPEFEGGPSVF | |
| | LFPPKPKDTLMISRTPEVTCVVVDVSQEDPEVQFNWYVDGVEVHNAKTKPREEQ | |
| | FQSTYRVVSVLTVLHQDWLNGKEYKCKVSNKGLPSSIEKTISKAKGQPREPQVY | |
| | TLPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLGKIYIWAPLAGT | |
| | CGVLLLSLVITLYCNHRNRKRGRKKLLYIFKQPFMRPVQTTQEEDGCSCRFPEE | |
| | EEGGCELRVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLSTATKDTY | |
| | DALHMQALPPR | |
| AA-MSLN-123 | QVQLVQSGGGLVQAGGSLRLSCAPSGSIFGIRTMDWYRQAPGKERELVARITMD | 16 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRFSGSRDGASNAVYLQMNS | |
| | LKPDDTAVYYCRYSGLTSREDYWGPGTQVTVSSESKYGPPCPSCPAPEFEGGPS | |
| | VFLFPPKPKDTLMISRTPEVTCVVVDVSQEDPEVQFNWYVDGVEVHNAKTKPRE | |
| | EQFQSTYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPVLDSD | |
| | GSFFLYSRLTVDKSRWQEGNVFSCSVMHEALHNHYTQKSLSLSLGKIYIWAPLA | |
| | GTCGVLLLSLVITLYCNHRNRKRGRKKLLYIFKQPFMRPVQTTQEEDGCSCRFP | |
| | EEEEGGCELRVKFSRSADAPAYQQGQNQLYNELNLGRREEYDVLDKRRGRDPEM | |
| | GGKPRRKNPQEGLYNELQKDKMAEAYSEIGMKGERRRGKGHDGLYQGLSTATKD | |
| | TYDALHMQALPPR | |
| AA-MSLN-124 | QVQLVQSGGGLVHPGGSLRLSCAASGIDLSLYRMRWYRQAPGKERDLVALITDD | 17 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDFWVLVVVGGVLACYSLLVTVAF | |
| | IIFWVRKRGRKKLLYIFKQPFMRPVQTTQEEDGCSCRFPEEEEGGCELRVKFSR | |
| | SADAPAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALPPR | |
| AA-MSLN-125 | QVQLVQSGGGLVQAGGSLRLSCAPSGSIFGIRTMDWYRQAPGKERELVARITMD | 18 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRFSGSRDGASNAVYLQMNS | |
| | LKPDDTAVYYCRYSGLTSREDYWGPGTQVTVSSFVPVFLPAKPTTTPAPRPPTP | |
| | APTIASQPLSLRPEACRPAAGGAVHTRGLDFACDFWVLVVVGGVLACYSLLVTV | |
| | AFIIFWVRKRGRKKLLYIFKQPFMRPVQTTQEEDGCSCRFPEEEEGGCELRVKF | |
| | SRSADAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQALPPR | |
| AA-MSLN-126 | QVQLVQSGGGLVHPGGSLRLSCAASGIDLSLYRMRWYRQAPGKERDLVALITDD | 19 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSESKYGPPCPSCPAPEFEGGPSVF | |
| | LFPPKPKDTLMISRTPEVTCVVVDVSQEDPEVQFNWYVDGVEVHNAKTKPREEQ | |
| | FQSTYRVVSVLTVLHQDWLNGKEYKCKVSNKGLPSSIEKTISKAKGQPREPQVY | |
| | TLPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLGKFWVLVVVGGV | |
| | LACYSLLVTVAFIIFWVRKRGRKKLLYIFKQPFMRPVQTTQEEDGCSCRFPEEE | |
| | EGGCELRVKFSRSADAPAYQQGQNQLYNELNLGRREEYDVLDKRRGRDPEMGGK | |
| | PRRKNPQEGLYNELQKDKMAEAYSEIGMKGERRRGKGHDGLYQGLSTATKDTYD | |
| | ALHMQALPPR | |
| AA-MSLN-127 | QVQLVQSGGGLVQAGGSLRLSCAPSGSIFGIRTMDWYRQAPGKERELVARITMD | 20 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRESGSRDGASNAVYLQMNS | |
| | LKPDDTAVYYCRYSGLTSREDYWGPGTQVTVSSESKYGPPCPSCPAPEFEGGPS | |
| | VFLFPPKPKDTLMISRTPEVTCVVVDVSQEDPEVQFNWYVDGVEVHNAKTKPRE | |
| | EQFQSTYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPVLDSD | |
| | GSFFLYSRLTVDKSRWQEGNVFSCSVMHEALHNHYTQKSLSLSLGKFWVLVVVG | |
| | GVLACYSLLVTVAFIIFWVRKRGRKKLLYIFKQPFMRPVQTTQEEDGCSCRFPE | |
| | EEEGGCELRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTATKDT | |
| | YDALHMQALPPR | |
| AA-MSLN-128 | QVQLVQSGGGLVHPGGSLRLSCAASGIDLSLYRMRWYRQAPGKERDLVALITDD | 21 |
| | GTSYYEDSVKGRFTITRDNPSNKVFLQMNSLKPEDTAVYYCNAETPLSPVNYWG | |
| | QGTQVTVSFVPVFLPAKPTTTPAPRPPTPAPTIASQPLSLRPEACRPAAGGAVH | |
| | TRGLDFACDIYIWAPLAGTCGVLLLSLVITLYCNHRNWRRKRKEKQSETSPKEF | |
| | LTIYEDVKDLKTRRNHEQEQTFPGGGSTIYSMIQSQSSAPTSQEPAYTLYSLIQ | |
| | PSRKSGSRKRNHSPSFNSTIYEVIGKSQPKAQNPARLSRKELENFDVYSRVKES | |
| | RSADAPAYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQALPPR | |
| AA-MSLN-129 | QVQLVQSGGGLVHPGGSLRLSCAASGIDLSLYRMRWYRQAPGKERDLVALITDD | 22 |
| | GTSYYEDSVKGRFTITRDNPSNKVFLQMNSLKPEDTAVYYCNAETPLSPVNYWG | |
| | QGTQVTVSFVPVFLPAKPTTTPAPRPPTPAPTIASQPLSLRPEACRPAAGGAVH | |
| | TRGLDFACDIYIWAPLAGTCGVLLLSLVITLYCNHRNLCARPRRSPAQEDGKVY | |
| | INMPGRGRVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLSTATKDTY | |
| | DALHMQALPPR | |
| AA-MSLN-130 | QVQLVQSGGGLVHPGGSLRLSCAASGIDLSLYRMRWYRQAPGKERDLVALITDD | 23 |
| | GTSYYEDSVKGRFTITRDNPSNKVFLQMNSLKPEDTAVYYCNAETPLSPVNYWG | |
| | QGTQVTVSFVPVFLPAKPTTTPAPRPPTPAPTIASQPLSLRPEACRPAAGGAVH | |
| | TRGLDFACDIYIWAPLAGTCGVLLLSLVITLYCNHRNYFLGRLVPRGRGAAEAA | |
| | TRKQRITETESPYQELQGQRSDVYSDLNTQRPYYKRVKESRSADAPAYQQGQNQ | |
| | LYNELNLGRREEYDVLDKRRGRDPEMGGKPRRKNPQEGLYNELQKDKMAEAYSE | |
| | IGMKGERRRGKGHDGLYQGLSTATKDTYDALHMQALPPR | |
| AA-MSLN-131 | QVQLVQSGGGLVHPGGSLRLSCAASGIDLSLYRMRWYRQAPGKERDLVALITDD | 24 |
| | GTSYYEDSVKGRFTITRDNPSNKVFLQMNSLKPEDTAVYYCNAETPLSPVNYWG | |
| | QGTQVTVSFVPVFLPAKPTTTPAPRPPTPAPTIASQPLSLRPEACRPAAGGAVH | |
| | TRGLDFACDIYIWAPLAGTCGVLLLSLVITLYCNHRNALYLLRRDQRLPPDAHK | |
| | PPGGGSFRTPIQEEQADAHSTLAKIRVKFSRSADAPAYQQGQNQLYNELNLGRR | |
| | EEYDVLDKRRGRDPEMGGKPRRKNPQEGLYNELQKDKMAEAYSEIGMKGERRRG | |
| | KGHDGLYQGLSTATKDTYDALHMQALPPR | |
| AA-MSLN-132 | QVQLVQSGGGLVHPGGSLRLSCAASGIDLSLYRMRWYRQAPGKERDLVALITDD | 25 |
| | GTSYYEDSVKGRFTITRDNPSNKVFLQMNSLKPEDTAVYYCNAETPLSPVNYWG | |
| | QGTQVTVSFVPVFLPAKPTTTPAPRPPTPAPTIASQPLSLRPEACRPAAGGAVH | |
| | TRGLDFACDFWVLVVVGGVLACYSLLVTVAFIIFWVRSKRSRLLHSDYMNMTPR | |
| | RPGPTRKHYQPYAPPRDFAAYRSYFLGRLVPRGRGAAEAATRKQRITETESPYQ | |
| | ELQGQRSDVYSDLNTQRPYYK | |
| AA-MSLN-133 | QVQLVQSGGGLVHPGGSLRLSCAASGIDLSLYRMRWYRQAPGKERDLVALITDD | 26 |
| | GTSYYEDSVKGRFTITRDNPSNKVFLQMNSLKPEDTAVYYCNAETPLSPVNYWG | |
| | QGTQVTVSFVPVFLPAKPTTTPAPRPPTPAPTIASQPLSLRPEACRPAAGGAVH | |
| | TRGLDFACDIYIWAPLAGTCGVLLLSLVITLYCNHRNRKRGRKKLLYIFKQPFM | |
| | RPVQTTQEEDGCSCRFPEEEEGGCELYFLGRLVPRGRGAAEAATRKQRITETES | |
| | PYQELQGQRSDVYSDLNTQRPYYK | |
| AA-MSLN-134 | QVQLVQSGGGLVHPGGSLRLSCAASGIDLSLYRMRWYRQAPGKERDLVALITDD | 27 |
| | GTSYYEDSVKGRFTITRDNPSNKVFLQMNSLKPEDTAVYYCNAETPLSPVNYWG | |
| | QGTQVTVSFVPVFLPAKPTTTPAPRPPTPAPTIASQPLSLRPEACRPAAGGAVH | |
| | TRGLDFACDIYIWAPLAGTCGVLLLSLVITLYCNHRNALYLLRRDQRLPPDAHK | |
| | PPGGGSFRTPIQEEQADAHSTLAKIYFLGRLVPRGRGAAEAATRKQRITETESP | |
| | YQELQGQRSDVYSDLNTQRPYYK | |
| AA-MSLN-135 | QVQLVQSGGGLVHPGGSLRLSCAASGIDLSLYRMRWYRQAPGKERDLVALITDD | 28 |
| | GTSYYEDSVKGRFTITRDNPSNKVFLQMNSLKPEDTAVYYCNAETPLSPVNYWG | |
| | QGTQVTVSFVPVFLPAKPTTTPAPRPPTPAPTIASQPLSLRPEACRPAAGGAVH | |
| | TRGLDFACDFWVLVVVGGVLACYSLLVTVAFIIFWVRSKRSRLLHSDYMNMTPR | |
| | RPGPTRKHYQPYAPPRDFAAYRSRLKIQVRKAAITSYEKSDGVYTGLSTRNQET | |
| | YETLKHEKPPQ | |
| AA-MSLN-136 | QVQLVQSGGGLVHPGGSLRLSCAASGIDLSLYRMRWYRQAPGKERDLVALITDD | 29 |
| | GTSYYEDSVKGRFTITRDNPSNKVFLQMNSLKPEDTAVYYCNAETPLSPVNYWG | |
| | QGTQVTVSFVPVFLPAKPTTTPAPRPPTPAPTIASQPLSLRPEACRPAAGGAVH | |
| | TRGLDFACDIYIWAPLAGTCGVLLLSLVITLYCNHRNRVKESRSADAPAYQQGQ | |
| | NQLYNELNLGRREEYDVLDKRRGRDPEMGGKPRRKNPQEGLYNELQKDKMAEAY | |
| | SEIGMKGERRRGKGHDGLYQGLSTATKDTYDALHMQALPPR | |
| AA-MSLN-137 | QVQLVQSGGGLVHPGGSLRLSCAASGIDLSLYRMRWYRQAPGKERDLVALITDD | 30 |
| | GTSYYEDSVKGRFTITRDNPSNKVFLQMNSLKPEDTAVYYCNAETPLSPVNYWG | |
| | QGTQVTVSFVPVFLPAKPTTTPAPRPPTPAPTIASQPLSLRPEACRPAAGGAVH | |
| | TRGLDFACDFWVLVVVGGVLACYSLLVTVAFIIFWVRSKRSRLLHSDYMNMTPR | |
| | RPGPTRKHYQPYAPPRDFAAYRSRVKFSRSADAPAYQQGQNQLYNELNLGRREE | |
| | YDVLDKRRGRDPEMGGKPRRKNPQEGLYNELQKDKMAEAYSEIGMKGERRRGKG | |
| | HDGLYQGLSTATKDTYDALHMQALPPR | |
| AA-MSLN-138 | QVQLVQSGGGLVHPGGSLRLSCAASGIDLSLYRMRWYRQAPGKERDLVALITDD | 31 |
| | GTSYYEDSVKGRFTITRDNPSNKVELQMNSLKPEDTAVYYCNAETPLSPVNYWG | |
| | QGTQVTVSFVPVFLPAKPTTTPAPRPPTPAPTIASQPLSLRPEACRPAAGGAVH | |
| | TRGLDFACDFWVLVVVGGVLACYSLLVTVAFIIFWVRSKRSRLLHSDYMNMTPR | |
| | RPGPTRKHYQPYAPPRDFAAYRSERVQPLEENVGNAARPRFERNKRVKFSRSAD | |
| | APAYQQGQNQLYNELNLGRREEYDVLDKRRGRDPEMGGKPRRKNPQEGLYNELQ | |
| | KDKMAEAYSEIGMKGERRRGKGHDGLYQGLSTATKDTYDALHMQALPPR | |
| AA-MSLN-139 | QVQLVQSGGGLVQAGGSLRLSCAPSGSIFGIRTMDWYRQAPGKERELVARITMD | 32 |
| | GRVFHADSVKGRFSGSRDGASNAVYLQMNSLKPDDTAVYYCRYSGLTSREDYWG | |
| | PGTQVTVSSFVPVFLPAKPTTTPAPRPPTPAPTIASQPLSLRPEACRPAAGGAV | |
| | HTRGLDFACDIYIWAPLAGTCGVLLLSLVITLYCNHRNWRRKRKEKQSETSPKE | |
| | FLTIYEDVKDLKTRRNHEQEQTFPGGGSTIYSMIQSQSSAPTSQEPAYTLYSLI | |
| | QPSRKSGSRKRNHSPSFNSTIYEVIGKSQPKAQNPARLSRKELENFDVYSRVKF | |
| | SRSADAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQALPPR | |
| AA-MSLN-140 | QVQLVQSGGGLVQAGGSLRLSCAPSGSIFGIRTMDWYRQAPGKERELVARITMD | 33 |
| | GRVFHADSVKGRFSGSRDGASNAVYLQMNSLKPDDTAVYYCRYSGLTSREDYWG | |
| | PGTQVTVSSFVPVFLPAKPTTTPAPRPPTPAPTIASQPLSLRPEACRPAAGGAV | |
| | HTRGLDFACDIYIWAPLAGTCGVLLLSLVITLYCNHRNLCARPRRSPAQEDGKV | |
| | YINMPGRGRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTATKDT | |
| | YDALHMQALPPR | |
| AA-MSLN-141 | QVQLVQSGGGLVQAGGSLRLSCAPSGSIFGIRTMDWYRQAPGKERELVARITMD | 34 |
| | GRVFHADSVKGRFSGSRDGASNAVYLQMNSLKPDDTAVYYCRYSGLTSREDYWG | |
| | PGTQVTVSSFVPVFLPAKPTTTPAPRPPTPAPTIASQPLSLRPEACRPAAGGAV | |
| | HTRGLDFACDIYIWAPLAGTCGVLLLSLVITLYCNHRNYFLGRLVPRGRGAAEA | |
| | ATRKQRITETESPYQELQGQRSDVYSDLNTQRPYYKRVKFSRSADAPAYQQGQN | |
| | QLYNELNLGRREEYDVLDKRRGRDPEMGGKPRRKNPQEGLYNELQKDKMAEAYS | |
| | EIGMKGERRRGKGHDGLYQGLSTATKDTYDALHMQALPPR | |
| AA-MSLN-142 | QVQLVQSGGGLVQAGGSLRLSCAPSGSIFGIRTMDWYRQAPGKERELVARITMD | 35 |
| | GRVFHADSVKGRFSGSRDGASNAVYLQMNSLKPDDTAVYYCRYSGLTSREDYWG | |
| | PGTQVTVSSFVPVFLPAKPTTTPAPRPPTPAPTIASQPLSLRPEACRPAAGGAV | |
| | HTRGLDFACDIYIWAPLAGTCGVLLLSLVITLYCNHRNALYLLRRDQRLPPDAH | |
| | KPPGGGSFRTPIQEEQADAHSTLAKIRVKFSRSADAPAYQQGQNQLYNELNLGR | |
| | REEYDVLDKRRGRDPEMGGKPRRKNPQEGLYNELQKDKMAEAYSEIGMKGERRR | |
| | GKGHDGLYQGLSTATKDTYDALHMQALPPR | |
| AA-MSLN-143 | QVQLVQSGGGLVQAGGSLRLSCAPSGSIFGIRTMDWYRQAPGKERELVARITMD | 36 |
| | GRVFHADSVKGRFSGSRDGASNAVYLQMNSLKPDDTAVYYCRYSGLTSREDYWG | |
| | PGTQVTVSSFVPVFLPAKPTTTPAPRPPTPAPTIASQPLSLRPEACRPAAGGAV | |
| | HTRGLDFACDFWVLVVVGGVLACYSLLVTVAFIIFWVRSKRSRLLHSDYMNMTP | |
| | RRPGPTRKHYQPYAPPRDFAAYRSYFLGRLVPRGRGAAEAATRKQRITETESPY | |
| | QELQGQRSDVYSDLNTQRPYYK | |
| AA-MSLN-144 | QVQLVQSGGGLVQAGGSLRLSCAPSGSIFGIRTMDWYRQAPGKERELVARITMD | 37 |
| | GRVFHADSVKGRFSGSRDGASNAVYLQMNSLKPDDTAVYYCRYSGLTSREDYWG | |
| | PGTQVTVSSFVPVFLPAKPTTTPAPRPPTPAPTIASQPLSLRPEACRPAAGGAV | |
| | HTRGLDFACDIYIWAPLAGTCGVLLLSLVITLYCNHRNRKRGRKKLLYIFKQPF | |
| | MRPVQTTQEEDGCSCRFPEEEEGGCELYFLGRLVPRGRGAAEAATRKQRITETE | |
| | SPYQELQGQRSDVYSDLNTQRPYYK | |
| AA-MSLN-145 | QVQLVQSGGGLVQAGGSLRLSCAPSGSIFGIRTMDWYRQAPGKERELVARITMD | 38 |
| | GRVFHADSVKGRFSGSRDGASNAVYLQMNSLKPDDTAVYYCRYSGLTSREDYWG | |
| | PGTQVTVSSFVPVFLPAKPTTTPAPRPPTPAPTIASQPLSLRPEACRPAAGGAV | |
| | HTRGLDFACDIYIWAPLAGTCGVLLLSLVITLYCNHRNALYLLRRDQRLPPDAH | |
| | KPPGGGSFRTPIQEEQADAHSTLAKIYFLGRLVPRGRGAAEAATRKQRITETES | |
| | PYQELQGQRSDVYSDLNTQRPYYK | |
| AA-MSLN-146 | QVQLVQSGGGLVQAGGSLRLSCAPSGSIFGIRTMDWYRQAPGKERELVARITMD | 39 |
| | GRVFHADSVKGRESGSRDGASNAVYLQMNSLKPDDTAVYYCRYSGLTSREDYWG | |
| | PGTQVTVSSFVPVFLPAKPTTTPAPRPPTPAPTIASQPLSLRPEACRPAAGGAV | |
| | HTRGLDFACDFWVLVVVGGVLACYSLLVTVAFIIFWVRSKRSRLLHSDYMNMTP | |
| | RRPGPTRKHYQPYAPPRDFAAYRSRLKIQVRKAAITSYEKSDGVYTGLSTRNQE | |
| | TYETLKHEKPPQ | |
| AA-MSLN-147 | QVQLVQSGGGLVQAGGSLRLSCAPSGSIFGIRTMDWYRQAPGKERELVARITMD | 40 |
| | GRVFHADSVKGRFSGSRDGASNAVYLQMNSLKPDDTAVYYCRYSGLTSREDYWG | |
| | PGTQVTVSSFVPVFLPAKPTTTPAPRPPTPAPTIASQPLSLRPEACRPAAGGAV | |
| | HTRGLDFACDIYIWAPLAGTCGVLLLSLVITLYCNHRNRVKFSRSADAPAYQQG | |
| | QNQLYNELNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| AA-MSLN-148 | QVQLVQSGGGLVQAGGSLRLSCAPSGSIFGIRTMDWYRQAPGKERELVARITMD | 41 |
| | GRVFHADSVKGRFSGSRDGASNAVYLQMNSLKPDDTAVYYCRYSGLTSREDYWG | |
| | PGTQVTVSSFVPVFLPAKPTTTPAPRPPTPAPTIASQPLSLRPEACRPAAGGAV | |
| | HTRGLDFACDFWVLVVVGGVLACYSLLVTVAFIIFWVRSKRSRLLHSDYMNMTP | |
| | RRPGPTRKHYQPYAPPRDFAAYRSRVKFSRSADAPAYQQGQNQLYNELNLGRRE | |
| | EYDVLDKRRGRDPEMGGKPRRKNPQEGLYNELQKDKMAEAYSEIGMKGERRRGK | |
| | GHDGLYQGLSTATKDTYDALHMQALPPR | |
| AA-MSLN-149 | QVQLVQSGGGLVQAGGSLRLSCAPSGSIFGIRTMDWYRQAPGKERELVARITMD | 42 |
| | GRVFHADSVKGRFSGSRDGASNAVYLQMNSLKPDDTAVYYCRYSGLTSREDYWG | |
| | PGTQVTVSSFVPVFLPAKPTTTPAPRPPTPAPTIASQPLSLRPEACRPAAGGAV | |
| | HTRGLDFACDFWVLVVVGGVLACYSLLVTVAFIIFWVRSKRSRLLHSDYMNMTP | |
| | RRPGPTRKHYQPYAPPRDFAAYRSERVQPLEENVGNAARPRFERNKRVKESRSA | |
| | DAPAYQQGQNQLYNELNLGRREEYDVLDKRRGRDPEMGGKPRRKNPQEGLYNEL | |
| | QKDKMAEAYSEIGMKGERRRGKGHDGLYQGLSTATKDTYDALHMQALPPR | |
| AA-MSLN-150 | QVQLVQSGGGLVHPGGSLRLSCAASGIDLSLYRMRWYRQAPGKERDLVALITDD | 43 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRESGSRDGASNAVYLQMNSL | |
| | KPDDTAVYYCRYSGLTSREDYWGPGTQVTVSSFVPVFLPAKPTTTPAPRPPTPA | |
| | PTIASQPLSLRPEACRPAAGGAVHTRGLDFACDIYIWAPLAGTCGVLLLSLVIT | |
| | LYCNHRNWRRKRKEKQSETSPKEFLTIYEDVKDLKTRRNHEQEQTFPGGGSTIY | |
| | SMIQSQSSAPTSQEPAYTLYSLIQPSRKSGSRKRNHSPSFNSTIYEVIGKSQPK | |
| | AQNPARLSRKELENFDVYSRVKFSRSADAPAYQQGQNQLYNELNLGRREEYDVL | |
| | DKRRGRDPEMGGKPRRKNPQEGLYNELQKDKMAEAYSEIGMKGERRRGKGHDGL | |
| | YQGLSTATKDTYDALHMQALPPR | |
| AA-MSLN-151 | QVQLVQSGGGLVHPGGSLRLSCAASGIDLSLYRMRWYRQAPGKERDLVALITDD | 44 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRFSGSRDGASNAVYLQMNSL | |
| | KPDDTAVYYCRYSGLTSREDYWGPGTQVTVSSFVPVFLPAKPTTTPAPRPPTPA | |
| | PTIASQPLSLRPEACRPAAGGAVHTRGLDFACDIYIWAPLAGTCGVLLLSLVIT | |
| | LYCNHRNLCARPRRSPAQEDGKVYINMPGRGRVKFSRSADAPAYQQGQNQLYNE | |
| | LNLGRREEYDVLDKRRGRDPEMGGKPRRKNPQEGLYNELQKDKMAEAYSEIGMK | |
| | GERRRGKGHDGLYQGLSTATKDTYDALHMQALPPR | |
| AA-MSLN-152 | QVQLVQSGGGLVHPGGSLRLSCAASGIDLSLYRMRWYRQAPGKERDLVALITDD | 45 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRESGSRDGASNAVYLQMNSL | |
| | KPDDTAVYYCRYSGLTSREDYWGPGTQVTVSSFVPVELPAKPTTTPAPRPPTPA | |
| | PTIASQPLSLRPEACRPAAGGAVHTRGLDFACDIYIWAPLAGTCGVLLLSLVIT | |
| | LYCNHRNYFLGRLVPRGRGAAEAATRKQRITETESPYQELQGQRSDVYSDLNTQ | |
| | RPYYKRVKFSRSADAPAYQQGQNQLYNELNLGRREEYDVLDKRRGRDPEMGGKP | |
| | RRKNPQEGLYNELQKDKMAEAYSEIGMKGERRRGKGHDGLYQGLSTATKDTYDA | |
| | LHMQALPPR | |
| AA-MSLN-153 | QVQLVQSGGGLVHPGGSLRLSCAASGIDLSLYRMRWYRQAPGKERDLVALITDD | 46 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRFSGSRDGASNAVYLQMNSL | |
| | KPDDTAVYYCRYSGLTSREDYWGPGTQVTVSSFVPVFLPAKPTTTPAPRPPTPA | |
| | PTIASQPLSLRPEACRPAAGGAVHTRGLDFACDIYIWAPLAGTCGVLLLSLVIT | |
| | LYCNHRNALYLLRRDQRLPPDAHKPPGGGSFRTPIQEEQADAHSTLAKIRVKES | |
| | RSADAPAYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQALPPR | |
| AA-MSLN-154 | QVQLVQSGGGLVHPGGSLRLSCAASGIDLSLYRMRWYRQAPGKERDLVALITDD | 47 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRFSGSRDGASNAVYLQMNSL | |
| | KPDDTAVYYCRYSGLTSREDYWGPGTQVTVSSFVPVFLPAKPTTTPAPRPPTPA | |
| | PTIASQPLSLRPEACRPAAGGAVHTRGLDFACDFWVLVVVGGVLACYSLLVTVA | |
| | FIIFWVRSKRSRLLHSDYMNMTPRRPGPTRKHYQPYAPPRDFAAYRSYFLGRLV | |
| | PRGRGAAEAATRKQRITETESPYQELQGQRSDVYSDLNTQRPYYK | |
| AA-MSLN-155 | QVQLVQSGGGLVHPGGSLRLSCAASGIDLSLYRMRWYRQAPGKERDLVALITDD | 48 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRFSGSRDGASNAVYLQMNSL | |
| | KPDDTAVYYCRYSGLTSREDYWGPGTQVTVSSFVPVFLPAKPTTTPAPRPPTPA | |
| | PTIASQPLSLRPEACRPAAGGAVHTRGLDFACDIYIWAPLAGTCGVLLLSLVIT | |
| | LYCNHRNRKRGRKKLLYIFKQPFMRPVQTTQEEDGCSCRFPEEEEGGCELYFLG | |
| | RLVPRGRGAAEAATRKQRITETESPYQELQGQRSDVYSDLNTQRPYYK | |
| AA-MSLN-156 | QVQLVQSGGGLVHPGGSLRLSCAASGIDLSLYRMRWYRQAPGKERDLVALITDD | 49 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRFSGSRDGASNAVYLQMNSL | |
| | KPDDTAVYYCRYSGLTSREDYWGPGTQVTVSSFVPVFLPAKPTTTPAPRPPTPA | |
| | PTIASQPLSLRPEACRPAAGGAVHTRGLDFACDIYIWAPLAGTCGVLLLSLVIT | |
| | LYCNHRNALYLLRRDQRLPPDAHKPPGGGSFRTPIQEEQADAHSTLAKIYFLGR | |
| | LVPRGRGAAEAATRKQRITETESPYQELQGQRSDVYSDLNTQRPYYK | |
| AA-MSLN-157 | QVQLVQSGGGLVHPGGSLRLSCAASGIDLSLYRMRWYRQAPGKERDLVALITDD | 50 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRFSGSRDGASNAVYLQMNSL | |
| | KPDDTAVYYCRYSGLTSREDYWGPGTQVTVSSFVPVELPAKPTTTPAPRPPTPA | |
| | PTIASQPLSLRPEACRPAAGGAVHTRGLDFACDFWVLVVVGGVLACYSLLVTVA | |
| | FIIFWVRSKRSRLLHSDYMNMTPRRPGPTRKHYQPYAPPRDFAAYRSRLKIQVR | |
| | KAAITSYEKSDGVYTGLSTRNQETYETLKHEKPPQ | |
| AA-MSLN-158 | QVQLVQSGGGLVHPGGSLRLSCAASGIDLSLYRMRWYRQAPGKERDLVALITDD | 51 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRESGSRDGASNAVYLQMNSL | |
| | KPDDTAVYYCRYSGLTSREDYWGPGTQVTVSSFVPVFLPAKPTTTPAPRPPTPA | |
| | PTIASQPLSLRPEACRPAAGGAVHTRGLDFACDIYIWAPLAGTCGVLLLSLVIT | |
| | LYCNHRNRVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLSTATKDTY | |
| | DALHMQALPPR | |
| AA-MSLN-159 | QVQLVQSGGGLVHPGGSLRLSCAASGIDLSLYRMRWYRQAPGKERDLVALITDD | 52 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRFSGSRDGASNAVYLQMNSL | |
| | KPDDTAVYYCRYSGLTSREDYWGPGTQVTVSSFVPVFLPAKPTTTPAPRPPTPA | |
| | PTIASQPLSLRPEACRPAAGGAVHTRGLDFACDFWVLVVVGGVLACYSLLVTVA | |
| | FIIFWVRSKRSRLLHSDYMNMTPRRPGPTRKHYQPYAPPRDFAAYRSRVKFSRS | |
| | ADAPAYQQGQNQLYNELNLGRREEYDVLDKRRGRDPEMGGKPRRKNPQEGLYNE | |
| | LQKDKMAEAYSEIGMKGERRRGKGHDGLYQGLSTATKDTYDALHMQALPPR | |
| AA-MSLN-160 | QVQLVQSGGGLVHPGGSLRLSCAASGIDLSLYRMRWYRQAPGKERDLVALITDD | 53 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVQAGGSLRLSCAPSGSIFGI | |
| | RTMDWYRQAPGKERELVARITMDGRVFHADSVKGRESGSRDGASNAVYLQMNSL | |
| | KPDDTAVYYCRYSGLTSREDYWGPGTQVTVSSFVPVFLPAKPTTTPAPRPPTPA | |
| | PTIASQPLSLRPEACRPAAGGAVHTRGLDFACDFWVLVVVGGVLACYSLLVTVA | |
| | FIIFWVRSKRSRLLHSDYMNMTPRRPGPTRKHYQPYAPPRDFAAYRSERVQPLE | |
| | ENVGNAARPRFERNKRVKFSRSADAPAYQQGQNQLYNELNLGRREEYDVLDKRR | |
| | GRDPEMGGKPRRKNPQEGLYNELQKDKMAEAYSEIGMKGERRRGKGHDGLYQGL | |
| | STATKDTYDALHMQALPPR | |
| AA-MSLN-161 | QVQLVQSGGGLVHPGGSLRLSCAASGIDLSLYRMRWYRQAPGKERDLVALITDD | 54 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNWRRKRKEKQSETSPKEFLTIYEDVKDLKTRRNHEQEQTFPGGGSTIYS | |
| | MIQSQSSAPTSQEPAYTLYSLIQPSRKSGSRKRNHSPSENSTIYEVIGKSQPKA | |
| | QNPARLSRKELENFDVYSRVKFSRSADAPAYQQGQNQLYNELNLGRREEYDVLD | |
| | KRRGRDPEMGGKPRRKNPQEGLYNELQKDKMAEAYSEIGMKGERRRGKGHDGLY | |
| | QGLSTATKDTYDALHMQALPPR | |
| AA-MSLN-162 | QVQLVQSGGGLVHPGGSLRLSCAASGIDLSLYRMRWYRQAPGKERDLVALITDD | 55 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNLCARPRRSPAQEDGKVYINMPGRGRVKFSRSADAPAYQQGQNQLYNEL | |
| | NLGRREEYDVLDKRRGRDPEMGGKPRRKNPQEGLYNELQKDKMAEAYSEIGMKG | |
| | ERRRGKGHDGLYQGLSTATKDTYDALHMQALPPR | |
| AA-MSLN-163 | QVQLVQSGGGLVHPGGSLRLSCAASGIDLSLYRMRWYRQAPGKERDLVALITDD | 56 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNYFLGRLVPRGRGAAEAATRKQRITETESPYQELQGQRSDVYSDLNTQR | |
| | PYYKRVKFSRSADAPAYQQGQNQLYNELNLGRREEYDVLDKRRGRDPEMGGKPR | |
| | RKNPQEGLYNELQKDKMAEAYSEIGMKGERRRGKGHDGLYQGLSTATKDTYDAL | |
| | HMQALPPR | |
| AA-MSLN-164 | QVQLVQSGGGLVHPGGSLRLSCAASGIDLSLYRMRWYRQAPGKERDLVALITDD | 57 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNALYLLRRDQRLPPDAHKPPGGGSFRTPIQEEQADAHSTLAKIRVKFSR | |
| | SADAPAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALPPR | |
| AA-MSLN-165 | QVQLVQSGGGLVHPGGSLRLSCAASGIDLSLYRMRWYRQAPGKERDLVALITDD | 58 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDFWVLVVVGGVLACYSLLVTVAF | |
| | IIFWVRSKRSRLLHSDYMNMTPRRPGPTRKHYQPYAPPRDFAAYRSYFLGRLVP | |
| | RGRGAAEAATRKQRITETESPYQELQGQRSDVYSDLNTQRPYYK | |
| AA-MSLN-166 | QVQLVQSGGGLVHPGGSLRLSCAASGIDLSLYRMRWYRQAPGKERDLVALITDD | 59 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVELQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNRKRGRKKLLYIFKQPFMRPVQTTQEEDGCSCRFPEEEEGGCELYFLGR | |
| | LVPRGRGAAEAATRKQRITETESPYQELQGQRSDVYSDLNTQRPYYK | |
| AA-MSLN-167 | QVQLVQSGGGLVHPGGSLRLSCAASGIDLSLYRMRWYRQAPGKERDLVALITDD | 60 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNALYLLRRDQRLPPDAHKPPGGGSFRTPIQEEQADAHSTLAKIYFLGRL | |
| | VPRGRGAAEAATRKQRITETESPYQELQGQRSDVYSDLNTQRPYYK | |
| AA-MSLN-168 | QVQLVQSGGGLVHPGGSLRLSCAASGIDLSLYRMRWYRQAPGKERDLVALITDD | 61 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDFWVLVVVGGVLACYSLLVTVAF | |
| | IIFWVRSKRSRLLHSDYMNMTPRRPGPTRKHYQPYAPPRDFAAYRSRLKIQVRK | |
| | AAITSYEKSDGVYTGLSTRNQETYETLKHEKPPQ | |
| AA-MSLN-169 | QVQLVQSGGGLVHPGGSLRLSCAASGIDLSLYRMRWYRQAPGKERDLVALITDD | 62 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNRVKFSRSADAPAYQQGQNQLYNELNLGRREEYDVLDKRRGRDPEMGGK | |
| | PRRKNPQEGLYNELQKDKMAEAYSEIGMKGERRRGKGHDGLYQGLSTATKDTYD | |
| | ALHMQALPPR | |
| AA-MSLN-170 | QVQLVQSGGGLVHPGGSLRLSCAASGIDLSLYRMRWYRQAPGKERDLVALITDD | 63 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDFWVLVVVGGVLACYSLLVTVAF | |
| | IIFWVRSKRSRLLHSDYMNMTPRRPGPTRKHYQPYAPPRDFAAYRSRVKFSRSA | |
| | DAPAYQQGQNQLYNELNLGRREEYDVLDKRRGRDPEMGGKPRRKNPQEGLYNEL | |
| | QKDKMAEAYSEIGMKGERRRGKGHDGLYQGLSTATKDTYDALHMQALPPR | |
| AA-MSLN-171 | QVQLVQSGGGLVHPGGSLRLSCAASGIDLSLYRMRWYRQAPGKERDLVALITDD | 64 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDFWVLVVVGGVLACYSLLVTVAF | |
| | IIFWVRSKRSRLLHSDYMNMTPRRPGPTRKHYQPYAPPRDFAAYRSERVQPLEE | |
| | NVGNAARPRFERNKRVKFSRSADAPAYQQGQNQLYNELNLGRREEYDVLDKRRG | |
| | RDPEMGGKPRRKNPQEGLYNELQKDKMAEAYSEIGMKGERRRGKGHDGLYQGLS | |
| | TATKDTYDALHMQALPPR | |
| AA-MSLN-172 | QVQLVQSGGGLVQAGGSLRLSCAPSGSIFGIRTMDWYRQAPGKERELVARITMD | 65 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRFSGSRDGASNAVYLQMNS | |
| | LKPDDTAVYYCRYSGLTSREDYWGPGTQVTVSSFVPVFLPAKPTTTPAPRPPTP | |
| | APTIASQPLSLRPEACRPAAGGAVHTRGLDFACDIYIWAPLAGTCGVLLLSLVI | |
| | TLYCNHRNWRRKRKEKQSETSPKEFLTIYEDVKDLKTRRNHEQEQTFPGGGSTI | |
| | YSMIQSQSSAPTSQEPAYTLYSLIQPSRKSGSRKRNHSPSFNSTIYEVIGKSQP | |
| | KAQNPARLSRKELENFDVYSRVKFSRSADAPAYQQGQNQLYNELNLGRREEYDV | |
| | LDKRRGRDPEMGGKPRRKNPQEGLYNELQKDKMAEAYSEIGMKGERRRGKGHDG | |
| | LYQGLSTATKDTYDALHMQALPPR | |
| AA-MSLN-173 | QVQLVQSGGGLVQAGGSLRLSCAPSGSIFGIRTMDWYRQAPGKERELVARITMD | 66 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRFSGSRDGASNAVYLQMNS | |
| | LKPDDTAVYYCRYSGLTSREDYWGPGTQVTVSSFVPVFLPAKPTTTPAPRPPTP | |
| | APTIASQPLSLRPEACRPAAGGAVHTRGLDFACDIYIWAPLAGTCGVLLLSLVI | |
| | TLYCNHRNLCARPRRSPAQEDGKVYINMPGRGRVKFSRSADAPAYQQGQNQLYN | |
| | ELNLGRREEYDVLDKRRGRDPEMGGKPRRKNPQEGLYNELQKDKMAEAYSEIGM | |
| | KGERRRGKGHDGLYQGLSTATKDTYDALHMQALPPR | |
| AA-MSLN-174 | QVQLVQSGGGLVQAGGSLRLSCAPSGSIFGIRTMDWYRQAPGKERELVARITMD | 67 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRESGSRDGASNAVYLQMNS | |
| | LKPDDTAVYYCRYSGLTSREDYWGPGTQVTVSSFVPVFLPAKPTTTPAPRPPTP | |
| | APTIASQPLSLRPEACRPAAGGAVHTRGLDFACDIYIWAPLAGTCGVLLLSLVI | |
| | TLYCNHRNYFLGRLVPRGRGAAEAATRKQRITETESPYQELQGQRSDVYSDLNT | |
| | QRPYYKRVKFSRSADAPAYQQGQNQLYNELNLGRREEYDVLDKRRGRDPEMGGK | |
| | PRRKNPQEGLYNELQKDKMAEAYSEIGMKGERRRGKGHDGLYQGLSTATKDTYD | |
| | ALHMQALPPR | |
| AA-MSLN-175 | QVQLVQSGGGLVQAGGSLRLSCAPSGSIFGIRTMDWYRQAPGKERELVARITMD | 68 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRFSGSRDGASNAVYLQMNS | |
| | LKPDDTAVYYCRYSGLTSREDYWGPGTQVTVSSFVPVFLPAKPTTTPAPRPPTP | |
| | APTIASQPLSLRPEACRPAAGGAVHTRGLDFACDIYIWAPLAGTCGVLLLSLVI | |
| | TLYCNHRNALYLLRRDQRLPPDAHKPPGGGSFRTPIQEEQADAHSTLAKIRVKF | |
| | SRSADAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQALPPR | |
| AA-MSLN-176 | QVQLVQSGGGLVQAGGSLRLSCAPSGSIFGIRTMDWYRQAPGKERELVARITMD | 69 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRFSGSRDGASNAVYLQMNS | |
| | LKPDDTAVYYCRYSGLTSREDYWGPGTQVTVSSFVPVFLPAKPTTTPAPRPPTP | |
| | APTIASQPLSLRPEACRPAAGGAVHTRGLDFACDFWVLVVVGGVLACYSLLVTV | |
| | AFIIFWVRSKRSRLLHSDYMNMTPRRPGPTRKHYQPYAPPRDFAAYRSYFLGRL | |
| | VPRGRGAAEAATRKQRITETESPYQELQGQRSDVYSDLNTQRPYYK | |
| AA-MSLN-177 | QVQLVQSGGGLVQAGGSLRLSCAPSGSIFGIRTMDWYRQAPGKERELVARITMD | 70 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRFSGSRDGASNAVYLQMNS | |
| | LKPDDTAVYYCRYSGLTSREDYWGPGTQVTVSSFVPVFLPAKPTTTPAPRPPTP | |
| | APTIASQPLSLRPEACRPAAGGAVHTRGLDFACDIYIWAPLAGTCGVLLLSLVI | |
| | TLYCNHRNRKRGRKKLLYIFKQPFMRPVQTTQEEDGCSCRFPEEEEGGCELYFL | |
| | GRLVPRGRGAAEAATRKQRITETESPYQELQGQRSDVYSDLNTQRPYYK | |
| AA-MSLN-178 | QVQLVQSGGGLVQAGGSLRLSCAPSGSIFGIRTMDWYRQAPGKERELVARITMD | 71 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRFSGSRDGASNAVYLQMNS | |
| | LKPDDTAVYYCRYSGLTSREDYWGPGTQVTVSSFVPVFLPAKPTTTPAPRPPTP | |
| | APTIASQPLSLRPEACRPAAGGAVHTRGLDFACDIYIWAPLAGTCGVLLLSLVI | |
| | TLYCNHRNALYLLRRDQRLPPDAHKPPGGGSFRTPIQEEQADAHSTLAKIYFLG | |
| | RLVPRGRGAAEAATRKQRITETESPYQELQGQRSDVYSDLNTQRPYYK | |
| AA-MSLN-179 | QVQLVQSGGGLVQAGGSLRLSCAPSGSIFGIRTMDWYRQAPGKERELVARITMD | 72 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRFSGSRDGASNAVYLQMNS | |
| | LKPDDTAVYYCRYSGLTSREDYWGPGTQVTVSSFVPVFLPAKPTTTPAPRPPTP | |
| | APTIASQPLSLRPEACRPAAGGAVHTRGLDFACDFWVLVVVGGVLACYSLLVTV | |
| | AFIIFWVRSKRSRLLHSDYMNMTPRRPGPTRKHYQPYAPPRDFAAYRSRLKIQV | |
| | RKAAITSYEKSDGVYTGLSTRNQETYETLKHEKPPQ | |
| AA-MSLN-180 | QVQLVQSGGGLVQAGGSLRLSCAPSGSIFGIRTMDWYRQAPGKERELVARITMD | 73 |
| | GRVFHADSVKGRESGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRFSGSRDGASNAVYLQMNS | |
| | LKPDDTAVYYCRYSGLTSREDYWGPGTQVTVSSFVPVFLPAKPTTTPAPRPPTP | |
| | APTIASQPLSLRPEACRPAAGGAVHTRGLDFACDIYIWAPLAGTCGVLLLSLVI | |
| | TLYCNHRNRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTATKDT | |
| | YDALHMQALPPR | |
| AA-MSLN-181 | QVQLVQSGGGLVQAGGSLRLSCAPSGSIFGIRTMDWYRQAPGKERELVARITMD | 74 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRFSGSRDGASNAVYLQMNS | |
| | LKPDDTAVYYCRYSGLTSREDYWGPGTQVTVSSFVPVFLPAKPTTTPAPRPPTP | |
| | APTIASQPLSLRPEACRPAAGGAVHTRGLDFACDFWVLVVVGGVLACYSLLVTV | |
| | AFIIFWVRSKRSRLLHSDYMNMTPRRPGPTRKHYQPYAPPRDFAAYRSRVKESR | |
| | SADAPAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALPPR | |
| AA-MSLN-182 | QVQLVQSGGGLVQAGGSLRLSCAPSGSIFGIRTMDWYRQAPGKERELVARITMD | 75 |
| | GRVFHADSVKGRFSGSRDGASNAVYLQMNSLKPDDTAVYYCRYSGLTSREDYWG | |
| | PGTQVTVSSGGGGSGGGGSGGGGSQVQLVQSGGGLVQAGGSLRLSCAPSGSIFG | |
| | IRTMDWYRQAPGKERELVARITMDGRVFHADSVKGRFSGSRDGASNAVYLQMNS | |
| | LKPDDTAVYYCRYSGLTSREDYWGPGTQVTVSSFVPVFLPAKPTTTPAPRPPTP | |
| | APTIASQPLSLRPEACRPAAGGAVHTRGLDFACDFWVLVVVGGVLACYSLLVTV | |
| | AFIIFWVRSKRSRLLHSDYMNMTPRRPGPTRKHYQPYAPPRDFAAYRSERVQPL | |
| | EENVGNAARPRFERNKRVKFSRSADAPAYQQGQNQLYNELNLGRREEYDVLDKR | |
| | RGRDPEMGGKPRRKNPQEGLYNELQKDKMAEAYSEIGMKGERRRGKGHDGLYQG | |
| | LSTATKDTYDALHMQALPPR | |
| AA-MSLN-183 | QVQLVQSGGGLVHPGGSLRLSCAASGIDLSLYRMRWYRQAPGKERDLVALITDD | 76 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNRKRGRKKLLYIFKQPFMRPVQTTQEEDGCSCRFPEEEEGGCELRVKES | |
| | RSADAPAYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQALPPR | |
| AA-MSLN-184 | QVQLVQSGGGLVHPGGSLRLSCAASGIDLSLYRMRWYRQAPGKERDLVALITDD | 77 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSESKYGPPCPSCPAPEFEGGPSVF | |
| | LFPPKPKDTLMISRTPEVTCVVVDVSQEDPEVQFNWYVDGVEVHNAKTKPREEQ | |
| | FQSTYRVVSVLTVLHQDWLNGKEYKCKVSNKGLPSSIEKTISKAKGQPREPQVY | |
| | TLPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLGKIYIWAPLAGT | |
| | CGVLLLSLVITLYCNHRNRKRGRKKLLYIFKQPFMRPVQTTQEEDGCSCRFPEE | |
| | EEGGCELRVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLSTATKDTY | |
| | DALHMQALPPR | |
| AA-MSLN-185 | QVQLVQSGGGLVHPGGSLRLSCAASGIDLSLYRMRWYRQAPGKERDLVALITDD | 78 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDFWVLVVVGGVLACYSLLVTVAF | |
| | IIFWVRKRGRKKLLYIFKQPFMRPVQTTQEEDGCSCRFPEEEEGGCELRVKESR | |
| | SADAPAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALPPR | |
| AA-MSLN-186 | QVQLVQSGGGLVHPGGSLRLSCAASGIDLSLYRMRWYRQAPGKERDLVALITDD | 79 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSESKYGPPCPSCPAPEFEGGPSVF | |
| | LFPPKPKDTLMISRTPEVTCVVVDVSQEDPEVQFNWYVDGVEVHNAKTKPREEQ | |
| | FQSTYRVVSVLTVLHQDWLNGKEYKCKVSNKGLPSSIEKTISKAKGQPREPQVY | |
| | TLPPSQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLGKFWVLVVVGGV | |
| | LACYSLLVTVAFIIFWVRKRGRKKLLYIFKQPFMRPVQTTQEEDGCSCRFPEEE | |
| | EGGCELRVKFSRSADAPAYQQGQNQLYNELNLGRREEYDVLDKRRGRDPEMGGK | |
| | PRRKNPQEGLYNELQKDKMAEAYSEIGMKGERRRGKGHDGLYQGLSTATKDTYD | |
| | ALHMQALPPR | |
| AA-MSLN-187 | QVQLVQSGGGLVHPGGSLRLSCAASGIDLSLYRMRWYRQAPGKERDLVALITDD | 80 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNWRRKRKEKQSETSPKEFLTIYEDVKDLKTRRNHEQEQTFPGGGSTIYS | |
| | MIQSQSSAPTSQEPAYTLYSLIQPSRKSGSRKRNHSPSENSTIYEVIGKSQPKA | |
| | QNPARLSRKELENFDVYSRVKFSRSADAPAYQQGQNQLYNELNLGRREEYDVLD | |
| | KRRGRDPEMGGKPRRKNPQEGLYNELQKDKMAEAYSEIGMKGERRRGKGHDGLY | |
| | QGLSTATKDTYDALHMQALPPR | |
| AA-MSLN-188 | QVQLVQSGGGLVHPGGSLRLSCAASGIDLSLYRMRWYRQAPGKERDLVALITDD | 81 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNLCARPRRSPAQEDGKVYINMPGRGRVKFSRSADAPAYQQGQNQLYNEL | |
| | NLGRREEYDVLDKRRGRDPEMGGKPRRKNPQEGLYNELQKDKMAEAYSEIGMKG | |
| | ERRRGKGHDGLYQGLSTATKDTYDALHMQALPPR | |
| AA-MSLN-189 | QVQLVQSGGGLVHPGGSLRLSCAASGIDLSLYRMRWYRQAPGKERDLVALITDD | 82 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVELQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNYFLGRLVPRGRGAAEAATRKQRITETESPYQELQGQRSDVYSDLNTQR | |
| | PYYKRVKFSRSADAPAYQQGQNQLYNELNLGRREEYDVLDKRRGRDPEMGGKPR | |
| | RKNPQEGLYNELQKDKMAEAYSEIGMKGERRRGKGHDGLYQGLSTATKDTYDAL | |
| | HMQALPPR | |
| AA-MSLN-190 | QVQLVQSGGGLVHPGGSLRLSCAASGIDLSLYRMRWYRQAPGKERDLVALITDD | 83 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNALYLLRRDQRLPPDAHKPPGGGSFRTPIQEEQADAHSTLAKIRVKFSR | |
| | SADAPAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALPPR | |
| AA-MSLN-191 | QVQLVQSGGGLVHPGGSLRLSCAASGIDLSLYRMRWYRQAPGKERDLVALITDD | 84 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDFWVLVVVGGVLACYSLLVTVAF | |
| | IIFWVRSKRSRLLHSDYMNMTPRRPGPTRKHYQPYAPPRDFAAYRSYFLGRLVP | |
| | RGRGAAEAATRKQRITETESPYQELQGQRSDVYSDLNTQRPYYK | |
| AA-MSLN-192 | QVQLVQSGGGLVHPGGSLRLSCAASGIDLSLYRMRWYRQAPGKERDLVALITDD | 85 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNRKRGRKKLLYIFKQPFMRPVQTTQEEDGCSCRFPEEEEGGCELYFLGR | |
| | LVPRGRGAAEAATRKQRITETESPYQELQGQRSDVYSDLNTQRPYYK | |
| AA-MSLN-193 | QVQLVQSGGGLVHPGGSLRLSCAASGIDLSLYRMRWYRQAPGKERDLVALITDD | 86 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNALYLLRRDQRLPPDAHKPPGGGSFRTPIQEEQADAHSTLAKIYFLGRL | |
| | VPRGRGAAEAATRKQRITETESPYQELQGQRSDVYSDLNTQRPYYK | |
| AA-MSLN-194 | QVQLVQSGGGLVHPGGSLRLSCAASGIDLSLYRMRWYRQAPGKERDLVALITDD | 87 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDFWVLVVVGGVLACYSLLVTVAF | |
| | IIFWVRSKRSRLLHSDYMNMTPRRPGPTRKHYQPYAPPRDFAAYRSRLKIQVRK | |
| | AAITSYEKSDGVYTGLSTRNQETYETLKHEKPPQ | |
| AA-MSLN-195 | QVQLVQSGGGLVHPGGSLRLSCAASGIDLSLYRMRWYRQAPGKERDLVALITDD | 88 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDIYIWAPLAGTCGVLLLSLVITL | |
| | YCNHRNRVKFSRSADAPAYQQGQNQLYNELNLGRREEYDVLDKRRGRDPEMGGK | |
| | PRRKNPQEGLYNELQKDKMAEAYSEIGMKGERRRGKGHDGLYQGLSTATKDTYD | |
| | ALHMQALPPR | |
| AA-MSLN-196 | QVQLVQSGGGLVHPGGSLRLSCAASGIDLSLYRMRWYRQAPGKERDLVALITDD | 89 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDFWVLVVVGGVLACYSLLVTVAF | |
| | IIFWVRSKRSRLLHSDYMNMTPRRPGPTRKHYQPYAPPRDFAAYRSRVKESRSA | |
| | DAPAYQQGQNQLYNELNLGRREEYDVLDKRRGRDPEMGGKPRRKNPQEGLYNEL | |
| | QKDKMAEAYSEIGMKGERRRGKGHDGLYQGLSTATKDTYDALHMQALPPR | |
| AA-MSLN-197 | QVQLVQSGGGLVHPGGSLRLSCAASGIDLSLYRMRWYRQAPGKERDLVALITDD | 90 |
| | GTSYYEDSVKGRFTITRDNPSNKVFLQMNSLKPEDTAVYYCNAETPLSPVNYWG | |
| | QGTQVTVSGGGGSGGGGSGGGGSQVQLVQSGGGLVHPGGSLRLSCAASGIDLSL | |
| | YRMRWYRQAPGKERDLVALITDDGTSYYEDSVKGRFTITRDNPSNKVFLQMNSL | |
| | KPEDTAVYYCNAETPLSPVNYWGQGTQVTVSFVPVFLPAKPTTTPAPRPPTPAP | |
| | TIASQPLSLRPEACRPAAGGAVHTRGLDFACDFWVLVVVGGVLACYSLLVTVAF | |
| | IIFWVRSKRSRLLHSDYMNMTPRRPGPTRKHYQPYAPPRDFAAYRSERVQPLEE | |
| | NVGNAARPRFERNKRVKFSRSADAPAYQQGQNQLYNELNLGRREEYDVLDKRRG | |
| | RDPEMGGKPRRKNPQEGLYNELQKDKMAEAYSEIGMKGERRRGKGHDGLYQGLS | |
| | TATKDTYDALHMQALPPR | |

In some embodiments, the antigen binding domain of a CAR described herein specifically binds to mesothelin. In some embodiments, the antigen binding domain of the CAR is derived from and/or comprises a camelid VHH. In some embodiments, the antigen binding domain of the CAR is derived from and/or comprises a humanized camelid VHH. In some embodiments, the antigen binding domain of the CAR comprises one or more amino acid substitutions (e.g., framework amino acid substitutions). In some embodiments, the antigen binding domain of the CAR comprises two or three VHH amino acid sequences. In some embodiments, the antigen binding domain of the CAR comprises two copies of the same VHH amino acid sequence. In some embodiments, the antigen binding domain of the CAR comprises one copy of a first VHH amino acid sequence and one copy of a second VHH amino acid sequence (e.g., the antigen binding domain of the CAR may include two or three copies of SEQ ID NO: 97 (e.g., as described or including one or more of the specific amino acid substitutions described below), two or three copies of SEQ ID NO: 98, one copy of SEQ ID NO: 97 and one copy of SEQ ID NO: 98 (e.g., as described or including one or more of the specific amino acid substitutions described below), one copy of SEQ ID NO: 97 and two copies of SEQ ID NO: 98, two copies of SEQ ID NO: 97 and one copy of SEQ ID NO: 98). In some embodiments, when the antigen binding domain of a CAR comprises two or three VHHs amino acid sequences, said antigen binding domain includes a linker disposed between the VHH amino acid sequences. Any suitable linker may be disposed between the VHH amino acid sequences of the CAR (e.g., (Gly₄-Ser)ₙ, wherein n is a positive integer equal to or greater than 1 (e.g., n may be equal to 1, 2, 3, 4, 5, 6, 7, 8, 9, or 10) (SEQ ID NO: 130) or (Gly₄-Ser)ₙ, wherein n is a positive integer equal to or greater than 1 (e.g., n may be equal to 1, 2, 3, 4, 5, 6, 7, 8, 9, or 10) (SEQ ID NO: 131). In some embodiments, the linker comprises the amino acid sequence (Gly₄Ser)₄ (SEQ ID NO: 132) or (Gly₄Ser)₃ (SEQ ID NO: 133). In some embodiments, the linker comprises multiple repeats (e.g., 2, 3, 4, 5, 6, 7, 8, 9, or 10) of (GlySer), (Gly₂Ser), or (Gly₃Ser).

In some embodiments, the antigen binding domain of the CAR comprises one or more (e.g., one, two or all three) of a complementarity determining region 1 (CDR1) comprising or consisting of GIDLSLYR (SEQ ID NO: 91), a complementarity determining region 2 (CDR2) comprising or consisting of ITDDGTS (SEQ ID NO: 92) and a complementarity determining region 3 (CDR3) comprising or consisting of NAETPLSPVNY (SEQ ID NO: 93). In some embodiments, the antigen binding domain of the CAR comprises one or more (e.g., one, two or all three) of a CDR1 comprising or consisting of GSIFGIRT (SEQ ID NO: 94), a CDR2 comprising or consisting of ITMDGRV (SEQ ID NO: 95) and a CDR3 comprising or consisting of RYSGLTSREDY (SEQ ID NO: 96).

In some embodiments, the antigen binding domain of a CAR described herein comprises an amino acid sequence that is at least 80%, at least 85%, at least 90%, at least 95%, at least 96%, at least 97%, at least 98%, at least 99%, or 100% identical to any one of SEQ ID NOs: 97, 98, 99, 100, 101, and 102 (provided below).

MLSN Binder-001
(SEQ ID NO: 97)
QVQLVQSGGGLVHPGGSLRLSCAASGIDLSLYRMRWYRQAPGKERDLVA
LITDDGTSYYEDSVKGRFTITRDNPSNKVFLQMNSLKPEDTAVYYCNAE
TPLSPVNYWGQGTQVTVS
MLSN Binder-002
(SEQ ID NO: 98)
QVQLVQSGGGLVQAGGSLRLSCAPSGSIFGIRTMDWYRQAPGKERELVA
RITMDGRVFHADSVKGRESGSRDGASNAVYLQMNSLKPDDTAVYYCRYS
GLTSREDYWGPGTQVTVSS
MLSN Binder-003
(SEQ ID NO: 99)
QVQLVQSGGGLVHPGGSLRLSCAASGIDLSLYRMRWYRQAPGKERDLVA
LITDDGTSYYEDSVKGRFTITRDNPSNKVFLQMNSLKPEDTAVYYCNAE
TPLSPVNYWGQGTQVTVSGGGGSGGGGSGGGGSQVQLVQSGGGLVQAGG
SLRLSCAPSGSIFGIRTMDWYRQAPGKERELVARITMDGRVFHADSVKG
RFSGSRDGASNAVYLQMNSLKPDDTAVYYCRYSGLTSREDYWGPGTQVT
VSS
MLSN Binder-004
(SEQ ID NO: 100)
QVQLVQSGGGLVHPGGSLRLSCAASGIDLSLYRMRWYRQAPGKERDLVA
LITDDGTSYYEDSVKGRFTITRDNPSNKVFLQMNSLKPEDTAVYYCNAE
TPLSPVNYWGQGTQVTVSGGGGSGGGGSGGGGSQVQLVQSGGGLVHPGG
SLRLSCAASGIDLSLYRMRWYRQAPGKERDLVALITDDGTSYYEDSVKG
RFTITRDNPSNKVFLQMNSLKPEDTAVYYCNAETPLSPVNYWGQGTQVT
VS
MLSN Binder-005
(SEQ ID NO: 101)
QVQLVQSGGGLVQAGGSLRLSCAPSGSIFGIRTMDWYRQAPGKERELVA
RITMDGRVFHADSVKGRFSGSRDGASNAVYLQMNSLKPDDTAVYYCRYS
GLTSREDYWGPGTQVTVSSGGGGSGGGGSGGGGSQVQLVQSGGGLVQAG
GSLRLSCAPSGSIFGIRTMDWYRQAPGKERELVARITMDGRVFHADSVK
GRESGSRDGASNAVYLQMNSLKPDDTAVYYCRYSGLTSREDYWGPGTQV
TVSS
MLSN Binder-006
(SEQ ID NO: 102)
QVQLVQSGGGLVHPGGSLRLSCAASGIDLSLYRMRWYRQAPGKERDLVA
LITDDGTSYYEDSVKGRFTITRDNPSNKVFLQMNSLKPEDTAVYYCNAE
TPLSPVNYWGQGTQVTVSGGGGSGGGGSGGGGSQVQLVQSGGGLVHPGG
SLRLSCAASGIDLSLYRMRWYRQAPGKERDLVALITDDGTSYYEDSVKG
RFTITRDNPSNKVFLQMNSLKPEDTAVYYCNAETPLSPVNYWGQGTQVT
VS

In some embodiments, the antigen binding domain of the CAR comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 97, and has one or more amino acid substitutions (e.g., framework amino acid substitutions) as compared to the amino acid sequence of SEQ ID NO: 97. In some embodiments, the antigen binding domain of the CAR comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 97 and has one or more (e.g., one, two, three, four, five, six, seven, eight, nine, ten, eleven, twelve, thirteen, fourteen, fifteen, or more) of the following amino acid substitutions as compared to SEQ ID NO: 97: Q6E, H13Q, M34L, R35G, K43Q, E44G, R45L, D46E, D46A, L47G, L47A, E60A, T70S, P74S, S75K, K77T, V78L, F79Y, K86R, P87A, and Q112L. In some embodiments, the antigen binding domain of the CAR comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to SEQ ID NO: 97 and has one or both of the following amino acid substitutions as compared to the amino acid sequence of SEQ ID NO: 97: E44G and R45L. In some embodiments, the antigen binding domain of the CAR comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99/a, or 100% identical to QVQLVESGGGLVQPGGSLRLSCAASGIDLSLYRLGWYRQAPGQGLDLVALITDDGTSY YADSVKGRFTISRDNSKNTLYLQMNSLRAEDTAVYYCNAETPLSPVNYWGQGTLVTVS (SEQ ID NO: 103). In some embodiments, the antigen binding domain of the CAR comprises two or three copies of an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 97, and has one or more amino acid substitutions (e.g., framework amino acid substitutions) as compared to the amino acid sequence of SEQ ID NO: 97. In some embodiments, the antigen binding domain of the CAR comprises two or three copies of an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 97, and has one or more (e.g., one, two, three, four, five, six, seven, eight, nine, ten, eleven, twelve, thirteen, fourteen, fifteen, or more) of the following amino acid substitutions as compared to the amino acid sequence of SEQ ID NO: 97: Q6E, H13Q, M34L, R35G, K43Q, E44G, R45L, D46E, D46A, L47G, L47A, E60A, T70S, P74S, S75K, K77T, V78L, F79Y, K86R, P87A, and Q 112L. In some embodiments, the antigen binding domain of the CAR comprises two or three copies of an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to SEQ ID NOs: 97 and includes one or both of the following amino acid substitutions as compared to the amino acid sequence of SEQ ID NO: 97: E44G and R45L. In some embodiments, the antigen binding domain of the CAR comprises two or three copies of an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 103.

In some embodiments, the antigen binding domain of the CAR comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 98, and has one or more amino acid substitutions (e.g., framework amino acid substitutions) as compared to the amino acid sequence of SEQ ID NO: 98. In some embodiments, the antigen binding domain of the CAR comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 97 and has one or more (e.g., one, two, three, four, five, six, seven, eight, nine, ten, eleven, twelve, thirteen, fourteen, fifteen, or more) of the following amino acid substitutions as compared to the amino acid sequence of SEQ ID NO: 98: Q6E, A14P, P24A, M34L, D35G, K43Q, E44G, R45L, L47G, L47A, F58Y, H59Y, S68T, G69I, G73N, A74S, S75K, A77T, V78L, K86R, P87A, D88E, P109Q, and Q112L. In some embodiments, the antigen binding domain of the CAR comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to SEQ ID NOs: 98, and has one or both of the following amino acid substitutions as compared to the amino acid sequence of SEQ ID NO: 98: E44G and R45L. In some embodiments, the antigen binding domain of the CAR comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to QVQLVESGGGLVQPGGSLRLSCAASGSIFGIRTLGWYRQ APGQGLELVARITMDGR VYYADSVKGRFTISRDNSKNTLYL (SEQ ID NO: 104). In some embodiments, the antigen binding domain of the CAR comprises two or three copies of an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 98, and has one or more amino acid substitutions (e.g., framework amino acid substitutions) as compared to the amino acid sequence of SEQ ID NO: 98. In some embodiments, the antigen binding domain of the CAR comprises two or three copies of an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 97 and has one or more (e.g., one, two, three, four, five, six, seven, eight, nine, ten, eleven, twelve, thirteen, fourteen, fifteen, or more) of the following amino acid substitutions as compared to the amino acid sequence of SEQ ID NO: 98: Q6E, A14P, P24A, M34L, D35G, K43Q, E44G, R45L, L47G, L47A, F58Y, H59Y, S68T, G69I, G73N, A74S, S75K, A77T, V78L, K86R, P87A, D88E, P109Q, and Q112L. In some embodiments, the antigen binding domain of the CAR comprises two or three copies of an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to SEQ ID NOs: 98, and have one or both of the following amino acid substitutions as compared to the amino acid sequence of SEQ ID NO: 98: E44G and R45L. In some embodiments, the antigen binding domain of the CAR comprises two or three copies an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to QVQLVESGGGLVQPGGSLRLSCAASGSIFGIRTLGWYRQ APGQGLELVARITMDGR VYYADSVKGRFTISRDNSKNTLYL (SEQ ID NO: 104).

### Hinge Domain

In some embodiments, a CAR provided herein includes hinge domain (also known as a spacer domain) between the antigen binding domain and the transmembrane domain of the CAR. In some embodiments, the hinge domain provides flexibility for antigen binding domain to bind the target antigen (e.g., mesothelin). In some embodiments, the hinge domain is up to about 300 amino acids in length. In some embodiments, the hinge domain is from about 10 to about 100 amino acids in length. In some embodiments, the hinge domain is from about 25 to about 50 amino acids in length. In some embodiments, the hinge domain comprises or consists of a CD8a (e.g., a human CD8a hinge domain), IgG1 (e.g., an IgG1 hinge domain or IgG1 short hinge domain), IgG4 (e.g., an IgG4 hinge domain, an IgG4 short hinge domain, an IgG4 hinge-CH3, IgG4 mutant hinge domain, an IgG4 mutant-1 hinge domain, or an IgG4 mutant-2 hinge domain) or CD28 hinge domain.

Hinge domains may be derived from CD8, CD8α, CD4, CD28, 4-1BB, or IgG (in particular, the hinge domain of an IgG, for example from IgG1, IgG2, IgG3, or IgG4), and from an antibody heavy-chain constant region. Alternatively, the hinge domain may be a synthetic sequence. In some embodiments, the hinge domain comprises all or part of the hinge region of an immunoglobulin (e.g., human IgG1, IgG2, IgG3, or IgG4). In some embodiments, the hinge domain comprises an immunoglobulin CH2 and/or CH3 domain. In some embodiments, the hinge domain comprises a CH3 region of a human immunoglobulin. In some embodiments, the hinge domain is from an IgG (e.g., IgG1, IgG2, IgG3 or IgG4) and the domain comprises one or more mutations (e.g., amino acid substitutions (e.g., in its CH2 domain) so as to prevent or reduce off-target binding of the hinge domain and/or a CAR comprising the hinge domain to an Fc receptor. In some embodiments, the hinge domain is derived from an IgG1, IgG2, IgG3, or IgG4 Fc region and includes one or more amino acid substitutions as compared to the wild-type protein from which the hinge domain was derived. In some embodiments, the hinge domain is derived from an IgG1, IgG2, IgG3, or IgG4 Fc region and includes one or more (e.g., two, three, four, five, six, seven, eight, nine, ten, eleven, twelve, thirteen, fourteen, fifteen, twenty, twenty-five, thirty, or more) amino acid substitutions at an amino acid residue at position 220, 226, 228, 229, 230, 233, 234, 235, 234, 237, 238, 239, 243, 247, 267, 268, 280, 290, 292, 297, 298, 299, 300, 305, 309, 318, 326, 330, 331, 332, 333, 334, 336, and/or 339 (amino acid residue positions indicated in the EU index proposed in Kabat et al. (1991) Sequences of Proteins of Immunological Interest, 5th Ed., United States Public Health Service, National Institutes of Health, Bethesda). In some embodiments, the hinge domain is derived from an IgG1, IgG2, IgG3, or IgG4 Fc region and includes one or more (e.g., two, three, four, five, six, seven, eight, nine, ten, eleven, twelve, thirteen, fourteen, fifteen, twenty, twenty-five, thirty, or more) of the following amino acid substitutions C220S, C226S, S228P, C229S, P230S, E233P, V234A, L234V, L234F, L234A, L235A, L235E, G236A, G237A, P238S, S239D, F243L, P247I, S267E, H268Q, S280H, K290S, K290E, K290N, R292P, N297A, N297Q, S298A, S298G, S298D, S298V, T299A, Y300L, V305I, V309L, E318A, K326A, K326W, K326E, L328F, A330L, A330S, A33IS, P331S, I332E, E333A, E333S, E333S, K334A, A339D, A339Q, and P396L. In some embodiments, the hinge domain is derived from an IgG1, IgG2, IgG3, or IgG4 Fc region and includes one or more of the following combinations of amino acid substitutions: S228P and L235E; S228P and N297Q; L235E and N297Q; S228P, L235E, and N297Q.

Examples of hinge domains are provided in Table 2 below. In some embodiments, the CAR comprises a hinge domain comprising an amino acid sequence having at least 90%, 91%, 92%, 93%, 94%, 95%, 96%, 97%, 98%, 99% or 100% identity to any one of SEQ ID NOs: 105-117. In some embodiments, the CAR comprises a hinge domain comprising or consisting of the amino acid sequence of any one of SEQ ID NOs: 105-117.

TABLE 2
Examples of Hinge Domains

| | | SEQ ID |
|---|---|---|
| Name | Amino Acid Sequence | NO: |
| human CD8α hinge domain | TTTPAPRPPTPAPTIASQPLSLRPEACRPAAGGAVHTRGLDFACD | 105 |
| human CD8α hinge domain | FVPVFLPAKPTTTPAPRPPTPAPTIASQPLSLRPEACRPAAGGAVH | 106 |
| | TRGLDFACD | |
| human IgG1 hinge domain | EPKSCDKTHTCPPCPAPELLGGPSVFLFPPKPKDTLMISRTPEVTC | 107 |
| | VVVDVSHEDPEVKFNWYVDGVEVHNAKTKPREEQYNSTYRVVSVLT | |
| | VLHQDWLNGKEYKCKVSNKALPAPIEKTISKAKGQPREPQVYTLPP | |
| | SRDELTKNQVSLTCLVKGFYPSDIAVEWESNGQPENNYKTTPPVLD | |
| | SDGSFFLYSKLTVDKSRWQQGNVFSCSVMHEALHNHYTQKSLSLSP | |
| | GK | |
| human IgG1 hinge domain | EPKSCDKTHTCPPCPAPELLGGPSVFLFPPKPKDTLMISRTPEVTC | 108 |
| | VVVDVSHEDPEVKFNWYVDGVEVHNAKTKPREEQYNSTYRVVSVLT | |
| | VLHQDWLNGKEYKCKVSNKALPAPIEKTISKAKGQPREPQVYTLPP | |
| | SRDELTKNQVSLTCLVKGFYPSDIAVEWESNGQPENNYKTTPPVLD | |
| | SDGSFFLYSKLTVDKSRWQQGNVFSCSVMHEALHNHYTQKSLSLSP | |
| | GK | |
| human IgG4 hinge domain | ESKYGPPCPSCPAPEFLGGPSVFLFPPKPKDTLMISRTPEVTCVVV | 109 |
| | DVSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| human FcγRIIIα hinge | GLAVSTISSFFPPGYQ | 110 |
| domain | | |
| CD28 hinge domain | IEVMYPPPYLDNEKSNGTIIHVKGKHLCPSPLFPGPSKP | 111 |
| IgG1 short hinge domain | AEPKSPDKTHTCPPCPKDP | 112 |
| IgG4 short hinge domain | ESKYGPPCPSCP | 113 |
| IgG4 hinge-CH3 hinge | ESKYGPPCPSCPGQPREPQVYTLPPSQEEMTKNQVSLTCLVKGFYP | 114 |
| domain | SDIAVEWESNGQPENNYKTTPPVLDSDGSFFLYSRLTVDKSRWQEG | |
| | NVFSCSVMHEALHNHYTQKSLSLSLGK | |
| IgG4 mutant hinge domain | ESKYGPPCPSCPAPEFEGGPSVFLFPPKPKDTLMISRTPEVTCVVV | 115 |
| | DVSQEDPEVQFNWYVDGVEVHNAKTKPREEQFQSTYRVVSVLTVLH | |
| | QDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| IgG4 mutant-1 hinge | ESKYGPPCPSCPAPEFEGGPSVFLFPPKPKDTLMISRTPEVTCVVV | 116 |
| domain | DVSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| IgG4 mutant-2 hinge | ESKYGPPCPSCPAPEFLGGPSVFLFPPKPKDTLMISRTPEVTCVVV | 117 |
| domain | DVSQEDPEVQFNWYVDGVEVHNAKTKPREEQFQSTYRVVSVLTVLH | |
| | QDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |

### Transmembrane Domain

In some embodiments, the CARs described herein include a transmembrane domain attached to the extracellular portion of the CAR. A transmembrane domain can include one or more additional amino acid residues adjacent to the transmembrane region (e.g., one or more (e.g., 1, 2, 3, 4, 5, 6, 7, 8, 9, 10, 11, 12, 13, 14, or 15) amino acid residues associated with the extracellular region of the protein from which the transmembrane domain was derived and/or one or more (e.g., 1, 2, 3, 4, 5, 6, 7, 8, 9, 10, 11, 12, 13, 14, or 15) amino acid residues associated with the intracellular region of the protein from which the transmembrane domain was derived). In some embodiments, the transmembrane domain may be derived from the same protein that a signaling domain of the CAR is derived. In some embodiments, the transmembrane domain is not derived from the same protein that a signaling domain of the CAR is derived. The transmembrane domain can be derived either from a natural or synthetic source. The transmembrane domain can be derived from any membrane-bound or transmembrane protein. For example, the transmembrane domain can be a human CD8a transmembrane domain, a CD8a transmembrane domain, a CD28 transmembrane domain, a NKG2D transmembrane domain, a NKG2D transmembrane domain, a CD16 transmembrane domain, a NKp44 transmembrane domain, a NKp46 transmembrane domain, a CD27 transmembrane domain, a CD27 transmembrane domain, a DAP12 transmembrane domain, or a DAP10 transmembrane domain. Examples of transmembrane domains are provided in Table 3. In some embodiments, the CAR comprises a transmembrane domain comprising an amino acid sequence having at least 90%, 91%, 92%, 93%, 94%, 95%, 96%, 97%, 98%, 99% or 100% identity to any one of SEQ ID NOs: 118-129. In some embodiments, the CAR comprises a transmembrane domain comprising or consisting of the amino acid sequence of any one of SEQ ID NOs: 118-129.

TABLE 3
Examples of Transmembrane Domains

| Name | Amino Acid Sequence | SEQ ID NO: |
|---|---|---|
| human CD8α transmembrane domain | IYIWAPLAGTCGVLLLSLVIT | 118 |
| human CD8α transmembrane domain | IYIWAPLAGTCGVLLLSLVITLYCNHR | 119 |
| | N | |
| human CD28 transmembrane domain | FWVLVVVGGVLACYSLLVTVAFIIFWV | 120 |
| human NKG2D transmembrane domain | VVRVLAIALAIRFTLNTLMWLAI | 121 |
| human NKG2D transmembrane domain | PFFFCCFIAVAMGIRFIIMVAIWSAVF | 122 |
| | LNS | |
| human CD16 transmembrane domain | VSFCLVMVLLFAVDTGLYFSV | 123 |
| human NKp44 transmembrane domain | LVPVFCGLLVAKSLVLSALLV | 124 |
| human NKp46 transmembrane domain | MGLAFLVLVALVWFLVEDWLS | 125 |
| human CD27 transmembrane domain | ILVIFSGMFLVFTLAGALFL | 126 |
| human CD27 transmembrane domain | ILVIFSGMFLVFTLAGALFLH | 127 |
| human DAP12 transmembrane domain | GVLAGIVMGDLVLTVLIALAV | 128 |
| human DAP10 transmembrane domain | LLAGLVAADAVASLLIVGAVE | 129 |

Optionally, a CAR provided herein may include a short polypeptide linker, in some embodiments, between 2 and 10 amino acids in length may form the linkage between the transmembrane domain and the cytoplasmic signaling domain of the CAR. In some embodiments, the linker is a glycine-serine linker. In some embodiments, the linker comprises either the amino acid sequence (Gly₃-Ser)ₙ, wherein n is a positive integer equal to or greater than 1 (e.g., n may be equal to 1, 2, 3, 4, 5, 6, 7, 8, 9, or 10) (SEQ ID NO: 130) or (Gly₄-Ser)ₙ, wherein n is a positive integer equal to or greater than 1 (e.g., n may be equal to 1, 2, 3, 4, 5, 6, 7, 8, 9, or 10) (SEQ ID NO: 131). In some embodiments, the linker comprises the amino acid sequence (Gly₄Ser)₄ (SEQ ID NO: 132) or (Gly₄Ser)₃ (SEQ ID NO: 133). In some embodiments, the linker comprises multiple repeats (e.g., 2, 3, 4, 5, 6, 7, 8, 9, or 10) of (GlySer), (Gly₂Ser), or (Gly₃Ser).

### Signaling Domains

The cytoplasmic domain or region of a CAR provided herein includes at least one signaling domain. In some embodiments, the signaling domain of the CAR mediates activation of at least one of the effector functions of the immune cell (e.g., an NK cell) in which the CAR is present. Effector functions include cytolytic activity or helper activity, including the secretion of cytokines. In some embodiments, the signaling domain comprises one or more (e.g., one, two, three, four, or more) immunoreceptor tyrosine-based activation motifs (ITAMs). In some embodiments, a CAR provided herein includes one or more (e.g., one, two, three, or more) signaling domains. When the CAR includes one or more signaling domains, the signaling domains may be linked to each other in a random or specified order. Optionally, a linker may be disposed between each signaling domain. In some embodiments, the linker is from between about 1 to about 10 (e.g., 1, 2, 3, 4, 5, 6, 7, 8, 9 or 10) amino acid residues in length. In some embodiments, the linker is a glycine-serine doublet. In some embodiments, the linker is a single amino acid residue (e.g., an alanine or glycine residue). The CAR may include one or more signaling domains selected from human CD28 signaling domain, CD28 signaling domain, 4-1BB signaling domain, DAP10 signaling domain, DAP12 signaling domain, 2B4 signaling domain, OX40 signaling domain, CD27 signaling domain, CD27 signaling domain, OX40L signaling domain, CD3zeta signaling domain, FCER1G signaling domain, and FCGR2A signaling domain. Examples of signaling domains are provided in Table 4 below. In some embodiments, the CAR comprises one or more (e.g., 2, 3, 4, or 4) signaling domains each comprising an amino acid sequence having at least 90%, 91%, 92%, 93%, 94%, 95%, 96%, 97%, 98%, 99% or 100% identity to any one of SEQ ID NOs: 134-149. In some embodiments, the CAR comprises one or more (e.g., 2, 3, 4, or 5) signaling domains each comprising or consisting of the amino acid sequence of any one of SEQ ID NOs: 134-149.

TABLE 4
Examples of Signaling Domains

| | | SEQ ID |
|---|---|---|
| Name | Amino Acid Sequence | NO: |
| human CD28 signaling domain | RSKRSRGGHSDYMNMTPRRPGPTRKHYQPYAPPRDFAAYRS | 134 |
| human CD28 signaling domain | RSKRSRLLHSDYMNMTPRRPGPTRKHYQPYAPPRDFAAYRS | 135 |
| human 4-1BB signaling domain | KRGRKKLLYIFKQPFMRPVQTTQEEDGCSCRFPEEEEGGCE | 136 |
| | I | |
| human 4-1BB signaling domain | RKRGRKKLLYIFKQPFMRPVQTTQEEDGCSCRFPEEEEGGC | 137 |
| | EL | |
| human DAP10 signaling domain | LCARPRRSPAQEDGKVYINMPGRG | 138 |
| human DAP10 signaling domain | CARPRRSPAQEDGKVYINMPGRG | 139 |
| human DAP12 signaling domain | YFLGRLVPRGRGAAEAATRKQRITETESPYQELQGQRSDVY | 140 |
| | SDLNTQRPYYK | |
| human 2B4 signaling domain | WRRKRKEKQSETSPKEFLTIYEDVKDLKTRRNHEQEQTFPG | 141 |
| | GGSTIYSMIQSQSSAPTSQEPAYTLYSLIQPSRKSGSRKRN | |
| | HSPSENSTIYEVIGKSQPKAQNPARLSRKELENFDVYS | |
| human OX40 signaling domain | ALYLLRRDQRLPPDAHKPPGGGSFRTPIQEEQADAHSTLAK | 142 |
| | I | |
| human CD27 costimulatory | HQRRKYRSNKGESPVEPAEPCHYSCPREEEGSTIPIQEDYR | 143 |
| domain | KPEPACSP | |
| human CD27 costimulatory | QRRKYRSNKGESPVEPAEPCHYSCPREEEGSTIPIQEDYRK | 144 |
| domain | PEPACSP | |
| human OX40L costimulatory | ERVQPLEENVGNAARPRFERNK | 145 |
| domain | | |
| human CD3zeta intracellular | RVKFSRSADAPAYKQGQNQLYNELNLGRREEYDVLDKRRGR | 146 |
| signaling domain | DPEMGGKPRRKNPQEGLYNELQKDKMAEAYSEIGMKGERRR | |
| | GKGHDGLYQGLSTATKDTYDALHMQALPPR | |
| human CD3zeta intracellular | RVKFSRSADAPAYQQGQNQLYNELNLGRREEYDVLDKRRGR | 147 |
| signaling domain | DPEMGGKPRRKNPQEGLYNELQKDKMAEAYSEIGMKGERRR | |
| | GKGHDGLYQGLSTATKDTYDALHMQALPPR | |
| human FCER1G intracellular | RLKIQVRKAAITSYEKSDGVYTGLSTRNQETYETLKHEKPP | 148 |
| signaling domain | Q | |
| human FCGR2A intracellular | CRKKRISANSTDPVKAAQFEPPGRQMIAIRKRQLEETNNDY | 149 |
| signaling domain | ETADGGYMTLNPRAPTDDDKNIYLTLPPNDHVNSNN | |

### Signal Peptides

In some embodiments, any one of the CARs described herein comprises at their N-terminus a signal peptide (e.g., disposed N-terminal to the antigen binding domain of the CAR). Signal peptides may be cleaved from the antigen binding domain during cellular processing and localization of the CAR to the cellular membrane of an immune cell. Examples of signal peptides include human CD8a signal peptide (MALPVTALLLPLALLLHAARP; SEQ ID NO: 150), human CD27 signal peptide (MARPHPWWLCVLGTLVGLS; SEQ ID NO: 151) and human IgG signal peptide (MEFGLSWLFLVAILKGVQCSR; SEQ ID NO: 152). In some embodiments, any one of the CARs provided herein do not comprise a signal peptide.

### Exogenous Polypeptides

The present disclosure provides a population of immune cells (e.g., T cells or NK cells) engineered to express a CAR (e.g., any of the exemplary CARs described herein) and one or more (e.g., one, two, three or more) exogenous polypeptides described herein. In some embodiments, the exogenous polypeptide is capable of being secreted by the immune cell. In some embodiments, the exogenous polypeptide is membrane bound (e.g., to the plasma membrane of the immune cell).

In some embodiments, the exogenous polypeptide comprises interleukin-15 (IL-15) or a functional fragment thereof. In some embodiments, the IL-15 or the functional fragment thereof comprises an amino acid sequence having at least 90%, 91%, 92%, 93%, 94%, 95%, 96%, 97%, 98%, 99%, or 100% identity to MRISKPHLRSISIQCYLCLLLNSHFLTEAGIHVFILGCFSAGLPKTEANWVNVISDLKKIEDLIQSMHIDATLYTES DVHPSCKVTAMKCFLLELQVISLESGDASIHDTVENLIILANNSLSSNGNVTESGCKECEELEEKNIKEFLQSFVHI VQMFINTS (SEQ ID NO: 153). In some embodiments, the IL-15 or the functional fragment thereof comprises an amino acid sequence having at least 90%, 91%, 92%, 93%, 94%, 95%, 96%, 97%, 98%, 99%, or 100% identity to NWVNVISDLKKIEDLIQSMHIDATLYTESDVHPSCKVTAMKCFLLELQVISLESGDASIHDTVENLIILANNSLSSN GNVTESGCKECEELEEKNIKEFLQSFVHIVQMFINTS (SEQ ID NO: 154).

In some embodiments, the exogenous polypeptide comprises IL-15 receptor alpha (IL-15Ra) or a functional fragment thereof. In some embodiments, the IL-15Ra or the functional fragment thereof comprises an amino acid sequence having at least 90%, 91%, 92%, 93%, 94%, 95%, 96%, 97%, 98%, 99% or 100% identity to MAPRRARGCRTLGLPALLLLLLLRPPATRGITCPPPMSVEHADIWVKSYSLYSRERYICNSGFKRKAGTSSLTECVL NKATNVAHWTTPSLKCIRDPALVHQRPAPPSTVTTAGVTPQPESLSPSGKEPAASSPSSNNTAATTAAIVPGSQLMP SKSPSTGTTEISSHESSHGTPSQTTAKNWELTASASHQPPGVYPQGHSDTTVAISTSTVLLCGLSAVSLLACYLKSR QTPPLASVEMEAMEALPVTWGTSSRDEDLENCSHHL (SEQ ID NO: 155). In some embodiments, the IL-15Ra or the functional fragment thereof comprises an amino acid sequence having at least 90%, 91%, 92%, 93%, 94%, 95%, 96%, 97%, 98%, 99% or 100% identity to ITCPPPMSVEHADIWVKSYSLYSRERYICNSGFKRKAGTSSLTECVLNKATNVAHWTTPSLKCIRDPALVHQRPAPP STVTTAGVTPQPESLSPSGKEPAASSPSSNNTAATTAAIVPGSQLMPSKSPSTGTTEISSHESSHGTPSQTTAKNWE LTASASHQPPGVYPQGHSDTTVAISTSTVLLCGLSAVSLLACYLKSRQTPPLASVEMEAMEALPVTWGTSSRDEDLE NCSHHL (SEQ ID NO: 156). In some embodiments, the IL-15Ra or the functional fragment thereof comprises an amino acid sequence having at least 90%, 91%, 92%, 93%, 94%, 95%, 96%, 97%, 98%, 99% or 100% identity to ITCPPPMSVEHADIWVKSYSLYSRERYICNSGFKRKAGTSSLTECVLNKATNVAHWTTPSLKCIRDPALVHQRPAPP STVTTAGVTPQPESLSPSGKEPAASSPSSNNTAATTAAIVPGSQLMPSKSPSTGTTEISSHESSHGTPSQTTAKNWE LTASASHQPPGVYPQGHSDTT (SEQ ID NO: 157).

In some embodiments, the exogenous polypeptide is a transmembrane polypeptide comprising IL-15 or a functional fragment thereof. In some embodiments, the transmembrane protein further comprises a sushi domain of IL-15 receptor alpha. In some embodiments, the sushi domain of IL-15Ra comprises an amino acid sequence having at least 90%, 91%, 92%, 93%, 94%, 95%, 96%, 97%, 98%, 99% or 100% identity to TTCPPPVSIEHADIRVKNYSVNSRERYVCNSGFKRKAGTSTLIECVINKNTNVAHWTTPSLKCIR (SEQ ID NO:158). In some embodiments, the exogenous polypeptide comprises an amino acid sequence having at least 90°/%, 91%, 92%, 93%, 94%, 95%, 96%, 97%, 98%, 99% or 100% identity to MDWTWILFLVAAATRVHSNWVNVISDLKKIEDLIQSMHIDATLYTESDVHPSCKVTAMKCFLLELQVISLESGDASI HDTVENLIILANNSLSSNGNVTESGCKECEELEEKNIKEFLQSFVHIVQMFINTSTTTPAPRPPTPAPTIASQPLSL RPEACRPAAGGAVHTRGLDFACDIYIWAPLAGTCGVLLLSLVITLYC (SEQ ID NO: 159; IgE signal peptide-IL-15-CD8a transmembrane and hinge domains). In some embodiments, the exogenous polypeptide comprises an amino acid sequence having at least 90°/%, 91%, 92%, 93%, 94%, 95%, 96%, 97%, 98%, 99% or 100% identity to NWVNVISDLKKIEDLIQSMHIDATLYTESDVHPSCKVTAMKCFLLELQVISLESGDASIHDTVENLIILANNSLSSN GNVTESGCKECEELEEKNIKEFLQSFVHIVQMFINTSTTTPAPRPPTPAPTIASQPLSLRPEACRPAAGGAVHTRGL DFACDIYIWAPLAGTCGVLLLSLVITLYC (SEQ ID NO: 160; IL-15-CD8a transmembrane and hinge domains).

In some embodiments, the exogenous polypeptide is a fusion polypeptide comprising IL-15 or a functional fragment thereof and IL-15Ra or a functional fragment thereof. In some embodiments, the exogenous polypeptide comprises an amino acid sequence having at least 90%, 91%, 92%, 93%, 94%, 95%, 96%, 97%, 98%, 99% or 100% identity to MDWTWILFLVAAATRVHSNWVNVISDLKKIEDLIQSMHIDATLYTESDVHPSCKVTAMKCFLLELQVISLESGDASI HDTVENLIILANNSLSSNGNVTESGCKECEELEEKNIKEFLQSFVHIVQMFINTSSGGGSGGGGSGGGGSGGGGSGG GGSGGGGSGGGITCPPPMSVEHADIWVKSYSLYSRERYICNSGFKRKAGTSSLTECVLNKATNVAHWTTPSLKCIRD PALVHQRPAPPSTVTTAGVTPQPESLSPSGKEPAASSPSSNNTAATTAAIVPGSQLMPSKSPSTGTTEISSHESSHG TPSQTTAKNWELTASASHQPPGVYPQGHSDTTVAISTSTVLLCGLSAVSLLACYLKSRQTPPLASVEMEAMEALPVT WGTSSRDEDLENCSHHL (SEQ ID NO: 161; IgE signal peptide-IL-15-SG₃-(SG₄)₅-SG₃-IL-15Ra). In some embodiments, the exogenous polypeptide comprises an amino acid sequence having at least 90%, 91%, 92%, 93%, 94%, 95%, 96%, 97%, 98%, 99% or 100% identity to NWVNVISDLKKIEDLIQSMHIDATLYTESDVHPSCKVTAMKCFLLELQVISLESGDASIHDTVENLIILANNSLSSN GNVTESGCKECEELEEKNIKEFLQSFVHIVQMFINTSSGGGSGGGGSGGGGSGGGGSGGGGSGGGGSGGGITCPPPM SVEHADIWVKSYSLYSRERYICNSGFKRKAGTSSLTECVLNKATNVAHWTTPSLKCIRDPALVHQRPAPPSTVTTAG VTPQPESLSPSGKEPAASSPSSNNTAATTAAIVPGSQLMPSKSPSTGTTEISSHESSHGTPSQTTAKNWELTASASH QPPGVYPQGHSDTTVAISTSTVLLCGLSAVSLLACYLKSRQTPPLASVEMEAMEALPVTWGTSSRDEDLENCSHHL (SEQ ID NO: 162; IL-15-SG₃-(SG₄)₅-SG₃-IL-15Ra). In some embodiments, the exogenous polypeptide comprises an amino acid sequence having at least 90%, 91%, 92%, 93%, 94%, 95%, 96%, 97%, 98%, 99% or 100% identity to MDWTWILFLVAAATRVHSNWVNVISDLKKIEDLIQSMHIDATLYTESDVHPSCKVTAMKCFLLELQVISLESGDASI HDTVENLIILANNSLSSNGNVTESGCKECEELEEKNIKEFLQSFVHIVQMFINTSGSGSGSGSGSGSGSGSGSGSGS GSGSGSGSVAISTSTVLLCGLSAVSLLACYLKSRQTPPLASVEMEAMEALPVTWGTSSRDEDLENCSHHL (SEQ ID NO: 163; IgE signal peptide-IL-15-(GS)₁₅-IL-15Ra (206-267)). In some embodiments, the exogenous polypeptide comprises an amino acid sequence having at least 90%, 91%, 92%, 93%, 94%, 95%, 96%, 97%, 98%, 99% or 100% identity to MDWTWILFLVAAATRVHSNWVNVISDLKKIEDLIQSMHIDATLYTESDVHPSCKVTAMKCFLLELQVISLESGDASI HDTVENLIILANNSLSSNGNVTESGCKECEELEEKNIKEFLQSFVHIVQMFINTSGSGSGSGSGSGSGSGSGSGSGS GSGSGSGSVAISTSTVLLCGLSAVSLLACYLKSRQTPPLASVEMEAMEALPVTWGTSSRDEDLENCSHHL (SEQ ID NO: 164; IL-15-(GS)₁₅-IL-15Ra (206-267)).

In some embodiments, the one or more exogenous polypeptides is transmembrane polypeptide comprising IL-15 or a functional fragment thereof, the sushi domain of IL-15 receptor alpha or a functional fragment thereof, and a transmembrane domain (e.g., a transmembrane domain provided herein). In some embodiments, exogenous polypeptide comprises a linker disposed between the IL-15 or the functional fragment thereof and the sushi domain of IL-15 receptor alpha or the functional fragment thereof. In some embodiments, exogenous polypeptide includes a linker disposed between the IL-15 or the functional fragment thereof and the sushi domain of the IL-15 receptor alpha or the functional fragment thereof, as well as a linker disposed between the sushi domain of the IL-15 receptor alpha or the functional fragment thereof and the transmembrane domain. In some embodiments, the linker comprises or consists of 1-20 amino acid residues in length (e.g., 1, 2, 3, 4, 5, 6, 7, 8, 9, 10, 11, 12, 13, 14, 15, 16, 17, 18, 19, or 20 amino acids in length). In some embodiments, the linker comprises or consists of between about 5 and about 25 amino acids in length (e.g., between about 5 and about 20 amino acids in length, between about 10 and about 25 amino acids in length, or between about 10 and about 20 amino acids in length). In some embodiments, the linker is non-immunogenic. In some embodiments, the linker is one of the linkers provided elsewhere herein. Table 5 provides additional examples of linkers that may be used. In some embodiments, the linker comprises or consists of an amino acid sequence provided in Table 1. In some embodiments, the linker comprises or consists of SEQ ID NOs: 165-179.

TABLE 5
Examples of linker sequences

| | SEQ |
|---|---|
| | ID |
| Amino Acid Sequence | NO: |
| GGGGS | 165 |
| GGGGSGGGGSGGGGS | 166 |
| GGSGGSGGYPYDVPDYAGGGSGGGS | 167 |
| GGSGGSGGGGGSGGGSGGGSGGGS | 168 |
| GGSGGSGGGPEDEPGSGSGGGSGGGS | 169 |
| GGSGGSGGGGGSGGGSGGGSGGGSGSGSGSGSEDGSGSGSGS | 170 |
| GSGSGSGSGSEDEDEDEDGSGSGSGSGS | 171 |
| GGGGSGGGGSGGGGSGGGGS | 172 |
| GSGSGSGSEDGSGSGSGS | 173 |
| GSGSGSGSGSGSGSGSGS | 174 |
| GCGGSGGGGSGGGGS | 175 |
| GGSGGSGGGGGSGGGSGGGSGGGS | 176 |
| SGRGGGGSGGGGSGGGGSGGGGSSPA | 177 |
| GGGGSGGGGSGGGGSGGGGGGGG | 178 |
| SGRGASSGSSGSGSQKKPRYEIRWKVVVISAILALVVLTVISLIILIMLWGSGMQSPA | 179 |

In some embodiments, the IL-15 or a functional fragment thereof and the IL-15Ra or a functional fragment thereof are separated by a cleavage sequence (e.g., a 2A sequence (e.g., a T2A cleavage sequence (GSGEGRGSLLTCGDVEENPGP (SEQ ID NO: 180)), a P2A cleavage sequence (GSGATNFSLLKQAGDVEENPGP (SEQ ID NO: 181)), a E2A cleavage sequence (GSGQCTNYALLKLAGDVESNPGP (SEQ ID NO: 182)) or a F2A cleavage sequence GSGVKQTLNFDLLKLAGDVESNPGP (SEQ ID NO: 183)).

In some embodiments, the exogenous polypeptide is a TGF-β dominant negative receptor. Without wishing to be bound by theory, inclusion of a TGF-β dominant negative receptor in the immune cells described herein may allow for the immune cells to have enhanced ability to overcome the tumor microenvironment in vivo. In some embodiments, the TGF-β dominant negative receptor comprises the extracellular domain of a TGFβ receptor (e.g., the extracellular domain of human TGF-beta receptor (TGFBR) (e.g., TGF-beta receptor 1 (TGFBR1) and/or TGF-beta receptor 2 (TGFBR2)) and the transmembrane domain of a TGFβ receptor (e.g., the transmembrane domain of TGFBR1 or TGFBR2). In some embodiments, the TGF-β dominant negative receptor includes a transmembrane domain of human TGFBR1 (e.g., AAVIAGPVCFVCISLMLMVYI (SEQ ID NO: 191)). In some embodiments, the TGF-β dominant negative receptor includes a transmembrane domain of human TGFBR2 (e.g., VTGISLLPPLGVAISVIIIFY (SEQ ID NO: 192)). In some embodiments, a TGF-β dominant negative receptor comprises the extracellular domain of a TGFβ receptor (e.g., the extracellular domain of TGFBR1 or TGFBR2) and a heterologous transmembrane domain (e.g., any of the transmembrane domains provided herein (e.g., a CD28 transmembrane domain)). In some embodiments, the TGF-β dominant negative receptor includes a transmembrane domain of human CD28 (e.g., FWVLVVVGGVLACYSLLVTVAFIIFWV (SEQ ID NO: 120). In some embodiments, the TGF-β dominant negative receptor includes a transmembrane domain of human CD8α (IYIWAPLAGTCGVLLLSLVIT (SEQ ID NO: 118)).

In some embodiments, the TGF-β dominant negative receptor includes the extracellular domain of a TGFBR2. In some embodiments, the TGF-β dominant negative receptor comprises an amino acid sequence having at least 90%, 91%, 92%, 93%, 94%, 95%, 96%, 97%, 98%, 99% or 100% identity to GRGLLRGLWPLHIVLWTRIASTIPPHVQKSVNNDMIVTDNNGAVKFPQLCKFCDVRFSTCDNQKSCMSNCSITSICE KPQEVCVAVWRKNDENITLETVCHDPKLPYHDFILEDAASPKCIMKEKKKPGETFFMCSCSSDECNDNIIFSEEYNT SNPDLLLVIFQ (SEQ ID NO: 184). In some embodiments, the TGF-β dominant negative receptor comprises an amino acid sequence having at least 90%, 91%, 92%, 93%, 94%, 95%, 96%, 97%, 98%, 99% or 100% identity to TIPPHVQKSVNNDMIVTDNNGAVKFPQLCKFCDVRFSTCDNQKSCMSNCSITSICEKPQEVCVAVWRKNDENITLET VCHDPKLPYHDFILEDAASPKCIMKEKKKPGETFFMCSCSSDECNDNIIFSEEYNTSNPDLLLVIFQ (SEQ ID NO: 185). In some embodiments, the TGF-β dominant negative receptor comprises an amino acid sequence having at least 90%, 91%, 92%, 93%, 94%, 95%, 96%, 97%, 98%, 99% or 100% identity to MGRGLLRGLWPLHIVLWTRIASTIPPHVQKSVNNDMIVTDNNGAVKFPQLCKFCDVRFSTCDNQKSCMSNCSITSIC EKPQEVCVAVWRKNDENITLETVCHDPKLPYHDFILEDAASPKCIMKEKKKPGETFFMCSCSSDECNDNIIFSEEYN TSNPDLLLVIFQVTGISLLPPLGVAISVIIIFYCYRVNRQQKLSS (SEQ ID NO: 186).

In some embodiments, the TGF-β dominant negative receptor comprises an amino acid sequence having at least 90%, 91%, 92%, 93%, 94%, 95%, 96%, 97%, 98%, 99% or 100% identity to TIPPHVQKSVNNDMIVTDNNGAVKFPQLCKFCDVRFSTCDNQKSCMSNCSITSICEKPQEVCVAVWRKNDENITLET VCHDPKLPYHDFILEDAASPKCIMKEKKKPGETFFMCSCSSDECNDNIIFSEEYNTSNPDLLLVIFQVTGISLLPPL GVAISVIIIFYCYRVNRQQKLSS (SEQ ID NO: 187). In some embodiments, the TGF-β dominant negative receptor comprises an amino acid sequence having at least 90%, 91%, 92%, 93%, 94%, 95%, 96%, 97%, 98%, 99% or 100% identity to MGRGLLRGLWPLHIVLWTRIASTIPPHVQKSVNNDMIVTDNNGAVKFPQLCKFCDVRFSTCDNQKSCMSNCSITSIC EKPQEVCVAVWRKNDENITLETVCHDPKLPYHDFILEDAASPKCIMKEKKKPGETFFMCSCSSDECNDNIIFSEEYN TSNPDLLLVIFQFWVLVVVGGVLACYSLLVTVAFIIFWVCYRVNRQQKLSS (SEQ ID NO: 188). In some embodiments, the TGF-β dominant negative receptor comprises an amino acid sequence having at least 90%, 91%, 92%, 93%, 94%, 95%, 96%, 97%, 98%, 99% or 100% identity to TIPPHVQKSVNNDMIVTDNNGAVKFPQLCKFCDVRFSTCDNQKSCMSNCSITSICEKPQEVCVAVWRKNDENITLET VCHDPKLPYHDFILEDAASPKCIMKEKKKPGETFFMCSCSSDECNDNIIFSEEYNTSNPDLLLVIFQFWVLVVVGGV LACYSLLVTVAFIIFWVCYRVNRQQKLSS (SEQ ID NO: 189).

In some embodiments, the TGF-β dominant negative receptor includes the extracellular domain of a TGF-β type I receptor. In some embodiments, the TGF-β dominant negative receptor comprises an amino acid sequence having at least 90%, 91%, 92%, 93%, 94%, 95%, 96%, 97%, 98%, 99% or 100% identity to LQCFCHLCTKDNFTCVTDGLCFVSVTETTDKVIHNSMCIAEIDLIPRDRPFVCAPSSKTGSVTTTYCCNQDHCNKIE LPTTVKSSPGLGPVEL (SEQ ID NO: 190).

In some embodiments, the immune cells provided herein (e.g., T cells and NK cells) are engineered to express one or more additional exogenous polypeptides. The one or more additional exogenous polypeptides include, but are not limited to, cytokines, chemokines, and other molecules that contribute to the persistence, activation, and proliferation of the cells.

In some embodiments, the immune cells of the present disclosure may comprise one or more safety switch polypeptide or kill-switch genes (e.g., caspase-9, inducible FAS (iFAS), and inducible caspase-9 (iCASP9)). In some embodiments, the presence of a safety switch polypeptide or kill-switch gene may be used to ablate the immune cells in vivo following administration to a subject. In some instances, the safety switch protein expression is conditionally regulated. This conditional regulation could be variable and be regulated using small molecule-mediated post-translational activation and tissue-specific and/or temporal transcriptional regulation. The safety switch could mediate induction of apoptosis, inhibition of protein synthesis or DNA replication, growth arrest, transcriptional and post-transcriptional genetic regulation and/or antibody-mediated depletion in the immune cell. In some embodiments, the safety switch polypeptide is activated by an exogenous molecule, e.g., a prodrug.

Kill switch genes are generally genes which, upon administration of a prodrug, effects transition of a gene product to a compound which induced cell death in the cell containing the gene. Examples of kill switch gene/prodrug combinations which may be used include, but are not limited to inducible caspase 9 (iCASP9) and rimiducid; RQR8 and rituximab; truncated version of EGFR variant III (EGFRv3) and cetuximab; herpes simplex virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside; and Escherichia coli purine nucleoside phosphorylase and purine 6-methylpurine.

The present disclosure also provides a population of engineered immune cells (and compositions including the population of engineered immune cells), wherein a plurality of the engineered immune cells of the population comprise any of the CARs disclosed herein. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the immune cells in the population comprise the CAR. In some embodiments, a nucleic acid encoding the CAR is integrated into the genome of an engineered immune cell at a copy number of at least 1, 2, 3, 4, 5, 6, 7, 8, 9, 10, 20 or 30 copies per cell.

In some embodiments, the engineered immune cells are engineered to express one or more exogenous polypeptides provided herein (e.g., an exogenous polypeptide comprising IL-15 or a functional fragment thereof and a TGF-β dominant negative receptor). In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the immune cells in the population comprise the one or more exogenous polypeptide(s). In some embodiments, a nucleic acid encoding the one or more exogenous polypeptide(s) is integrated into the genome of an engineered immune cell at a copy number of at least 1, 2, 3, 4, 5, 6, 7, 8, 9, 10, 20 or 30 copies per cell.

In some embodiments, the engineered immune cells (e.g., NK cells and T cells) provided herein (e.g., expressing a CAR provided herein and, optionally one or more exogenous polypeptides) may exhibit one or more desirable functions (e.g., effector functions). In some embodiments, the engineered immune cells exhibit cytotoxicity to mesothelin-expressing cells (e.g., mesothelin expressing cancer cells). In some embodiments, the engineered immune cells (e.g., NK cells) exhibit directed secretion of cytolytic granules or engagement of death domain-containing receptors. In some embodiments, the cytolytic granules comprise perforin and/or granzymes. In some embodiments, the engineered immune cells are NK cells comprising one or more of the following functions: degranulation (e.g., CD107a expression), activation (e.g., CD69 production), cytokine production (e.g., TNFalpha or IFNgamma production), target cell line killing, and efficacy in mouse models. Illustrative assays for measuring NK cell cytotoxicity and CD107a (granule release) are provided in Li et al., Cell Stem Cell 23:181-92, 2018.

### Nucleic Acids and Methods of Making Engineered Immune Cells

Included in the scope of the disclosure are nucleic acid sequences encoding any of the CARs described herein, and optionally, any one of the other described polypeptides. In some embodiments, the nucleic acid molecule encodes a CAR comprising an amino acid sequence that is at least 80%, at least 85%, at least 90%, at least 95%, at least 96%, at least 97%, at least 98%, at least 99%, or 100% identical to any one of SEQ ID NOs: 1-90. In some embodiments, the nucleic acid molecule further encodes at least one exogenous polypeptide provided herein.

Methods known in the art may be used construct a vector comprising a nucleic acid encoding a CAR and/or an exogenous polypeptide(s) described herein using standard recombinant techniques (see, for example, Sambrook et al., 2001 and Ausubel et al, 1994, supra). Vectors include but are not limited to, plasmids, cosmids, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV, etc.), lentiviral vectors (e.g., derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

In some embodiments, the methods of the disclosure include introducing a nucleic acid and/or a genomic editing of an immune cell ex vivo, in vivo, in vitro, or in situ using a vector or a plurality of vectors. In some embodiments, the vector is a viral vector. In some embodiments, the viral vector is a non-integrating non-chromosomal vector. Exemplary non-integrating non-chromosomal vectors include, but are not limited to, adeno-associated virus (AAV), adenovirus, and herpes viruses. In some embodiments, the viral vector is an integrating chromosomal vector. Integrating chromosomal vectors include, but are not limited to, adeno-associated vectors (AAV), lentiviruses, and gamma-retroviruses (e.g., Murine Leukemia Virus (MLV), Spleen-Focus Forming Virus (SFFV), and Myeloproliferative Sarcoma Virus (MPSV), and vectors derived therefrom). Exemplary vector combinations include: a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

Exogenous nucleic acids encoding a CAR and/or one or more exogenous polypeptides described herein may be introduced into an immune cell using transient integration, random integration, site-specific integration (e.g., assisted or non-assisted site-specific integration) or biased integration. In some embodiments, transgenes with homologous nucleotide extensions enable genomic integration by homologous recombination, microhomology-mediated end joining, or nonhomologous end-joining are used. In some embodiments the site-specific integration occurs at a safe harbor site (e.g., intronic sequences of the human albumin gene, the adeno-associated virus site 1 (AAVS1), a naturally occurring site of integration of AAV on chromosome 19, the chemokine (C-C motif) receptor 5 (CCR5) gene locus, and the human ortholog of the mouse Rosa26 locus). In some embodiments, site-specific integration occurs at a site that disrupts expression of a target gene (e.g., an immunosuppressive gene and a gene involved in allo-rejection).

Expression cassettes included in vectors of the present disclosure may contain (in a 5′-to-3′ direction) a eukaryotic transcriptional promoter operably linked to a protein-coding sequence, splice signals including intervening sequences, and a transcriptional termination/polyadenylation sequence. The promoter may be a constitutive, inducible, and/or tissue-specific promoter. Promoters may or may not be used in conjunction with an enhancer element. Any promoter/enhancer combination can also be used to drive protein expression. Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter, GADPH promoter, metallothionein promoter; and concatenated response element promoters, such as cyclic AMP response element promoters (ere), serum response element promoter (sre), phorbol ester promoter (TPA) response element promoters (tre), CMV 1E promoter, dectin-1 promoter, dectin-2 promoter, human CD11c promoter, SV40 promoter, Ad MLP promoter, beta-actin promoter, MHC class I or MHC class II promoters.

In certain embodiments, internal ribosome entry sites (IRES) elements are used to create multigene, or polycistronic, messages. IRES elements can be linked to heterologous open reading frames. Multiple open reading frames can be transcribed together, each separated by an IRES, creating polycistronic messages. Additionally, certain 2A cleavage sequence elements could be used to create linked- or co-expression of genes in the constructs provided in the present disclosure. For example, cleavage sequences could be used to co-express genes by linking open reading frames to form a single cistron.

In addition to viral delivery of the nucleic acids encoding one or more polypeptides described herein, any suitable methods for nucleic acid delivery for transformation of a cell may be used, as described herein or as would be known to one of ordinary skill in the art. Such methods include, but are not limited to, direct delivery of DNA such as by ex vivo transfection, by injection, including microinjection; by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s), or organism(s) may be stably or transiently transformed.

In some embodiments, transposons or viral integration systems may be used. In some embodiments, a transposon may be present in an expression vector (e.g., a DNA plasmid or mini-circle vector). Exemplary transposons include, but are not limited to, piggyBac, hyperactive piggyBac, Sleeping Beauty (SB), hyperactive Sleeping Beauty (SB100x), SBI11, SB110, Tn7, TcBuster, hyperactive TcBuster, Frog Prince, IS5, Tn10, Tn903, SPIN, hAT, Hermes, Hobo, AeBuster1, AeBuster2, AeBuster3, BtBuster1, BtBuster2, CfBuster1, CfBuster2, Tol2, mini-Tol2, Tc3, Mos1, MuA, Himar I, Helitron and engineered versions of transposase family enzymes (Zhang et al., PLoS Genet. 5:e1000689, 2009; Wilson et al., J. Microbiol. Methods 71:332-5, 2007; the entire contents of which are incorporated by reference herein). Exemplary transposons also include the transposons described in Arensburger et al. (Genetics 188(1):45-57, 2011; the entire contents of which are incorporated by reference herein), or a SPACE INVADERS (SPIN) transposon (see, e.g., Pace et al., Proc. Natl. Acad. Sci. U.S.A. 105(44):17023-17028, 2008; the entire contents of which are incorporated by reference herein). In some embodiments, the gene transfer system can be delivered to the cell encoded in DNA, encoded in mRNA, as a protein, or as a nucleoprotein complex. Alternatively, the gene transfer system can be integrated into the genome of a host cell using, for example, a retro-transposon, random plasmid integration, recombinase-mediated integration, homologous recombination mediated integration, or non-homologous end joining mediated integration. More examples of transposition systems that can be used with certain embodiments of the compositions and methods provided herein include Staphylococcus aureus Tn552 (Colegio et al., J. Bacteriol. 183:2384-8, 2001; Kirby et al., Mol. Microbiol. 43:173-86, 2002), Tyl (Devine & Boeke, Nucleic Acids Res. 22:3765-72, 1994 and International Publication WO 95/23875), Transposon Tn7 (Craig, Science 271:1512, 1996; Craig, Review in: Curr. Top. Microbiol. Immunol. 204:27-48, 1996), Tn/O and IS10 (Kleckner et al., Curr. Top. Microbiol. Immunol. 204:49-82, 1996), Mariner transposase (Lampe et al., EMBO J. 15:5470-9, 1996), Tel (Plasterk, Curr. Topics Microbiol. Immunol. 204:125-43, 1996), P Element (Gloor, Methods Mol. Biol. 260:97-114, 2004), Tn3 (Ichikawa & Ohtsubo, J. Biol. Chem. 265:18829-32, 1990), bacterial insertion sequences (Ohtsubo & Sekine, Curr. Top. Microbiol. Immunol. 204:1-26, 1996), retroviruses (Brown et al., Proc. Natl. Acad. Sci. U.S.A. 86:2525-9, 1989), and retrotransposon of yeast (Boeke & Corces, Ann. Rev. Microbiol. 43:403-34, 1989). The entire contents of each of the foregoing references are incorporated by reference herein.

Exemplary TcBuster family transposons include, but are not limited to Ac-like (AAC46515), Ac (CAA29005), AeBuster1 (ABF20543), AeBuster2 (ABF20544), AmBuster1 (EFB22616), AmBuster2 (EFB25016), AmBuster3 (EFB20710), AmBuster4 (EFB22020), BtBuster1 (ABF22695), BtBuster2 (ABF22700), BtBuster3 (ABF22697), CfBuster1 (ABF22696), CfBuster2 (ABF22701), CfBuster3 (XP_854762), CfBuster4 (XP_545451), CsBuster (ABF20548), Daysleeper (CAB68118), DrBuster1 (ABF20549), DrBuster2 (ABF20550), EcBuster1 (XP_001504971), EcBuster3 (XP_001503499), EcBuster4 (XP_001504928), Hermes (AAC37217), hermit (LCU22467), Herves (AAS21248), hobo (A39652), Homer (AAD03082), hopper-we (AAL93203), HsBuster1 (AAF18454), HsBuster2 (ABF22698), HsBuster3 (NP_071373), HsBuster4 (AAS01734), IpTip100 (BAA36225), MamBuster2 (XP_001108973), MamBuster3 (XP_001084430), MamBuster3 (XP_001084430), MamBuster4 (XP_001101327), MmBuster2 (AAF18453), PtBuster2 (ABF22699), PtBuster3 (XP_001142453), PtBuster4 (XP_527300), Restless (CAA93759), RnBuster2 (NP_001102151), SpBuster1 (ABF20546), SpBuster2 (ABF20547), SsBuster4 (XP_001929194), Tam3 (CAA38906), TcBuster (ABF20545), Tol2 (BAA87039), tramp (CAA76545), XtBuster (ABF20551), ENSEMBL (sequences available on the World Wide Web at ensembl.org), PtBuster1 (ENSPTRG00000003364), REPBASE (sequences available on the World Wide Web at girinst.org), Ac-like2 (hAT-7_DR), Ac-like1 (hAT-6_DR), hAT-5_DR (hAT-5_DR), MlBuster1 (hAT-4_ML), Myotis-hAT1 (Myotis-hAT1), SPIN_Et (SPIN_Et), SPIN_Ml (SPIN_Ml), SPIN-Og (SPIN-Og), TEFam (sequences available on the World Wide Web at tefam.biochem.vt.edu), AeHermes1 (TF0013337), AeBuster3 (TF001186), AeBuster4 (TF001187), AeBuster5 (TF001188), AeBuster7 (TF001336), AeHermes2 (TF0013338), AeTip100-2 (TF000910), Cx-Kink2 (TF001637), Cx-Kink3 (TF001638), Cx-Kink4 (TF001639), Cx-Kink5 (TF001640), Cx-Kink7 (TF001636), and Cx-Kink8 (TF001635).

Compositions and methods of the disclosure may comprise a TcBuster transposon and/or a TcBuster transposase.

Compositions and methods of the disclosure may comprise a TcBuster transposon and/or a hyperactive TcBuster transposase. A hyperactive TcBuster transposase may have increased excision and/or increased insertion frequency when compared to an excision and/or insertion frequency of a wild type TcBuster transposase. In some embodiments, a TcBuster transposase may comprise any of the mutations disclosed in WO 2019/246486, which is incorporated herein by reference in its entirety.

In some embodiments, a recombinant immune cell produced by transposition-based methods may comprise sequences flanking the nucleotide sequence incorporated into the cell's genome by transposition. Illustrative examples of such flanking sequences (also known as excision footprints) are provided in Woodard et al., PLoS ONE 7(11):e42666, 2012.

In some embodiments of the methods of the disclosure, a modified immune cell of the disclosure may be produced by introducing a transgene into an immune cell of the disclosure. The introducing step may comprise delivery of a nucleic acid sequence and/or a genomic editing construct via a non-transposition delivery system. In some embodiments, a nucleic acid and/or a genomic editing construct may be introduced into an immune cell ex vivo, in vivo, in vitro, or in situ comprising one or more of topical delivery, adsorption, absorption, electroporation, spin-fection, co-culture, transfection, mechanical delivery, sonic delivery, vibrational delivery, magnetofection, by nanoparticle-mediated delivery, liposomal transfection, calcium phosphate transfection, fugene transfection, dendrimer-mediated transfection, cell squeezing, cell bombardment, or gene gun techniques, liposomal delivery, delivery by micelles, and delivery by polymerosomes.

In some embodiments of the methods of the disclosure, a nucleic acid and/or a genomic editing construct is introduced into an immune cell ex vivo, in vivo, in vitro or in situ comprising a non-viral vector. In some embodiments, the non-viral vector comprises plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA).

In some embodiments of the methods of the disclosure, enzymes may be used to create strand breaks in the host genome to facilitate delivery or integration of the transgene. In some embodiments, the enzymes create single-strand breaks. In some embodiments, the enzymes create double-strand breaks. Examples of break-inducing enzymes include but are not limited to: transposases, integrases, endonucleases, meganucleases, megaTALs, CRISPR-Cas9, transcription activator-like effector nucleases (TALEN) or zinc finger nucleases (ZFN). Other editing or break-inducing enzymes may include, without limitation, nucleases such as Cas12a (includes MAD7), Cas12b, Cas12c, Cas13, and many more. In certain instance, the Cas12a nuclease is MAD7. In some embodiments, break-inducing enzymes can be delivered to the cell encoded in DNA, encoded in mRNA, as a protein, as a nucleoprotein complex with a guide RNA (gRNA).

In some embodiments of the methods of the disclosure, the site-specific transgene integration is controlled by a vector-mediated integration site bias. In some embodiments vector-mediated integration site bias is controlled by the chosen lentiviral vector. In some embodiments vector-mediated integration site bias is controlled by the chosen gamma-retroviral vector.

In some embodiments of the methods of the disclosure, the site-specific transgene integration site is a non-stable chromosomal insertion. In some embodiments, the integrated transgene may become silenced, removed, excised, or further modified.

In some embodiments of the methods of the disclosure, the genome modification is a non-stable integration of a transgene. In some embodiments, the non-stable integration can be a transient non-chromosomal integration, a semi-stable non chromosomal integration, a semi-persistent non-chromosomal insertion, or a non-stable chromosomal insertion. In some embodiments, the transient non-chromosomal insertion can be epi-chromosomal or cytoplasmic.

In some embodiments, the transient non-chromosomal insertion of a transgene does not integrate into a chromosome and the modified genetic material is not replicated during cell division.

In some embodiments of the methods of the disclosure, the genome modification is a semi-stable or persistent non-chromosomal integration of a transgene. In some embodiments, a DNA vector encodes a Scaffold/matrix attachment region (S-MAR) module that binds to nuclear matrix proteins for episomal retention of a non-viral vector allowing for autonomous replication in the nucleus of dividing cells.

In some embodiments of the methods of the disclosure, the genome modification is a non-stable chromosomal integration of a transgene. In some embodiments, the integrated transgene may become silenced, removed, excised, or further modified.

In some embodiments of the methods of the disclosure, the modification to the genome by transgene insertion can occur via host cell-directed double-strand breakage repair (homology-directed repair) by homologous recombination (HR), microhomology-mediated end joining (MMEJ), nonhomologous end joining (NHEJ), transposase enzyme-mediated modification, integrase enzyme-mediated modification, endonuclease enzyme-mediated modification, or recombinant enzyme-mediated modification. In some embodiments, the modification to the genome of the immune cell by transgene insertion can occur using a nuclease described herein (e.g., CRISPR-Cas9, TALEN and ZFNs).

### Gene Editing

The term “gene editing” as used herein refers to the insertion, deletion, or replacement of nucleic acids in genomic DNA so as to add, disrupt or modify the function of the product that is encoded by a gene. Various gene editing systems require, at a minimum, the introduction of a cutting enzyme (e.g., a nuclease or recombinase) that cuts genomic DNA to disrupt or activate gene function.

Further, in gene editing systems that involve inserting new or existing nucleotides/nucleic acids, insertion tools (e.g., DNA template vectors, transposable elements (transposons or retrotransposons) must be delivered to the cell in addition to the cutting enzyme (e.g., a nuclease, recombinase, integrase, or transposase). Examples of such insertion tools for a recombinase may include a DNA vector. Other gene editing systems require the delivery of an integrase along with an insertion vector, a transposase along with a transposon/retrotransposon, etc. In some embodiments, an example recombinase that may be used as a cutting enzyme is the CRE recombinase. In various embodiments, example integrases that may be used in insertion tools include viral based enzymes taken from any of a number of viruses including, but not limited to, AAV, gamma retrovirus, and lentivirus. Example transposons/retrotransposons that may be used in insertion tools include, but are not limited to, the piggyBac® transposon, Sleeping Beauty transposon, TcBuster transposon and the L1 retrotransposon.

In certain embodiments of the methods of the disclosure, non-viral vectors are used for transgene delivery. In certain embodiments, the non-viral vector is a nucleic acid. In certain embodiments, the nucleic acid non-viral vector is plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). In certain embodiments, the non-viral vector is a transposon. In certain embodiments, the transposon is TcBuster.

In certain embodiments of the methods of the disclosure, transgene delivery can occur via viral vector. In certain embodiments, the viral vector is a non-integrating non-chromosomal vector. Non-integrating non-chromosomal vectors can include adeno-associated virus (AAV), adenovirus, and herpes viruses. In certain embodiments, the viral vector is an integrating chromosomal vector. Integrating chromosomal vectors can include adeno-associated vectors (AAV), lentiviruses, and gamma-retroviruses.

In certain embodiments of the methods of the disclosure, transgene delivery can occur by a combination of vectors. Exemplary but non-limiting vector combinations can include: viral plus non-viral vectors, more than one non-viral vector, or more than one viral vector. Exemplary but non-limiting vectors combinations can include DNA-derived plus RNA-derived vectors, RNA plus reverse transcriptase, a transposon and a transposase, a non-viral vectors plus an endonuclease, and a viral vector plus an endonuclease.

In certain embodiments of the methods of the disclosure, the genome modification can be a stable integration of a transgene, a transient integration of a transgene, a site-specific integration of a transgene, or a biased integration of a transgene.

In certain embodiments of the methods of the disclosure, the genome modification can be a stable chromosomal integration of a transgene. In certain embodiments, the stable chromosomal integration can be a random integration, a site-specific integration, or a biased integration. In certain embodiments, the site-specific integration can be non-assisted or assisted. In certain embodiments, the assisted site-specific integration is co-delivered with a site-directed nuclease. In certain embodiments, the site-directed nuclease comprises a transgene with 5′ and 3′ nucleotide sequence extensions that contain homology to upstream and downstream regions of the site of genomic integration. In certain embodiments, the transgene with homologous nucleotide extensions enables genomic integration by homologous recombination, microhomology-mediated end joining, or nonhomologous end-joining. In certain embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C-C motif) receptor 5 (CCR5) gene and the site of the human ortholog of the mouse Rosa26 locus.

In certain embodiments, the site-specific transgene integration occurs at a site that disrupts expression of a target gene. In certain embodiments, disruption of target gene expression occurs by site-specific integration at introns, exons, promoters, genetic elements, enhancers, suppressors, start codons, stop codons, and response elements. In certain embodiments, exemplary target genes targeted by site-specific integration include but are not limited to PD1, any immunosuppressive gene, and genes involved in allo-rejection.

In certain embodiments, the site-specific transgene integration occurs at a site that results in enhanced expression of a target gene. In certain embodiments, enhancement of target gene expression occurs by site-specific integration at introns, exons, promoters, genetic elements, enhancers, suppressors, start codons, stop codons, and response elements.

In certain embodiments of the methods of the disclosure, enzymes may be used to create strand breaks in the host genome to facilitate delivery or integration of the transgene. In certain embodiments, enzymes create single-strand breaks. In certain embodiments, enzymes create double-strand breaks. In certain embodiments, examples of break-inducing enzymes include but are not limited to transposases, integrases, endonucleases, meganucleases, megaTALs, CRISPR-Cas9, transcription activator-like effector nucleases (TALEN) and zinc finger nucleases (ZFN). In certain embodiments, break-inducing enzymes can be delivered to the cell encoded in DNA, encoded in mRNA, as a protein, as a nucleoprotein complex with a guide RNA (gRNA). In certain embodiments of the methods of the disclosure, the genome immune cells may be engineered by transgene insertion via host cell-directed double-strand breakage repair (homology-directed repair) by homologous recombination (HR), microhomology-mediated end joining (MMFJ), nonhomologous end joining (NHEJ), transposase enzyme-mediated modification, integrase enzyme-mediated modification, endonuclease enzyme-mediated modification, or recombinant enzyme-mediated modification. In certain embodiments, the modification to the genome by transgene insertion can occur via CRISPR-Cas9, TALEN or ZFNs.

In some embodiments, the engineered immune cells of the present disclosure may be identified in vitro or in vivo by including a marker (or a gene encoding the marker) in an exogenous nucleic acid introduced into the cell (e.g., an expression vector). For example, genes that confer resistance to neomycin, puromycin, hygromycin, DHFR, GPT, zeocin and histidinol may be used as selection markers. Other types of markers including screenable markers such as fluorescent proteins (e.g., GFP). Alternatively, screenable enzymes may be used as negative selection markers (e.g., herpes simplex virus thymidine kinase (tk) or chloramphenicol acetyltransferase (CAT)). Further examples of markers are known in the art.

The immune cells (e.g., NK cells and T cells) provided herein can also be produced using coupling reagents to link an exogenous polypeptide to the cell using a click chemistry reaction. Coupling reagents can be used to couple an exogenous polypeptide to a cell. Various forms of click chemistry reaction are known in the art, such as the Huisgen 1,3-dipolar cycloaddition copper catalyzed reaction (Tornoe et al., J. Organic Chem. 67:3057-64, 2002), which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions. In some embodiments, the click chemistry approach comprises copper catalyzed reaction, as described, e.g., in Rostovstev et al. Angew Chem Int Ed 41:2596, 2002; Tomoe et al. J. Org. Chem. 67:3057, 2002. In other embodiments, the click chemistry approach comprises copper-free click reaction, as described, e.g., by Agard et al. J. Am. Chem. Soc. 126:15046-47, 2004, and Ning et al. Angew Chem. Int. Ed. 49:3065-68, 2010.

In some embodiments, one or more exogenous polypeptides can be conjugated to the surface of an immune cell (e.g., an NK cell) by various chemical and enzymatic means, including but not limited to chemical conjugation with bifunctional cross-linking agents such as, e.g., an NHS ester-maleimide heterobifunctional crosslinker to connect a primary amine group with a reduced thiol group. These methods also include enzymatic strategies such as, e.g., transpeptidase reaction mediated by a sortase enzyme (see, e.g., Swee et al., Proc. Natl. Acad. Sci. U.S.A. 110(4):1428-33, 2013). The methods also include combination methods, such as e.g., sortase-mediated conjugation of click chemistry handles or “click handles” (an azide and an alkyne) on the exogenous polypeptide and the cell, respectively, followed by a cyclo-addition reaction to chemically bond the antigen to the cell, see e.g., Neves et al., Bioconjugate Chemistry 2013. Sortase-mediated modification of proteins is described in PCT/US2014/037545, PCT/US2014/037554, and WO 2016/014553, each of which are incorporated by reference in their entireties herein.

Following modification (e.g., genetic engineering) the immune cells may be immediately infused or may be stored. In certain aspects, following genetic modification, the cells may be propagated for days, weeks, or months ex vivo as a bulk population within about 1, 2, 3, 4, or 5 days or more following nucleic acid transfer into the immune cells. In a further aspect, the transfectants are cloned and a clone demonstrating presence of a single integrated or episomally maintained expression cassette or plasmid, and expression of the chimeric receptor is expanded ex vivo. In some embodiments, the clone is expanded at least 1,000-fold in culture. In some embodiments, the engineered immune cells are NK cells which may be expanded in the absence of feeder cells, by stimulation with IL-2, or other cytokines that bind the common gamma-chain (e.g., IL-7, IL-12, IL-15, IL-21, and others), and/or in the presence of artificial antigen presenting cells. In some embodiments, the modified immune cells may be cryopreserved.

In one embodiment of the present disclosure, the immune cells described herein are modified at a point-of-care site. In some cases, the point-of-care site is at a hospital or at a facility (e.g., a medical facility) near a subject in need of treatment. In some embodiments, the subject undergoes apheresis and peripheral blood mononuclear cells (PBMCs) or a sub population of PBMC can be enriched for example, by elutriation or Ficoll separation. Enriched PBMC or a subpopulation of PBMC can be cryopreserved in any appropriate cryopreservation solution prior to further processing. In some embodiments, the elutriation process is performed using a buffer solution containing human serum albumin. Immune cells, such as NK cells, can be isolated by selection methods described herein. The harvested immune cells can be cryopreserved in any appropriate cryopreservation solution prior to modification. The immune cells can be thawed up to 24 hours, 36 hours, 48 hours, 72 hours, or 96 hours prior to infusion. The thawed cells can be placed in cell culture buffer, for example in cell culture buffer (e.g., RPMI) supplemented with fetal bovine serum (FBS) or human serum AB or placed in a buffer that includes cytokines such as IL-2 and IL-21, prior to modification. In another aspect, the harvested immune cells can be modified immediately without cryopreservation.

In some embodiments, the population of engineered immune cells is cryopreserved prior to infusion into a subject. In some embodiments, the population of engineered immune cells is immediately infused into a subject. In some embodiments, the population of engineered immune cells is placed in a cytokine bath prior to infusion into a subject. In some embodiments, the population of engineered immune cells is cultured and/or stimulated for no more than 1, 2, 3, 4, 5, 6, 7, 14, 21, 28, 35, 42, 49, 56, 63, or 70 days. In an embodiment, a stimulation includes the co-culture of the engineered immune cells with feeder cells to promote the growth of engineered immune cells. In some instances, the engineered immune cells are not cultured ex vivo in the presence of feeder cells.

In some embodiments, the method further comprises enriching the cell population for engineered immune cells (e.g., T cells) after the immune cells are modified and/or cultured. Enrichment can include the use of fluorescence-activated cell sorting (FACS) to sort for CAR-expressing cells (e.g., using an antibody that binds to CAR described herein, a marker, or one or more exogenous polypeptides).

In some embodiments, the engineered immune cells do not undergo a propagation and activation step. In some embodiments, the engineered immune cells do not undergo an incubation or culturing step (e.g., ex vivo propagation). In certain cases, the engineered immune cells are placed in a buffer that includes IL-2 and IL-21 prior to infusion. In some embodiments, the engineered immune cells are placed or rested in cell culture buffer, for example in cell culture buffer (e.g., RPMI) prior to infusion. Prior to infusion, the engineered immune cells can be harvested, washed, and formulated in saline buffer in preparation for infusion into the subject.

### Methods of Use

In some embodiments, the present disclosure provides methods of treating a subject having a mesothelin-associated cancer comprising administering to the subject the engineered immune cells described herein or a pharmaceutical composition comprising the engineered immune cells. Also provided are methods for delaying progression of a mesothelin-associated cancer in a subject comprising administering to the subject the engineered immune cells described herein or a pharmaceutical composition comprising the engineered immune cells.

The disclosure also provides methods for inhibiting the proliferation or reducing a mesothelin-expressing cancer cell population, the methods comprising contacting a population of cells comprising a mesothelin-expressing cancer cell with an engineered immune cell provided herein (e.g., a T cell or an NK cell comprising a CAR and one or more exogenous polypeptides). In some embodiments, administration of an engineered immune cell provided herein reduces the quantity, number, amount, or percentage of mesothelin-expressing cancer cells in a subject by at least 25%, at least 30%, at least 40%, at least 50%, at least 65%, at least 75%, at least 85%, at least 95%, or at least 99% relative as compared to the quantity, number, amount, or percentage of mesothelin-expressing cancer cells in the subject prior to the administration. In some embodiments, the subject is a human subject.

In some embodiments, the engineered immune cells are administered to a subject having a mesothelin-associated cancer. In some embodiments, the mesothelin-associated cancer is mesothelioma (e.g., malignant mesothelioma, a pericardial mesothelioma, pleural mesothelioma, and or a peritoneal mesothelioma), ovarian cancer, pancreatic cancer (e.g., exocrine pancreatic cancer (e.g., adenocarcinomas, acinar cell carcinomas, adenosquamous carcinomas, colloid carcinomas, undifferentiated carcinomas with osteoclast-like giant cells, hepatoid carcinomas, intraductal papillary-mucinous neoplasms, mucinous cystic neoplasms, pancreatoblastomas, serous cystadenomas, signet ring cell carcinomas, solid and pseudopapillary tumors, pancreatic ductal carcinomas, and undifferentiated carcinomas) and endocrine pancreatic cancer (e.g., insulinomas and glucagonomas)), brain cancer (e.g., glioblastoma), lung cancer (e.g., lung adenocarcinoma), bladder cancer, breast cancer, cervical cancer, colorectal cancer, head and neck cancer, liver cancer, kidney cancer, lymphoma, leukemia (e.g., acute myeloid leukemia), skin cancer (e.g., melanoma), neuroblastoma, ovarian cancer, thyroid cancer (e.g., thymoma and thymic carcinoma), sarcoma (e.g., synovial sarcoma), gastric cancer, pleural cancer, glioblastoma, esophageal cancer, gastric cancer, urothelial cancer, ureter cancer, endometrial cancer, penile cancer, stomach cancer, squamous cell carcinoma, cholangiocarcinoma, and any combination thereof.

In some embodiments, the subject is administered a nonmyeloablative lymphodepleting chemotherapy prior to the administration of an engineered immune cell provided herein (or a composition including an engineered immune cell provided herein). The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered to the subject. In some embodiments, about 60 mg/kg of cyclophosphamide is administered for two days after which around 25 mg/m² fludarabine is administered for five days. In some embodiments, the nonmyeloablative lymphodepleting immunotherapy comprises administration of an anti-CD52 antibody (e.g., alemtuzumab) or an anti-CD20 antibody. Exemplary anti-CD20 antibodies include rituximab, ofatumumab, ocrelizumab, obinutuzumab, ibritumomab or iodine-131 tositumomab.

In certain embodiments, a growth factor that promotes the growth and activation of the engineered immune cells (e.g., NK cells) may be administered to the subject either concomitantly with the engineered immune cells or subsequent to the engineered immune cells. Examples of suitable growth factors include interleukin IL-2, IL-7, IL-15, IL-15Ra, and IL-12, which can be administered alone or in various combinations, such as fusion proteins including IL-15 and IL-15Ra, or the combinations of IL-2 and IL-7; IL-2 and IL-15; IL-7 and IL-15; IL-2, IL-7, and IL-15; IL-12 and IL-7; IL-12 and IL-15; or IL-12 and IL-2.

The engineered immune cells (or pharmaceutical compositions comprising the cells) can be administered by a number of routes, including parenteral administration, for example, intravenous, intraperitoneal, intramuscular, intrasternal, intratumoral or intraarticular injection, or infusion. In some embodiments, the engineered immune cells are targeted to the cancer cells in a subject via regional delivery directly to a tumor tissue in the subject. For example, in some embodiments, the engineered immune cells can be delivered intraperitoneally (IP) to the abdomen or peritoneal cavity of a subject. Such IP delivery can be performed via a port or pre-existing port (e.g., placed for delivery of chemotherapy agents). Other methods of regional delivery of engineered immune cells include catheter infusion into resection cavity, ultrasound guided intratumoral injection, hepatic artery infusion, or intrapleural delivery. In one embodiment, the engineered immune cells are targeted to the cancer via regional delivery directly to the tumor tissue. For example, in some embodiments, the engineered immune cells can be delivered intraperitoneally (IP) to the abdomen or peritoneal cavity. Such IP delivery can be performed via a port or pre-existing port placed for delivery of chemotherapy drugs. Other methods of regional delivery of engineered immune cells include catheter infusion into resection cavity, ultrasound guided intratumoral injection, hepatic artery infusion, or intrapleural delivery. In some embodiments, the engineered immune cells are delivered to a subject intratumorally. For example, in some embodiments, a catheter is placed at the tumor or metastasis site for administration of the engineered immune cells (or a composition including the engineered immune cells).

The engineered immune cells described herein may be used in a treatment regimen in combination with surgery, chemotherapy, radiation, immunosuppressive agents (e.g., cyclosporin, azathioprine, methotrexate, mycophenolate, and FK506), immunoablative agents (e.g., CAMPATH, anti-CD3 antibodies, cytotoxin, mucophenolic acid, fludarabine, rapamycin, FR901228) radiation therapy, gene therapy, DNA therapy, viral therapy, RNA therapy, immunotherapy, bone marrow transplantation, nanotherapy, monoclonal antibody therapy, or a combination of the foregoing. Any pharmaceutical agents used in combination with the engineered immune cells described herein can be administered before, during, or after administration of the engineered immune cells, depending on the desired effect. The engineered immune cells (or pharmaceutical compositions comprising the engineered immune cells) and the pharmaceutical agent may be administered to the subject by the same route or by different routes, and either at the same site or at a different site.

In some embodiments, the engineered immune cells provided herein (and compositions comprising the engineered immune cells) may be administered to the subject in combination with one or more other chemotherapeutic agents. Examples of chemotherapeutic agents include anthracyclines, vinca alkaloids, alkylating agents, therapeutic antibodies (e.g., alemtuzamab, gemtuzumab, rituximab, tositumomab), anti-metabolites (e.g., folic acid antagonists, pyrimidine analogs, purine analogs and adenosine deaminase inhibitors (e.g., fludarabine)), mTOR inhibitors, TNFR glucocorticoid induced TNFR related protein (GITR) agonists, proteasome inhibitors, and immunomodulators. Examples of anthracyclines that may be used include, e.g., doxorubicin, bleomycin, daunorubicin, daunorubicin liposomal, mitoxantrone, epirubicin, idarubicin, mitomycin C, geldanamycin, herbimycin, ravidomycin, and desacetylravidomycin. Examples of alkylating agents that may be used include, without limitation, nitrogen mustards, ethylenimine derivatives, alkyl sulfonates, nitrosoureas, triazenes, uracil mustard, chlormethine, cyclophosphamide, ifosfamide, melphalan, chlorambucil, pipobroman, triethylenemelamine, triethylenethiophosphoramine, temozolomide, thiotepa, busulfan, carmustine, lomustine, streptozocin, dacarbazine, oxaliplatin, dactinomycin, altretamine, carmustine, bendamustine, busulfan; carboplatin, lomustine, cisplatin, altretamine, ifosfamide, prednumustine, procarbazine, mechlorethamine, streptozocin, thiotepa, and bendamustine hydrochloride. Examples of vinca alkaloids that may be used include, e.g., vinorelbine tartrate, vincristine, vindesine, vinblastine, and vinorelbine. Examples of mTOR inhibitors that may be used include, e.g., temsirolimus, ridaforolimus, AP23573, everolimus, rapamycin, simapimod, emsirolimus, AZD8055, PF04691502, SF1126, and XL765. Examples of immunomodulators that may be used include, e.g., afutuzumab, pegfilgrastim, lenalidomide, thalidomide, actimid, and IRX-2. Examples of anthracyclines that may be used include, e.g., doxorubicin, bleomycin, daunorubicin, daunorubicin liposomal, mitoxantrone, epirubicin, idarubicin, mitomycin C, geldanamycin, herbimycin, ravidomycin, and desacetylravidomycin. Examples of proteosome inhibitors that may be used include bortezomib, carfilzomib, marizomib, ixazomib citrate, delanzomib, and ONX-0912. Additional chemotherapeutic agents that may be used include anastrozole, bicalutamide, bleomycin sulfate, busulfan, capecitabine, carboplatin, carmustine, chlorambucil, cisplatin, cladribine, cyclophosphamide, cytarabine, cytosine arabinoside, cytarabine liposome injection, dacarbazine, dactinomycin, daunorubicin hydrochloride, daunorubicin citrate liposome injection, dexamethasone, docetaxel, doxorubicin hydrochloride, etoposide, fludarabine phosphate, 5-fluorouracil, flutamide, tezacitibine, gemcitabine, hydroxyurea, idarubicin, ifosfamide, irinotecan, L-asparaginase, leucovorin calcium, melphalan, 6-mercaptopurine, methotrexate, mitoxantrone, mylotarg, paclitaxel, pentostatin, tamoxifen citrate, teniposide, 6-thioguanine, thiotepa, tirapazamine, and topotecan hydrochloride. Examples of immunotherapy includes that may be used include administration of immune checkpoint inhibitors. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). Examples of immune checkpoint inhibitors include antibodies and antigen binding fragments against each of the foregoing (e.g., anti-PD-1 antibodies and antigen-binding fragments thereof (e.g., nivolumab, pembrolizumab, and CT-011), and anti-CTLA-4 antibodies and antigen-binding fragments thereof (e.g., tremelimumab and ipilimumab).

In some embodiments, the engineered immune cells provided herein (and compositions comprising the engineered immune cells) may be administered to the subject in combination with a TNF-α converting enzyme (TACE) inhibitor. Without wishing to be bound by any particular theory, administration of a TACE inhibitor to the subject (e.g., before, during or after the engineered immune cells are administered to the subject) may reduce active shedding of mesothelin from the cell surface of mesothelin-expressing cells and improve the efficacy of the engineered immune cells. Examples of TACE inhibitors include ilomastat (also known as GM6001), batimastat, marimastat, KB-R7785 ((4-(N-hydroxyamino)-2R-isobutyl-3S-methylsuccinyl)-L-phenylglycine-N-methylamide), prinomastat, tanomastat, Ro 32-7315 ((E)-2(R)-[1(S)-(Hydroxycarbamoyl)-4-phenyl-3-butenyl]-2′-isobutyl-2′-(methanesulfonyl)-4-methylvalerohydrazide), cipemastat, CGS27023A (also known as MMI270B), TMI-1 (4-[[4-(2-butynyloxy)phenyl]sulfonyl]-N-hydroxy-2,2-dimethyl-(3S)thiomorpholinecarboxamide) and DPC-333 ([(2R)-2-{(3R)-3-amino-3-[4-(2-methylquinolin-4-ylmethoxy)phenyl]-2-oxopyrrolidin-1-yl}-N-hydroxy-4-methylpentanamide).

The engineered immune cells (or pharmaceutical compositions comprising the cells) may be administered to a subject before, during, after, or in various combinations relative to an additional therapy, such as immune checkpoint inhibitor therapy.

### Pharmaceutical Compositions

Also provided herein are pharmaceutical compositions comprising the engineered immune cells provided herein (e.g., engineered NK cells) and a pharmaceutically acceptable carrier. The pharmaceutical compositions can include a combination of one or more physiologically acceptable carriers. The pharmaceutical compositions may include buffers (e.g., neutral buffered saline, phosphate buffered saline and the like), antioxidants, carbohydrates, amino acids, antioxidants; chelating agents, and preservatives. The pH and exact concentration of the various components in a pharmaceutical composition are adjusted according to well-known parameters. In some embodiments, the pharmaceutical compositions are formulated for intravenous or intraperitoneal administration. In some embodiments, the pharmaceutical composition includes cryopreserved engineered immune cells.

### Articles of Manufacture or Kits

The disclosure also provides article of manufacture or kits including the engineered immune cells provided herein and/or the pharmaceutical compositions provided herein. The article of manufacture or kit can include a package insert comprising instructions for using the engineered immune cells and/or pharmaceutical compositions to treat or delay progression of cancer in a subject. Any of the engineered immune cells described herein may be included in the article of manufacture or kits. Suitable containers include, for example, bottles, vials, bags, and syringes. The container may be formed from a variety of materials such as glass, plastic (such as polyvinyl chloride or poly olefin), or metal alloy (such as stainless steel or hastelloy). In some embodiments, the container includes the pharmaceutical composition and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more additional therapeutic agents (e.g., a chemotherapeutic agent). Suitable containers for the one or more additional therapeutic agents include, for example, bottles, vials, bags, and syringes.

While the compositions and methods of this disclosure have been described in terms of preferred embodiments, it will be apparent to those of skill in the art that variations may be applied to the methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the disclosure as defined by the appended claims.

What is claimed is:

1. A chimeric antigen receptor comprising a sequence that is at least 85% identical to any one of SEQ ID NOs: 1-90.

2. The chimeric antigen receptor of claim 1, wherein the chimeric antigen receptor comprises a sequence that is at least 90% identical to any one of SEQ ID NOs: 1-90.

3. The chimeric antigen receptor of claim 2, wherein the chimeric antigen receptor comprises a sequence that is at least 95% identical to any one of SEQ ID NOs: 1-90.

4. The chimeric antigen receptor of claim 3, wherein the chimeric antigen receptor comprises a sequence of any one of SEQ ID NOs: 1-90.

5. A nucleic acid encoding a chimeric antigen receptor of any one of claims 1-4.

6. A vector comprising the nucleic acid of claim 5.

7. An engineered immune cell comprising a chimeric antigen receptor of any one of claims 1-5.

8. The engineered immune cell of claim 7, wherein the engineered immune cell is an engineered NK cell, T cell, or natural killer T (NKT) cell.

9. The engineered immune cell of claim 7 or 8, wherein the engineered immune cell further comprises one or more exogenous polypeptides.

10. The engineered immune cell of claim 9, wherein the one or more exogenous polypeptides is selected from the group consisting of interleukin-15 or a functional fragment thereof, interleukin-15 receptor alpha or a functional fragment thereof, or a transmembrane protein comprising IL-15 or a functional fragment thereof.

11. The engineered immune cell of claim 10, wherein the one or more exogenous polypeptides comprises the transmembrane protein.

12. The engineered immune cell of claim 10, wherein the one or more exogenous polypeptides comprises interleukin-15 or a functional fragment thereof.

13. The engineered immune cell of claim 10, wherein the one or more exogenous polypeptides comprises interleukin-15 receptor alpha or a functional fragment thereof.

14. The engineered immune cell of claim 9, wherein the engineered immune cell comprises a first exogenous polypeptide comprising interleukin-15 or a functional fragment thereof, and a second exogenous polypeptide comprising interleukin-15 receptor alpha or a functional fragment thereof.

15. The engineered immune cell of claim 11, wherein the transmembrane protein further comprises a sushi domain of IL-15 receptor alpha.

16. The engineered immune cell of any one of claims 9-15, wherein the one or more exogenous polypeptides comprises a transforming growth factor (TGF)-β dominant negative receptor.

17. The engineered immune cell of claim 16, wherein the TGF-β dominant negative receptor comprises the extracellular domain of TGF-β type II receptor.

18. The engineered immune cell of claim 16, wherein the TGF-β dominant negative receptor comprises the extracellular domain of TGF-β type I receptor.

19. The engineered immune cell of any one of claims 16-18, wherein the TGF-β receptor dominant negative further comprises the transmembrane domain of TGF-β type I receptor, TGF-β type II receptor, CD28, or CD8α.

20. A pharmaceutical composition comprising an engineered immune cell of any one of claims 7-19 and a pharmaceutically acceptable carrier.

21. A kit comprising the pharmaceutical composition of claim 20.

22. A method of treating a subject having a mesothelin-associated cancer, wherein the method comprises administering to the subject an engineered immune cell of any one of claims 7-19 or the pharmaceutical composition of claim 20.

23. The method of claim 22, wherein the administering is intravenous administration.

24. The method of claim 22 or 23, wherein the mesothelin-associated cancer is selected from the group consisting of: mesothelioma, ovarian cancer, pancreatic cancer, brain cancer, lung cancer, bladder cancer, breast cancer, cervical cancer, colorectal cancer, head and neck cancer, liver cancer, kidney cancer, lymphoma, leukemia, skin cancer, neuroblastoma, ovarian cancer, thyroid cancer, sarcoma, gastric cancer, pleural cancer, glioblastoma, esophageal cancer, gastric cancer, urothelial cancer, ureter cancer, endometrial cancer, penile cancer, stomach cancer, squamous cell carcinoma, cholangiocarcinoma, and any combination thereof.

25. The method of any one of claims 22-24, wherein the method further comprises administering an additional therapeutic agent to the subject.

26. The method of claim 25, wherein the additional therapeutic agent is a TNF-α converting enzyme (TACE) inhibitor.

27. The method of claim 26, wherein the TACE inhibitor is selected from ilomastat, batimastat, marimastat, KB-R7785, prinomastat, tanomastat, Ro 32-7315, cipemastat, CGS27023A, TMI-1, and DPC-333.

28. A method of making an engineered immune cell, the method comprising:
introducing into an immune cell a nucleic acid of claim 5 or a vector of claim 6.

29. The method of claim 26, wherein the immune cell is an NK cell, T cell, or natural killer T (NKT) cell.

30. The method of claim 28 or 29, wherein the method further comprises introducing into the immune cell nucleic acid encoding one or more exogenous polypeptides.

31. The method of claim 30, wherein the one or more exogenous polypeptides is selected from the group consisting of interleukin-15 or a functional fragment thereof, interleukin-15 receptor alpha or a functional fragment thereof, or a transmembrane protein comprising IL-15 or a functional fragment thereof.

32. The method of claim 31, wherein the one or more exogenous polypeptides comprises the transmembrane protein.

33. The method of claim 31, wherein the one or more exogenous polypeptides comprises interleukin-15 or a functional fragment thereof.

34. The method of claim 31 wherein the one or more exogenous polypeptides comprises interleukin-15 receptor alpha or a functional fragment thereof.

35. The method of claim 30, wherein the engineered immune cell comprises a first exogenous polypeptide comprising interleukin-15 or a functional fragment thereof, and a second exogenous polypeptide comprising interleukin-15 receptor alpha or a functional fragment thereof.

36. The method of claim 32, wherein the transmembrane protein further comprises a sushi domain of IL-15 receptor alpha.

37. The method of any one of claims 30-36, wherein the one or more exogenous polypeptides comprises a transforming growth factor (TGF)-β dominant negative receptor.

38. The method of claim 37, wherein the TGF-β dominant negative receptor comprises the extracellular domain of TGF-β type II receptor.

39. The method of claim 37, wherein the TGF-β dominant negative receptor comprises the extracellular domain of TGF-β type I receptor.

40. The method of any one of claims 37-39, wherein the TGF-β dominant negative receptor further comprises the transmembrane domain of TGF-β type I receptor, TGF-β type II receptor, CD28, or CD8α.
